(19) United States
(12) Patent Application Publication
(10) Pub. No.: US 2024/0228511 A1
(43) Pub. Date: Jul. 11, 2024

(54) SUBSTITUTED FUSED AZINES AS KRAS G12D INHIBITORS

(71) Applicant: Eli Lilly and Company, Indianapolis, IN (US)

(72) Inventors: David Anthony BARDA, Indianapolis, IN (US); Joshua Ryan CLAYTON, Fishers, IN (US); Jeffry Bernard FRANCISKOVICH, Zionsville, IN (US); Kelly Wayne FURNESS, Avon, IN (US); Douglas Linn GERNERT, Fishers, IN (US); James Robert HENRY, James City, FL (US); Richard Duane JOHNSTON, Greenfield, IN (US); Spencer Brian JONES, Indianapolis, IN (US); Jason Eric LAMAR, Indianapolis, IN (US); Adam Marc LEVINSON, Indianapolis, IN (US); Curren Tapfuma MBOFANA, Fishers, IN (US); Michael John RODRIQUEZ, Indianapolis, IN (US); Almudena RUBIO, Carmel, IN (US); Chong SI, Zionsville, IN (US); Gaiying ZHAO, Carmel, IN (US); Mohammed Sadegh ZIA-EBRAHIMI, Indianapolis, IN (US)

(21) Appl. No.: 18/288,314

(22) PCT Filed: Jun. 8, 2022
(86) PCT No.: PCT/US2022/032589

§ 371 Date: Oct. 25, 2023

Related U.S. Application Data
(60) Provisional application No. 63/208,662, filed on Jun. 9, 2021.

(51) Int. Cl. C07D 519/00; A61K 31/517; A61P 35/00; C07D 409/14; C07D 487/04

(52) U.S. Cl. CPC C07D 519/00; A61K 31/517; A61P 35/00; C07D 409/14; C07D 487/04

(57) ABSTRACT

The present invention provides compounds of the formula where R₁, R₂, R₃, R₄ₐ, R_{4b}, R_{4c}, R₅, R₆, X, Y, and Z are as described herein, pharmaceutically acceptable salts thereof, and methods of using these compounds and pharmaceutically acceptable salts thereof for treating patients for cancer.

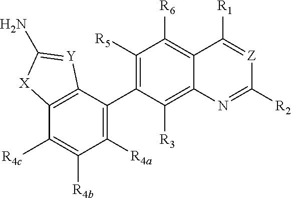

### BACKGROUND

The MAPK/ERK signaling pathway relays extracellular stimuli to the nucleus, thereby regulating diverse cellular responses including cell proliferation, differentiation, and apoptosis. KRas protein is an initiator of the MAPK/ERK signaling pathway and functions as a switch responsible for inducing cell division. In its inactive state, KRas binds guanosine diphosphate (GDP), effectively sending a negative signal to suppress cell division. In response to an extracellular signal, KRas is allosterically activated allowing for nucleotide exchange of GDP for guanosine triphosphate (GTP). In its GTP-bound active state, KRas recruits and activates proteins necessary for the propagation of growth factor induced signaling, as well as other cell signaling receptors. Examples of the proteins recruited by KRas-GTP are c-Raf and PI3-kinase. KRas, as a GTP-ase, converts the bound GTP back to GDP, thereby returning itself to an inactive state, and again propagating signals to suppress cell division. KRas gain of function mutations exhibit an increased degree of GTP binding and a decreased ability to convert GTP into GDP. The result is an increased MAPK/ERK signal which promotes cancerous cell growth. Missense mutations of KRas at codon 12 are the most common mutations and markedly diminish GTPase activity.

Oncogenic KRas mutations have been identified in approximately 30% of human cancers and have been demonstrated to activate multiple downstream signaling pathways. Despite the prevalence of KRas mutations, it has been a difficult therapeutic target. (Cox, A. D. Drugging the Undruggable RAS: Mission Possible? Nat. Rev. Drug Disc. 2014, 13, 828-851; Pylayeva-Gupta, y et al. RAS Oncogenes: Weaving a Tumorigenic Web. Nat. Rev. Cancer 2011, 11, 761-774).

Thus far, work has focused on KRas G12C mutant inhibitors (e.g., WO2019/099524, WO2020/081282, WO2020/101736, and WO2020/146613 disclose KRas G12C inhibitors), whereas WO2021/041671 discloses small molecules inhibitors of KRas G12D and WO2017/011920 discloses small molecule inhibitors of KRas G12C, G12D, and G12V.

There remains a need to provide alternative, small molecule KRas inhibitors. In particular, there is a need to provide more potent, orally deliverable KRas inhibitors that are useful for treating cancer. More particularly, there is a need to provide small molecule inhibitors that specifically inhibit KRas GTP activity. There is also a need to provide small molecule KRas inhibitors that exhibit greater efficacy at the same or reduced KRas inhibitory activity. Further, there is a desire to provide KRas inhibitors that exhibit better pharmacokinetic/pharmacodynamic properties. Also, there is a need to provide more potent KRas inhibitors that exhibit increased efficacy with reduced or minimized untoward or undesired effects. The present invention addresses one or more of these needs by providing novel KRas inhibitors.

### SUMMARY

Compounds of Formula I:

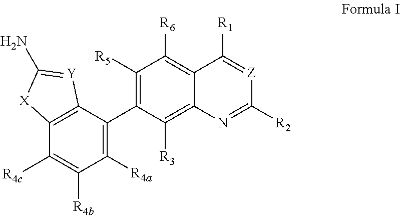

pharmaceutically acceptable salts thereof, and pharmaceutical compositions thereof, are provided herein. In Formula I,
X is —O— or —S—;
Y is —C(CN)— or —N—;
Z is —C(H)— or —N—;
R₁ is H, azetidine, pyrrolidine, piperidine, or N-linked piperazine, wherein the azetidine, pyrrolidine, piperidine, or N-linked piperazine are optionally substituted with C₁₋₄ alkyl or C₁₋₄ heteroalkyl, wherein the C₁₋₄ alkyl, C₁₋₄ heteroalkyl are optionally substituted by halogen or oxo, wherein the azetidine, pyrrolidine, piperidine, or N-linked piperazine are optionally bridged by the C₁₋₄ alkyl or C₁₋₄ heteroalkyl, and wherein the azetidine, pyrrolidine, piperidine, or N-linked piperazine are optionally fused with the C₁₋₄ alkyl or C₁₋₄ heteroalkyl to form a bicyclic ring;
R₂ is H, —O—CH₂—R₇, or —O—CH(CH₃)—R₇, wherein R₇ is azetidine, pyrrolidine, or tetrahydrofuran, wherein the azetidine, pyrrolidine, or tetrahydrofuran are optionally substituted with one or more halogen, hydroxyl, C₁₋₄ alkyl, or C₁₋₄ alkenyl, wherein the C₁₋₄ alkyl is optionally substituted with one or more halogen or hydroxyl, wherein the azetidine, pyrrolidine, or tetrahydrofuran are optionally fused with the C₁₋₄ alkyl to form a bicyclic ring, and wherein if R₂ is H then R₁ is not H;
R₃ and R₅ are each independently H, halogen, —C₀₋₃ alkyl-cyclopropyl, —C₁₋₆ alkyl optionally substituted 1-3 times with R₈, or —O—C₁₋₆ alkyl optionally substituted 1-3 times with R₈;
R₄ₐ, R_{4b}, and R_{4c}, are each independently H, halogen, or —C₁₋₆ alkyl optionally substituted 1-3 times with R₈;
R₆ is H, —CH₂OH, —CH₂—O—CH₃; and
R₈ is independently at each occurrence halogen, oxo, hydroxy, —C₁₋₄ alkyl, or —O—C₁₋₄ alkyl.

Methods of using the compounds of Formula I, pharmaceutically acceptable salts thereof, and pharmaceutical compositions thereof, to treat cancer, in particular for the treatment of lung cancer, pancreatic cancer, cervical cancer, esophageal cancer, endometrial cancer, ovarian cancer, cholangiocarcinoma, and colorectal cancer. The methods include administering a therapeutically effective amount of a compound of Formula I, or a pharmaceutically acceptable salt thereof, to a patient in need.

Also provided herein, are compounds of Formula I, and pharmaceutically acceptable salts thereof, for use in therapy. Further provided herein, are the compounds of Formula I, and pharmaceutically acceptable salts thereof, for use in the treatment of cancer, in particular for the treatment of lung cancer, pancreatic cancer, cervical cancer, esophageal cancer, endometrial cancer, ovarian cancer, cholangiocarcinoma, and colorectal cancer. The use of compounds of Formula I, or pharmaceutically acceptable salts thereof, in the manufacture of a medicament for treating cancer, in particular for the treatment of lung cancer, pancreatic cancer, cervical cancer, esophageal cancer, endometrial cancer, ovarian cancer, cholangiocarcinoma, and colorectal cancer.

### DETAILED DESCRIPTION

Novel inhibitors of the KRas gain of function mutation G12D are described herein. These new compounds could address the needs noted above for inhibitors of KRas GTP activity in gain of function mutants in the treatment of cancers such as lung cancer, colorectal cancer, pancreatic cancer, bladder cancer, cervical cancer, endometrial cancer, ovarian cancer, cholangiocarcinoma or esophageal cancer. Some of these new KRas G12D mutant inhibitor compounds are selective to KRas G12D mutants over wild-type KRas (and likely other mutant types such as G12C or G12V). Additionally, some of these new KRas G12D mutant inhibitor compounds are non-selective and inhibit both wild-type KRas and KRas G12D mutants (and possibly other mutant types such as G12C or G12V).

The present invention provides a compound of Formula I or a pharmaceutically acceptable salt thereof.

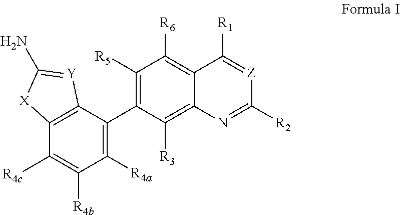

### In Formula I,

X can be —O— or —S—;
Y can be —C(CN)— or —N—;
Z can be —C(H)— or —N—;
R₁ can be H, azetidine, pyrrolidine, piperidine, or N-linked piperazine, wherein the azetidine, pyrrolidine, piperidine, or N-linked piperazine are optionally substituted with C₁₋₄ alkyl or C₁₋₄ heteroalkyl, wherein the C₁₋₄ alkyl, C₁₋₄ heteroalkyl are optionally substituted by halogen or oxo, wherein the azetidine, pyrrolidine, piperidine, or N-linked piperazine are optionally bridged by the C₁₋₄ alkyl or C₁₋₄ heteroalkyl, and wherein the azetidine, pyrrolidine, piperidine, or N-linked piperazine are optionally fused with the C₁₋₄ alkyl or C₁₋₄ heteroalkyl to form a bicyclic ring;
R₂ can be H, —O—CH₂—R₇, or —O—CH(CH₃)—R₇, wherein R₇ is azetidine, pyrrolidine, or tetrahydrofuran, wherein the azetidine, pyrrolidine, or tetrahydrofuran are optionally substituted with one or more halogen, hydroxyl, C₁₋₄ alkyl, or C₁₋₄ alkenyl, wherein the C₁₋₄ alkyl is optionally substituted with one or more halogen or hydroxyl, wherein the azetidine, pyrrolidine, or tetrahydrofuran are optionally fused with the C₁₋₄ alkyl to form a bicyclic ring, and wherein if R₂ is H then R₁ is not H;
R₃ and R₅ can each independently be H, halogen, —C₀₋₃ alkyl-cyclopropyl, —C₁₋₆ alkyl optionally substituted 1-3 times with R₈, or —O—C₁₋₆ alkyl optionally substituted 1-3 times with R₈;
R₄ₐ, R_{4b}, and R_{4c}, can each independently be H, halogen, or —C₁₋₆ alkyl optionally substituted 1-3 times with R₈;
R₆ can be H, —CH₂OH, —CH₂—O—CH₃; and
R₈ can independently at each occurrence be halogen, oxo, hydroxy, —C₁₋₄ alkyl, or —O—C₁₋₄ alkyl.

In an embodiment the present invention provides a compound of Formula II:

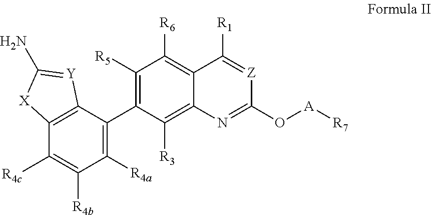

where R₁, R₃, R₄, R₅, R₇, X, Y, and Z are as defined above and A is —CH₂— or —CH(CH₃)—, or a pharmaceutically acceptable salt thereof.

In another embodiment the present invention provides a compound of Formula III:

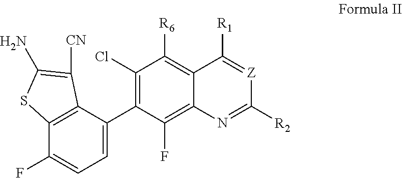

where R₁, R₂, R₈, and Z are as defined above, or a pharmaceutically acceptable salt thereof.

In a further embodiment the present invention provides a compound of Formula IV:

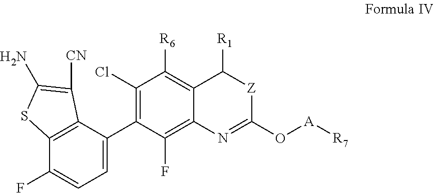

where R₁, R₆, R₇, and Z are as defined above and A is —CH₂— or —CH(CH₃)—, or a pharmaceutically acceptable salt thereof.

As used herein, the term halogen means fluoro (F), chloro (Cl), bromo (Br), or iodo (I). As used herein, the term alkyl means saturated linear or branched-chain monovalent hydrocarbon radicals of one to six carbon atoms, e.g., “—C₁₋₆ alkyl” or “—C₁₋₄ alkyl”. Examples of alkyls include, but are not limited to, methyl, ethyl, propyl, 1-propyl, isopropyl, butyl, pentyl, and hexyl. As used herein, the term “oxo” means an oxygen double-bonded to a carbon, i.e., a ketone. As used herein, the term heteroalkyl means saturated linear or branched-chain monovalent hydrocarbon radicals containing two to four carbon atoms and at least one heteroatom, e.g., “—C₂₋₄ heteroalkyl.” Examples of heteroatoms include, but are not limited to nitrogen and oxygen. In cases where a zero is indicated, e.g., —C₀₋₃ alkyl-cyclopropyl, the alkyl component of the substituent group can be absent, thus, if R₃ or R₅ of Formula I is a cyclopropyl group with no lead alkyl, the substituent would be described by the —C₀₋₃ alkyl-cyclopropyl substituent as described for R₃ or R₅ (i.e., the substituent group would be —C₀-cyclopropyl).

For R₁, the azetidine, pyrrolidine, piperidine, or N-linked piperazine are optionally bridged by the C₁₋₄ alkyl or C₂₋₄ heteroalkyl. As used herein, the term “bridged” for the R₁ group means the R₁ group is bicyclic with the C₁₋₄ alkyl or C₂₋₄ heteroalkyl connecting to two, non-adjacent atoms of the azetidine, pyrrolidine, piperidine, or N-linked piperazine ring. Examples of bridged N-linked piperazine ring groups include:

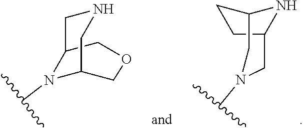 [SMILES: *N1C2CNCC1COC2 ; *N1CC2CCC(C1)N2]

As used herein, the term “fused” for the R₁ group means the R₁ group is bicyclic with the C₁₋₄ alkyl or C₂₋₄ heteroalkyl connecting to two, adjacent atoms of the azetidine, pyrrolidine, piperidine, or N-linked piperazine ring. Examples of fused R₁ groups include:

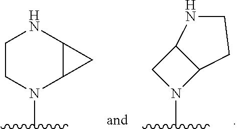 [SMILES: *N1CCNC2CC21 ; *N1CC2NCCC21]

In R₁, the azetidine, pyrrolidine, and piperidine groups are not specified to be bonded through a carbon or nitrogen and could be either. Similarly, C₁₋₄ alkyl or C₁₋₄ heteroalkyl substitutions onto the R₁ azetidine, pyrrolidine, and piperidine groups can be on a carbon or heteroatom.

For R₇, the azetidine, pyrrolidine, or tetrahydrofuran are optionally fused with a C₁₋₄ alkyl to form a bicyclic ring. As used herein, the term “fused” for the R₇ group means the R₇ group is bicyclic with the C₁₋₄ alkyl connecting to two, adjacent atoms of the azetidine, pyrrolidine, or tetrahydrofuran ring. Examples of fused R₇ groups include:

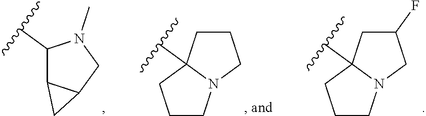

In R₇, the azetidine, pyrrolidine, or tetrahydrofuran groups are not specified to be bonded through a carbon or nitrogen and could be either. Similarly, C₁₋₄ alkyl or C₁₋₄ alkenyl substitutions onto the R₇ azetidine, pyrrolidine, or tetrahydrofuran groups can be on a carbon or heteroatom.

In an embodiment of a compound of Formulae I or II or a pharmaceutically acceptable salt thereof, X is —S—.

In another embodiment of a compound of Formulae I or II or a pharmaceutically acceptable salt thereof, Y is —C(CN)—.

In a further embodiment of a compound of any of Formulae I, II, III, or IV or a pharmaceutically acceptable salt thereof, Z is —N—.

In an additional embodiment of a compound of any of Formulae I, II, III, or IV or a pharmaceutically acceptable salt thereof, R₁ is H.

In another embodiment of a compound of any of Formulae I, II, III, or IV or a pharmaceutically acceptable salt thereof, R₁ is azetidine, pyrrolidine, piperidine, or N-linked piperazine.

In a further embodiment of a compound of any of Formulae I, II, III, or IV or a pharmaceutically acceptable salt thereof, R₁ is N-linked piperazine.

In an additional embodiment of a compound of any of Formulae I, II, III, or IV or a pharmaceutically acceptable salt thereof, R₁ is

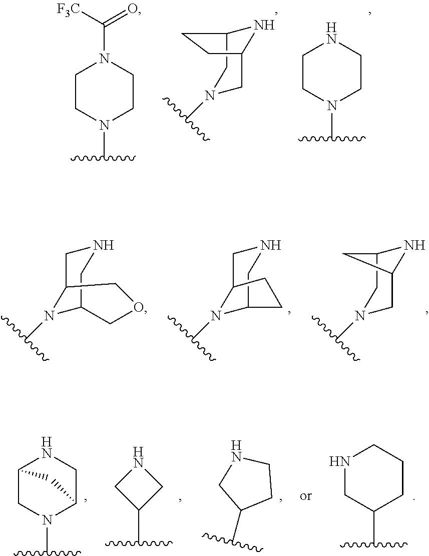

In another embodiment of a compound of any of Formulae I, II, III, or IV or a pharmaceutically acceptable salt thereof, R₁ is

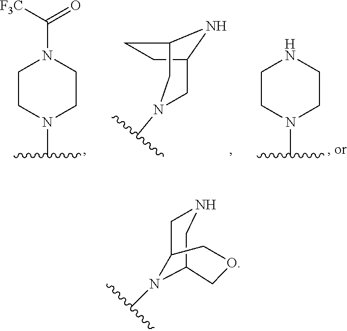 [SMILES: *N1CCN(C(=O)C(F)(F)F)CC1 ; *N1CC2CCC(C1)N2 ; *N1CCNCC1 ; *N1C2CNCC1COC2]

In a further embodiment of a compound of any of Formulae I, II, III, or IV or a pharmaceutically acceptable salt thereof, R₁ is

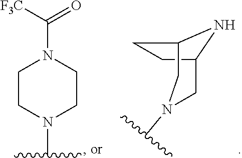 [SMILES: *N1CCN(C(=O)C(F)(F)F)CC1 ; *N1CC2CCC(C1)N2]

In an additional embodiment of a compound of Formulae I or III or a pharmaceutically acceptable salt thereof, R₂ is —O—CH₂—R₇.

In another embodiment of a compound of Formulae II or IV or a pharmaceutically acceptable salt thereof, R₇ is pyrrolidine.

In a further embodiment of a compound of Formulae I or III or a pharmaceutically acceptable salt thereof, R₂ is

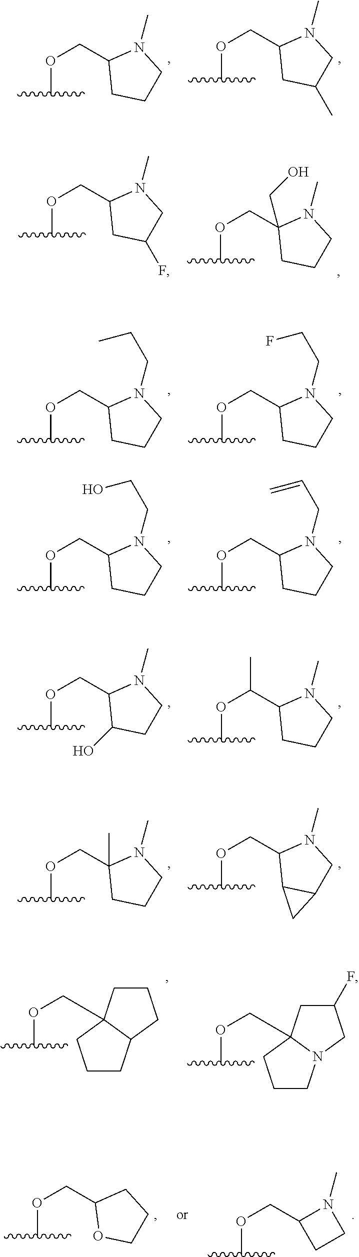

In an additional embodiment of a compound of Formulae I or III or a pharmaceutically acceptable salt thereof, R₂ is

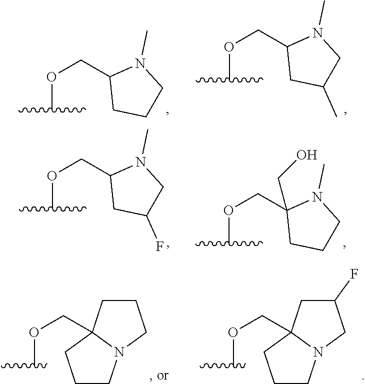

In another embodiment of a compound of Formulae I or II or a pharmaceutically acceptable salt thereof, R₃ and R₅ are each independently halogen, —C₀₋₃ alkyl-cyclopropyl, —C₁₋₆ alkyl optionally substituted 1-3 times with R₈, or —O—C₁₋₆ alkyl optionally substituted 1-3 times with R₈.

In a further embodiment of a compound of Formulae I or II or a pharmaceutically acceptable salt thereof, R₃ is F.

In an additional embodiment of a compound of Formulae I or III or a pharmaceutically acceptable salt thereof, R_{4c} is F or —CH₃.

In another embodiment of a compound of Formulae I or II or a pharmaceutically acceptable salt thereof, R₅ is Cl.

In a further embodiment of a compound of Formulae I or II or a pharmaceutically acceptable salt thereof, X is S, Y is —C(CN)—, R₃ is F, R₄ₐ is H, R_{4b} is H, R_{4c} is F, and R₅ is Cl.

In an additional embodiment of a compound of Formulae III or IV or a pharmaceutically acceptable salt thereof, R₁ is

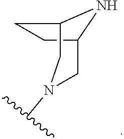 [SMILES: *N1CC2CCC(C1)N2]

In another embodiment of a compound of Formulae III or IV or a pharmaceutically acceptable salt thereof, R₁ is

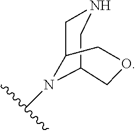 [SMILES: *N1C2CNCC1COC2]

In a further embodiment of a compound of Formulae III or IV or a pharmaceutically acceptable salt thereof, R₁ is

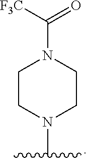 [SMILES: *N1CCN(C(=O)C(F)(F)F)CC1]

In an additional embodiment of a compound of Formulae III or IV or a pharmaceutically acceptable salt thereof, R₁ is

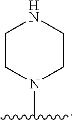 [SMILES: *N1CCNCC1]

In another embodiment of a compound of Formulae II or IV or a pharmaceutically acceptable salt thereof, A is —CH₂—.

In a further embodiment of a compound of any of Formulae I, II, III, or IV or a pharmaceutically acceptable salt thereof, R₆ is H.

Examples of compounds described herein include the compounds of Table 1 and pharmaceutically acceptable salts thereof.

TABLE 1
Example Compounds

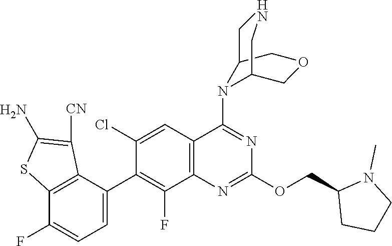 [SMILES: CN1CCC[C@H]1COc1nc(N2C3CNCC2COC3)c2cc(Cl)c(-c3ccc(F)c4sc(N)c(C#N)c34)c(F)c2n1]

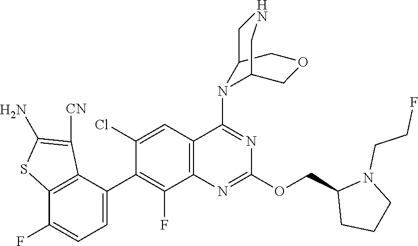 [SMILES: N#Cc1c(N)sc2c(F)ccc(-c3c(Cl)cc4c(N5C6CNCC5COC6)nc(OC[C@@H]5CCCN5CCF)nc4c3F)c12]

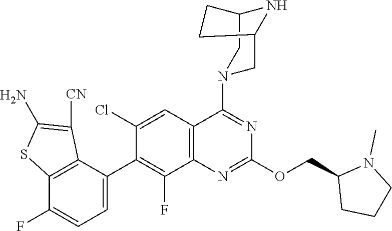 [SMILES: CN1CCC[C@H]1COc1nc(N2CC3CCC(C2)N3)c2cc(Cl)c(-c3ccc(F)c4sc(N)c(C#N)c34)c(F)c2n1]

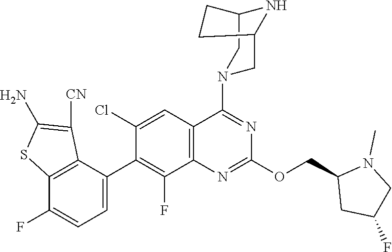 [SMILES: CN1C[C@H](F)C[C@H]1COc1nc(N2CC3CCC(C2)N3)c2cc(Cl)c(-c3ccc(F)c4sc(N)c(C#N)c34)c(F)c2n1]

(isomer 2)

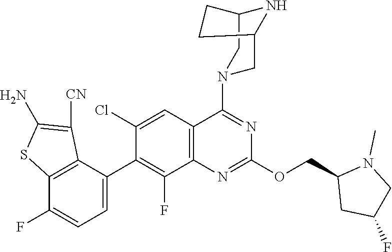 [SMILES: CN1C[C@H](F)C[C@H]1COc1nc(N2CC3CCC(C2)N3)c2cc(Cl)c(-c3ccc(F)c4sc(N)c(C#N)c34)c(F)c2n1]

(isomer 1)

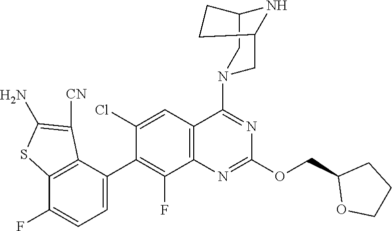 [SMILES: N#Cc1c(N)sc2c(F)ccc(-c3c(Cl)cc4c(N5CC6CCC(C5)N6)nc(OC[C@H]5CCCO5)nc4c3F)c12]

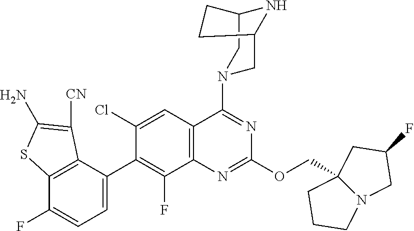 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

(isomer 2)

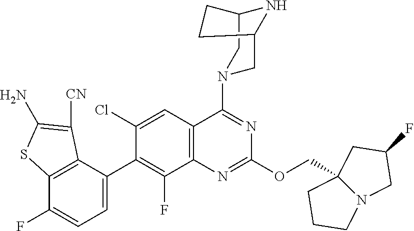 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

(isomer 1)

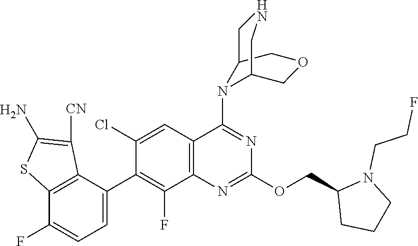 [SMILES: N#Cc1c(N)sc2c(F)ccc(-c3c(Cl)cc4c(N5C6CNCC5COC6)nc(OC[C@@H]5CCCN5CCF)nc4c3F)c12]

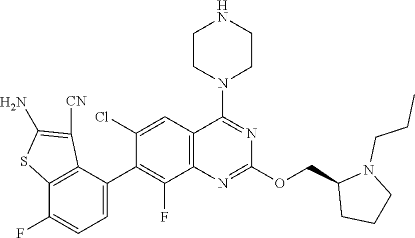 [SMILES: CCCN1CCC[C@H]1COc1nc(N2CCNCC2)c2cc(Cl)c(-c3ccc(F)c4sc(N)c(C#N)c34)c(F)c2n1]

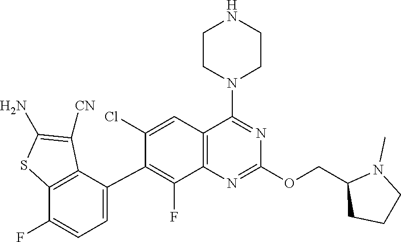 [SMILES: CN1CCC[C@H]1COc1nc(N2CCNCC2)c2cc(Cl)c(-c3ccc(F)c4sc(N)c(C#N)c34)c(F)c2n1]

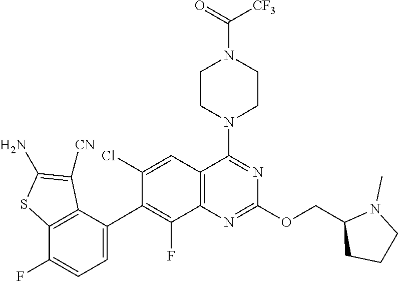 [SMILES: CN1CCC[C@H]1COc1nc(N2CCN(C(=O)C(F)(F)F)CC2)c2cc(Cl)c(-c3ccc(F)c4sc(N)c(C#N)c34)c(F)c2n1]

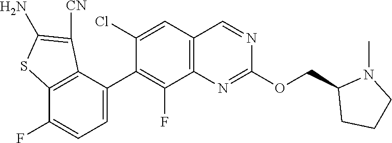 [SMILES: CN1CCC[C@H]1COc1ncc2cc(Cl)c(-c3ccc(F)c4sc(N)c(C#N)c34)c(F)c2n1]

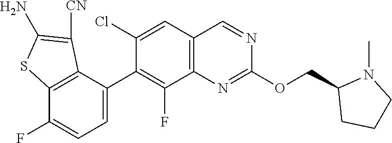 [SMILES: CN1CCC[C@H]1COc1ncc2cc(Cl)c(-c3ccc(F)c4sc(N)c(C#N)c34)c(F)c2n1]

(isomer 1)

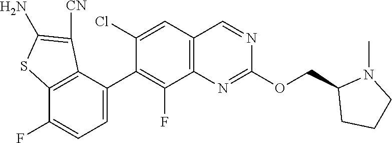 [SMILES: CN1CCC[C@H]1COc1ncc2cc(Cl)c(-c3ccc(F)c4sc(N)c(C#N)c34)c(F)c2n1]

(isomer 2)

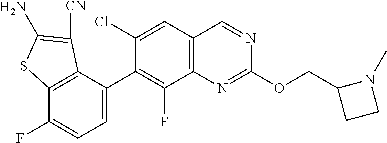 [SMILES: CN1CCC1COc1ncc2cc(Cl)c(-c3ccc(F)c4sc(N)c(C#N)c34)c(F)c2n1]

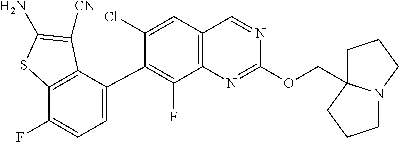 [SMILES: N#Cc1c(N)sc2c(F)ccc(-c3c(Cl)cc4cnc(OCC56CCCN5CCC6)nc4c3F)c12]

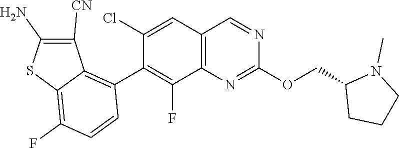 [SMILES: CN1CCC[C@@H]1COc1ncc2cc(Cl)c(-c3ccc(F)c4sc(N)c(C#N)c34)c(F)c2n1]

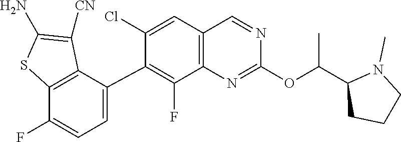 [SMILES: CC(Oc1ncc2cc(Cl)c(-c3ccc(F)c4sc(N)c(C#N)c34)c(F)c2n1)[C@@H]1CCCN1C]

(isomer 1)

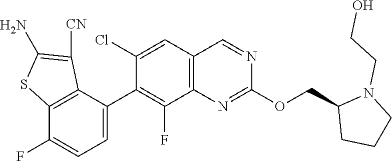 [SMILES: N#Cc1c(N)sc2c(F)ccc(-c3c(Cl)cc4cnc(OC[C@@H]5CCCN5CCO)nc4c3F)c12]

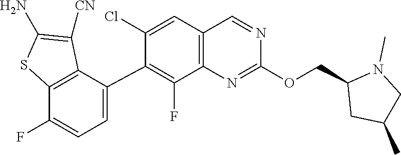 [SMILES: C[C@H]1C[C@@H](COc2ncc3cc(Cl)c(-c4ccc(F)c5sc(N)c(C#N)c45)c(F)c3n2)N(C)C1]

(isomer 1)

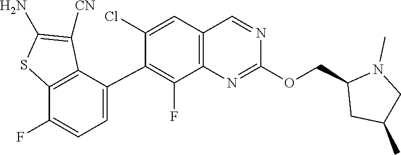 [SMILES: C[C@H]1C[C@@H](COc2ncc3cc(Cl)c(-c4ccc(F)c5sc(N)c(C#N)c45)c(F)c3n2)N(C)C1]

(isomer 2)

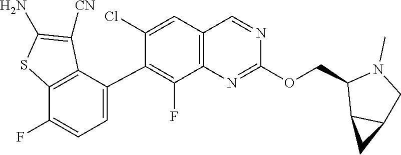 [SMILES: CN1C[C@H]2C[C@H]2[C@H]1COc1ncc2cc(Cl)c(-c3ccc(F)c4sc(N)c(C#N)c34)c(F)c2n1]

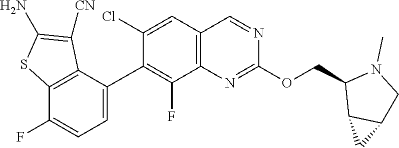 [SMILES: CN1C[C@@H]2C[C@@H]2[C@H]1COc1ncc2cc(Cl)c(-c3ccc(F)c4sc(N)c(C#N)c34)c(F)c2n1]

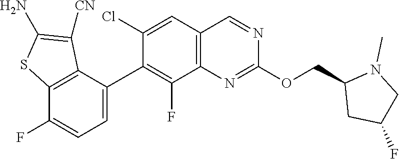 [SMILES: CN1C[C@H](F)C[C@H]1COc1ncc2cc(Cl)c(-c3ccc(F)c4sc(N)c(C#N)c34)c(F)c2n1]

(isomer 1)

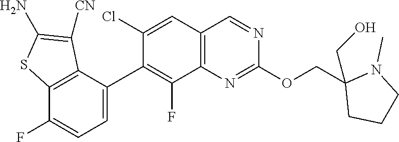 [SMILES: CN1CCCC1(CO)COc1ncc2cc(Cl)c(-c3ccc(F)c4sc(N)c(C#N)c34)c(F)c2n1]

(isomer 1)

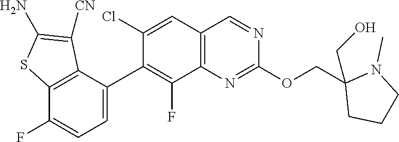 [SMILES: CN1CCCC1(CO)COc1ncc2cc(Cl)c(-c3ccc(F)c4sc(N)c(C#N)c34)c(F)c2n1]

(isomer 2)

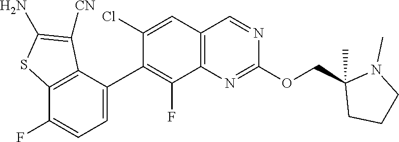 [SMILES: CN1CCC[C@@]1(C)COc1ncc2cc(Cl)c(-c3ccc(F)c4sc(N)c(C#N)c34)c(F)c2n1]

(isomer 2)

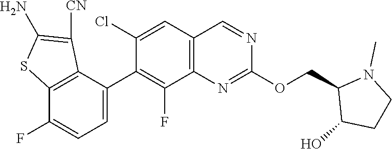 [SMILES: CN1CC[C@H](O)[C@H]1COc1ncc2cc(Cl)c(-c3ccc(F)c4sc(N)c(C#N)c34)c(F)c2n1]

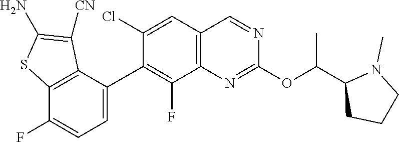 [SMILES: CC(Oc1ncc2cc(Cl)c(-c3ccc(F)c4sc(N)c(C#N)c34)c(F)c2n1)[C@@H]1CCCN1C]

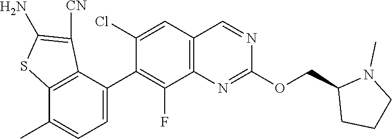 [SMILES: Cc1ccc(-c2c(Cl)cc3cnc(OC[C@@H]4CCCN4C)nc3c2F)c2c(C#N)c(N)sc12]

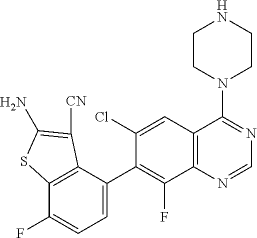 [SMILES: N#Cc1c(N)sc2c(F)ccc(-c3c(Cl)cc4c(N5CCNCC5)ncnc4c3F)c12]

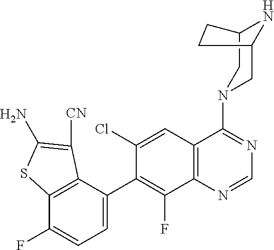 [SMILES: N#Cc1c(N)sc2c(F)ccc(-c3c(Cl)cc4c(N5CC6CCC(C5)N6)ncnc4c3F)c12]

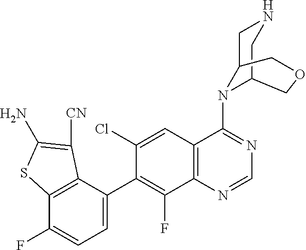 [SMILES: N#Cc1c(N)sc2c(F)ccc(-c3c(Cl)cc4c(N5C6CNCC5COC6)ncnc4c3F)c12]

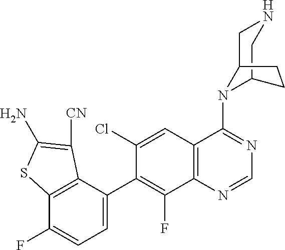 [SMILES: N#Cc1c(N)sc2c(F)ccc(-c3c(Cl)cc4c(N5C6CCC5CNC6)ncnc4c3F)c12]

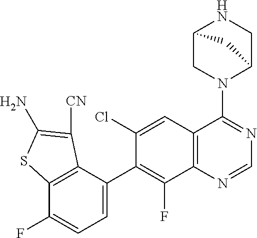 [SMILES: N#Cc1c(N)sc2c(F)ccc(-c3c(Cl)cc4c(N5C[C@@H]6C[C@H]5CN6)ncnc4c3F)c12]

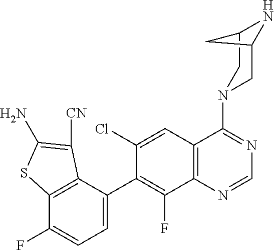 [SMILES: N#Cc1c(N)sc2c(F)ccc(-c3c(Cl)cc4c(N5CC6CC(C5)N6)ncnc4c3F)c12]

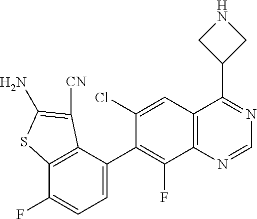 [SMILES: N#Cc1c(N)sc2c(F)ccc(-c3c(Cl)cc4c(C5CNC5)ncnc4c3F)c12]

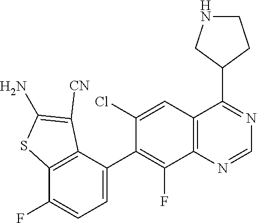 [SMILES: N#Cc1c(N)sc2c(F)ccc(-c3c(Cl)cc4c(C5CCNC5)ncnc4c3F)c12]

(isomer 1)

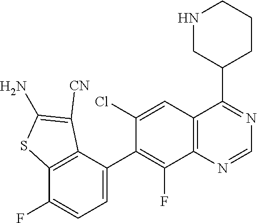 [SMILES: N#Cc1c(N)sc2c(F)ccc(-c3c(Cl)cc4c(C5CCCNC5)ncnc4c3F)c12]

(diastereomer 1)

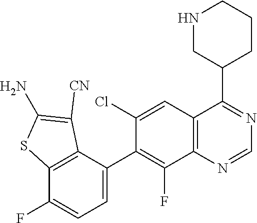 [SMILES: N#Cc1c(N)sc2c(F)ccc(-c3c(Cl)cc4c(C5CCCNC5)ncnc4c3F)c12]

(diastereomer 2)

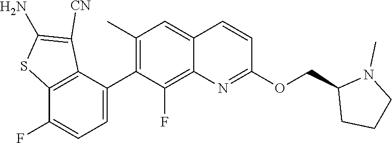 [SMILES: Cc1cc2ccc(OC[C@@H]3CCCN3C)nc2c(F)c1-c1ccc(F)c2sc(N)c(C#N)c12]

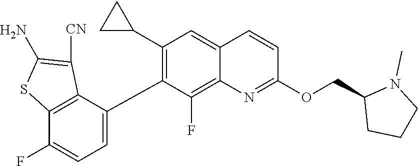 [SMILES: CN1CCC[C@H]1COc1ccc2cc(C3CC3)c(-c3ccc(F)c4sc(N)c(C#N)c34)c(F)c2n1]

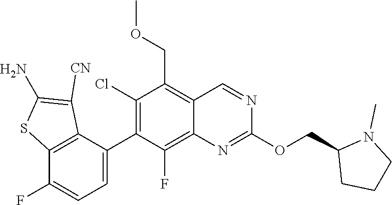 [SMILES: COCc1c(Cl)c(-c2ccc(F)c3sc(N)c(C#N)c23)c(F)c2nc(OC[C@@H]3CCCN3C)ncc12]

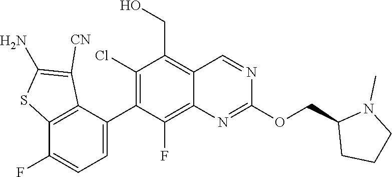 [SMILES: CN1CCC[C@H]1COc1ncc2c(CO)c(Cl)c(-c3ccc(F)c4sc(N)c(C#N)c34)c(F)c2n1]

Preferred examples of compounds described herein include the compounds of Table 2 and pharmaceutically acceptable salts thereof.

TABLE 2
Preferred Example Compounds

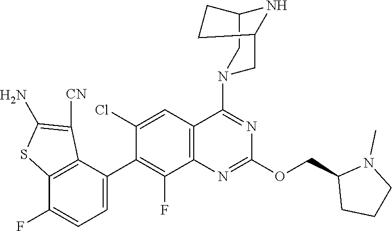 [SMILES: CN1CCC[C@H]1COc1nc(N2CC3CCC(C2)N3)c2cc(Cl)c(-c3ccc(F)c4sc(N)c(C#N)c34)c(F)c2n1]

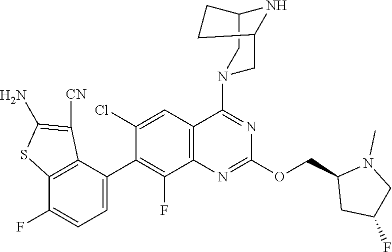 [SMILES: CN1C[C@H](F)C[C@H]1COc1nc(N2CC3CCC(C2)N3)c2cc(Cl)c(-c3ccc(F)c4sc(N)c(C#N)c34)c(F)c2n1]

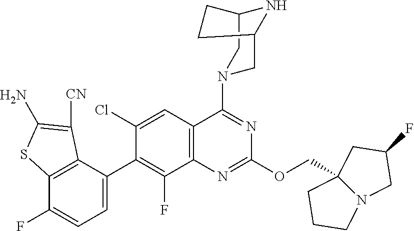 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

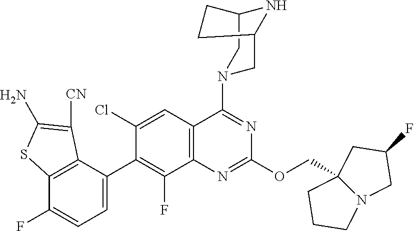 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

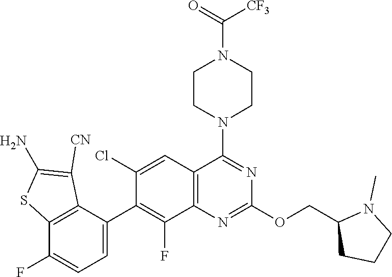 [SMILES: CN1CCC[C@H]1COc1nc(N2CCN(C(=O)C(F)(F)F)CC2)c2cc(Cl)c(-c3ccc(F)c4sc(N)c(C#N)c34)c(F)c2n1]

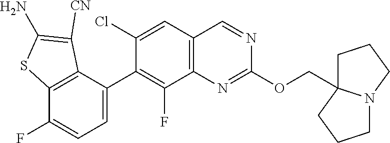 [SMILES: N#Cc1c(N)sc2c(F)ccc(-c3c(Cl)cc4cnc(OCC56CCCN5CCC6)nc4c3F)c12]

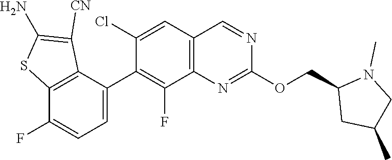 [SMILES: C[C@H]1C[C@@H](COc2ncc3cc(Cl)c(-c4ccc(F)c5sc(N)c(C#N)c45)c(F)c3n2)N(C)C1]

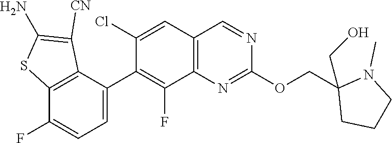 [SMILES: CN1CCCC1(CO)COc1ncc2cc(Cl)c(-c3ccc(F)c4sc(N)c(C#N)c34)c(F)c2n1]

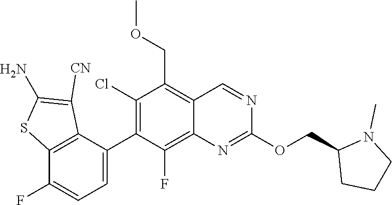 [SMILES: COCc1c(Cl)c(-c2ccc(F)c3sc(N)c(C#N)c23)c(F)c2nc(OC[C@@H]3CCCN3C)ncc12]

Also provided herein are pharmaceutical compositions comprising a compound according to any one of Formulae I-IV, or a pharmaceutically acceptable salt thereof, and a pharmaceutically acceptable carrier, diluent, or excipient.

Further provided herein are methods of treating cancer, comprising administering to a patient in need thereof, an effective amount of a compound according to any one of Formulae I-IV, or a pharmaceutically acceptable salt thereof. The cancer can be lung cancer, colorectal cancer, pancreatic cancer, bladder cancer, cervical cancer, endometrial cancer, ovarian cancer, cholangiocarcinoma, or esophageal cancer. The cancer can more specifically be non-small cell lung cancer, pancreatic cancer, or colorectal cancer. Still further specifically, the cancer can be non-small cell lung cancer.

Also provided herein is a method of treating cancer, comprising administering to a patient in need thereof, an effective amount of a compound according to any one of Formulae I-IV, or a pharmaceutically acceptable salt thereof, in which the cancer has one or more cells that express a mutant KRas G12D protein. In this method, the cancer can be non-small cell lung carcinoma, in which the cancer has one or more cells that express a KRas G12D mutant protein. Also in this method, the cancer is colorectal carcinoma in which the cancer has one or more cells that express a KRas G12D mutant protein. Further in this method, the cancer is mutant pancreatic cancer in which the cancer has one or more cells that express a KRas G12D mutant protein. Additionally in this method, the present invention comprising a method of treating KRas G12D mutant bearing cancers of other origins.

Further provided herein is a method of treating a patient with a cancer that has a KRas G12D mutation comprising administering to a patient in need thereof an effective amount of a compound according to any one of Formulae I-IV or a pharmaceutically acceptable salt thereof.

Additionally provided herein is a method of modulating a mutant KRas G12D enzyme in a patient in need thereof, by administering a compound according to any one of Formulae I-IV, or a pharmaceutically acceptable salt thereof. Preferably this method comprises inhibiting a human mutant KRas G12D enzyme.

Also provided herein is a method of treating cancer in a patient in need thereof, wherein the patient has a cancer that was determined to express the KRas G12D mutant protein. The method comprises administering to a patient an effective amount of a compound according to any one of Formulae I-IV, or a pharmaceutically acceptable salt thereof. The G12D mutational status of one or more cancer cells can be determined by a number of assays known in the art. Typically, one or more biopsies containing one or more cancer cells are obtained, and subjected to sequencing and/or polymerase chain reaction (PCR). Circulating cell-free DNA can also be used, e.g. in advanced cancers. Non-limiting examples of sequencing and PCR techniques used to determine the mutational status (e.g., G12D mutational status, in one or more cancer cells or in circulating cell-free DNA) include direct sequencing, next-generation sequencing, reverse transcription polymerase chain reaction (RT-PCR), multiplex PCR, and pyrosequencing and multi-analyte profiling.

Further provided herein is a compound or a pharmaceutically acceptable salt thereof according to any one of Formulae I-IV for use in therapy. The compound or a pharmaceutically acceptable salt thereof, can be for use in treating cancer. Preferably, the cancer is lung cancer, colorectal cancer, pancreatic cancer, bladder cancer, cervical cancer, endometrial cancer, ovarian cancer, cholangiocarcinoma, or esophageal cancer. More preferably, the cancer is non-small cell lung cancer, pancreatic cancer, or colorectal cancer. Still more preferably, the cancer is non-small cell lung cancer. The cancer can have one or more cancer cells that express the mutant KRas G12D protein. Preferably, the cancer is selected from: KRas G12D mutant non-small cell lung cancer, KRas G12D mutant colorectal cancer, and KRas G12D mutant pancreatic cancer. Additionally, the cancer can be non-small cell lung cancer, and one or more cells express KRas G12D mutant protein. Further, the cancer can be colorectal cancer, and one or more cells express KRas G12D mutant protein. Additionally, the cancer can be pancreatic cancer, and one or more cells express KRas G12D mutant protein. The patient can have a cancer that was determined to have one or more cells expressing the KRas G12D mutant protein prior to administration of the compound or a pharmaceutically acceptable salt thereof. The patient may have been treated with a different course of treatment prior to being treated as described herein.

The compounds provided herein according to any one of Formulae I-IV, or a pharmaceutically acceptable salt thereof, may also be used in the manufacture of a medicament for treating cancer. Preferably, the cancer is lung cancer, colorectal cancer, pancreatic cancer, bladder cancer, cervical cancer, endometrial cancer, ovarian cancer, cholangiocarcinoma, or esophageal cancer. Further preferably, the cancer is non-small cell lung cancer, pancreatic cancer, or colorectal cancer. Still more preferably, the cancer is non-small cell lung cancer. The cancer can have one or more cancer cells that express the mutant KRas G12D protein. When the cancer cells express KRas G12D protein, the cancer can be selected from KRas G12D mutant non-small cell lung cancer, KRas G12D mutant colorectal cancer, and KRas G12D mutant pancreatic cancer.

Also provided herein is a method of treating cancer, comprising administering to a patient in need thereof, an effective amount of a compound according to any one of Formulae I-IV, or a pharmaceutically acceptable salt thereof, and one or more of a PD-1 inhibitor, a PD-L1 inhibitor, a CD4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof, in which the cancer has one or more cells that express a mutant KRas G12D protein. Further provided herein is a compound according to any one of Formulae I-IV, or a pharmaceutically acceptable salt thereof, for use in simultaneous, separate or sequential combination with one or more of a PD-1 or PD-L1 inhibitor, a CD4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof, in the treatment of cancer. Additionally provided is a combination comprising a compound according to any one of Formulae I-IV, or a pharmaceutically acceptable salt thereof, and one or more of a PD-1 or PD-L1 inhibitor, a CD4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof, for simultaneous, separate, or sequential use in the treatment of cancer.

Also provided is a method of treating cancer, comprising administering to a patient in need thereof, an effective amount of a compound according to any one of Formulae I-IV, or a pharmaceutically acceptable salt thereof, and a PD-1 or PD-L1 inhibitor, in which the cancer has one or more cells that express a mutant KRas G12D protein. Further provided is a compound according to any one of Formulae I-IV, or a pharmaceutically acceptable salt thereof, for use in simultaneous, separate or sequential combination with a PD-1 or PD-L1 inhibitor, for use in the treatment of cancer. Additionally provided is a combination comprising a compound according to any one of Formulae I-IV, or a pharmaceutically acceptable salt thereof, and a PD-1 or PD-L1 inhibitor, for simultaneous, separate, or sequential use in the treatment of cancer. As used herein, the PD-1 or PD-L1 inhibitor can be pembrolizunab; the PD-1 or PD-L1 inhibitor can be nivolumab; the PD-1 or PD-L1 inhibitor can be cimiplimab; the PD-1 or PD-L1 inhibitor can be sentilimab; the PD-1 or PD-L1 inhibitor can be atezolizuimab: the PD-1 or PD-L1 inhibitor can be avelumab; the PD-1 or PD-L1 inhibitor can be durvaunmab; or the PD-1 or PD-L1 inhibitor can be lodapilimab. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; or the cancer can be mutant pancreatic cancer in which the cancer has one or more cells that express a KRas G12D mutant protein. The methods described herein also include methods of treating KRas G12D mutant bearing cancers of other origins.

Also provided is a method of treating cancer, comprising administering to a patient in need thereof, an effective amount of a compound according to any one of Formulae I-IV, or a pharmaceutically acceptable salt thereof, and a CDK4/CDK6 inhibitor, or a pharmaceutically acceptable salt thereof, in which the cancer has one or more cells that express a mutant KRas G12D protein. Further provided is a compound according to any one of Formulae I-IV, or a pharmaceutically acceptable salt thereof, for use in simultaneous, separate or sequential combination with a CDK4/CDK6 inhibitor, or a pharmaceutically acceptable salt thereof, for use in the treatment of cancer, in which the cancer has one or more cells that express a mutant KRas G12D protein. Additionally provided is a combination comprising a compound according to any one of Formulae I-IV, or a pharmaceutically acceptable salt thereof, and a CDK4/CDK6 inhibitor, or a pharmaceutically acceptable salt thereof, for simultaneous, separate, or sequential use in the treatment of cancer, in which the cancer has one or more cells that express a mutant KRas G12D protein. As used herein, the CDK4/CDK6 inhibitor can be abemaciclib; the CDK4/CDK6 inhibitor can be Palbociclib; or the CDK4/CDK6 inhibitor can be ribociclib. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. The methods described herein also include methods of treating KRas G12D mutant bearing cancers of other origins.

Also provided is a method of treating cancer, comprising administering to a patient in need thereof, an effective amount of a compound according to any one of Formulae I-IV, or a pharmaceutically acceptable salt thereof, and an EGFR inhibitor, or a pharmaceutically acceptable salt thereof, in which the cancer has one or more cells that express a mutant KRas G12D protein. Further provided is a compound according to any one of Formulae I-IV, or a pharmaceutically acceptable salt thereof, for use in simultaneous, separate or sequential combination with an EGFR inhibitor, or a pharmaceutically acceptable salt thereof, for the treatment of cancer. Additional provided is a combination comprising a compound according to any one of Formulae I-IV, or a pharmaceutically acceptable salt thereof, and an EGFR inhibitor, or a pharmaceutically acceptable salt thereof, for simultaneous, separate, or sequential use in the treatment of cancer. As used herein, the EGFR inhibitor can be erlotinib; the EGFR inhibitor can be afatinib; the EGFR inhibitor can be gefitinib; the EGFR inhibitor can be cetuximab. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; or the cancer can be mutant pancreatic cancer in which the cancer has one or more cells that express a KRas G12D mutant protein. The methods described herein also include methods of treating KRas G12D mutant bearing cancers of other origins.

Also provided is a method of treating cancer, comprising administering to a patient in need thereof, an effective amount of a compound according to any one of Formulae I-IV, or a pharmaceutically acceptable salt thereof, and an ERK inhibitor, or a pharmaceutically acceptable salt thereof, in which the cancer has one or more cells that express a mutant KRas G12D protein. Further provided is a compound according to any one of Formulae I-IV, or a pharmaceutically acceptable salt thereof, for use in simultaneous, separate or sequential combination with an ERK inhibitor, or a pharmaceutically acceptable salt thereof, for the treatment of cancer, in which the cancer has one or more cells that express a mutant KRas G12D protein. Additionally provided is a combination comprising a compound according to any one of Formulae I-IV, or a pharmaceutically acceptable salt thereof, and an ERK inhibitor, or a pharmaceutically acceptable salt thereof, for simultaneous, separate, or sequential use in the treatment of cancer. As used herein, the ERK inhibitor can be LY3214996; the ERK inhibitor can be LTT462; or the ERK inhibitor can be KO-947. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. The methods described herein also include methods of treating KRas G12D mutant bearing cancers of other origins.

Also provided is a method of treating cancer, comprising administering to a patient in need thereof, an effective amount of a compound according to any one of Formulae I-IV, or a pharmaceutically acceptable salt thereof, and an Aurora A inhibitor, in which the cancer has one or more cells that express a mutant KRas G12D protein. Further provided is a compound according to any one of Formulae I-IV, or a pharmaceutically acceptable salt thereof, for use in simultaneous, separate, or sequential combination with an Aurora A inhibitor, or a pharmaceutically acceptable salt thereof, for the treatment of cancer, in which the cancer has one or more cells that express a mutant KRas G12D protein. Additionally provided is a combination comprising a compound according to any one of Formulae I-IV, or a pharmaceutically acceptable salt thereof, and an Aurora A inhibitor, for simultaneous, separate, or sequential use in the treatment of cancer. As used herein, the Aurora A inhibitor can be, but is not limited to, alisertib, tozasertib, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt, and (2R,4R)-1-[3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, or a pharmaceutically acceptable salt thereof. In one embodiment, the Aurora A inhibitor is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid. As described herein the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to any one of Formulae I-IV, or a pharmaceutically acceptable salt thereof, and a SHP2 inhibitor, in which the cancer has one or more cells that express a mutant KRas G12D protein. Further provided is a compound according to any one of Formulae I-IV, or a pharmaceutically acceptable salt thereof, for use in simultaneous, separate, or sequential combination with a SHP2 inhibitor, or a pharmaceutically acceptable salt thereof, for the treatment of cancer, in which the cancer has one or more cells that express a mutant KRas G12D protein. Additionally provided is a combination comprising a compound according to any one of Formulae I-IV, or a pharmaceutically acceptable salt thereof, and a SHP2 inhibitor, for simultaneous, separate, or sequential use in the treatment of cancer. As used herein, the SHP2 inhibitor, or a pharmaceutically acceptable salt thereof, can be a Type I SHP2 Inhibitor or a Type II SHP2 Inhibitor. Examples of Type I SHP2 inhibitors include, but are not limited to, PHPS1, GS-493, NSC-87877, NSC-117199, and Cefsulodin, and pharmaceutically acceptable salts thereof. Examples of Type II SHP2 inhibitors include, but are not limited to, JAB-3068, JAB-3312, RMC-4550, RMC-4630, SHP099, SHP244, SHP389, SHP394, TN0155, RG-6433, and RLY-1971, and pharmaceutically acceptable salts thereof. Additional examples of SHP2 inhibitors include, but are not limited to, BBP-398, IACS-15509, IACS-13909, X37, ERAS-601, SH3809, HBI-2376, ETS-001, and PCC0208023, and pharmaceutically acceptable salts thereof. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to any one of Formulae I-IV, or a pharmaceutically acceptable salt thereof, and a platinum agent, in which the cancer has one or more cells that express a mutant KRas G12D protein. Further provided is a compound according to any one of Formulae I-IV, or a pharmaceutically acceptable salt thereof, for use in simultaneous, separate or sequential combination with a platinum agent, or a pharmaceutically acceptable salt thereof, for the treatment of cancer, in which the cancer has one or more cells that express a mutant KRas G12D protein. Additionally provided is a combination comprising a compound according to any one of Formulae I-IV, or a pharmaceutically acceptable salt thereof, and a platinum agent, for simultaneous, separate, or sequential use in the treatment of cancer. As used herein, the platinum agent can be cisplatin; the platinum agent can be carboplatin: or the platinum agent can be oxaliplatin. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. The methods described herein also include methods of treating KRas G12D mutant bearing cancers of other origins.

Also provided is a method of treating cancer, comprising administering to a patient in need thereof, an effective amount of a compound according to any one of Formulae I-IV, or a pharmaceutically acceptable salt thereof, and pemetrexed, in which the cancer has one or more cells that express a mutant KRas G12D protein. Further provided is a compound according to any one of Formulae I-IV, or a pharmaceutically acceptable salt thereof, for use in simultaneous, separate or sequential combination with pemetrexed, for the treatment of cancer, in which the cancer has one or more cells that express a mutant KRas G12D protein. Additionally provided is a combination comprising a compound according to any one of Formulae I-IV, or a pharmaceutically acceptable salt thereof, and pemetrexed, for simultaneous, separate, or sequential use in the treatment of cancer, in which the cancer has one or more cells that express a mutant KRas G12D protein. As described herein, the cancer has one or more cells that express a KRas G12D mutant protein. Further, a platinum agent can also be administered to the patient (and the platinum agent can be cisplatin, carboplatin, or oxaliplatin). As described herein, the cancer can be colorectal carcinoma in which the cancer has one or more cells that express a KRas G12D mutant protein or the cancer can be mutant pancreatic cancer in which the cancer has one or more cells that express a KRas G12D mutant protein. The methods described herein also include methods of treating KRas G12D mutant bearing cancers of other origins.

The term “pharmaceutically acceptable salt” as used herein refers to a salt of a compound considered to be acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19.

Pharmaceutical compositions containing the compounds of Formulae I-IV as described herein may be prepared using pharmaceutically acceptable additives. The term “pharmaceutically acceptable additive(s)” as used herein for the pharmaceutical compositions, refers to one or more carriers, diluents, and excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., 2012. Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

As used herein, the term “effective amount” refers to an amount that is a dosage, which is effective in treating a disorder or disease, such as a cancerous lesion or progression of abnormal cell growth and/or cell division. The attending physician, as one skilled in the art, can readily determine an effective amount by the use of conventional techniques and by observing results obtained under analogous circumstances. Dosages per day of treatment normally fall within a range of between about 1 mg per day or twice daily and 1000 mg per day or twice daily, more preferably 100 mg per day or twice daily and 900 mg per day or twice daily. Factors considered in the determination of an effective amount or dose of a compound include: whether the compound or its salt will be administered; the co-administration of other agents, if used; the species of patient to be treated; the patient's size, age, and general health; the degree of involvement or stage and/or the severity of the disorder; the response of the individual patient; the mode of administration; the bioavailability characteristics of the preparation administered; the dose regimen selected; and the use of other concomitant medication.

A treating physician, veterinarian, or other medical person will be able to determine an effective amount of the compound for treatment of a patient in need. Preferred pharmaceutical compositions can be formulated as a tablet or capsule for oral administration, a solution for oral administration, or an injectable solution. The tablet, capsule, or solution can include a compound of the present invention in an amount effective for treating a patient in need of treatment for cancer.

As used herein, the terms “treating”, “to treat”, or “treatment”, includes slowing, reducing, or reversing the progression or severity of an existing symptom, disorder, condition, which can include specifically slowing the growth of a cancerous lesion or progression of abnormal cell growth and/or cell division.

As used herein, the term “patient” refers to a mammal in need of treatment. Preferably, the patient is a human that is in need of treatment for cancer, for example, KRas G12D mutant bearing cancers.

Certain abbreviations are defined as follows: “ACN” refers to acetonitrile; AIBN” refers to azobisisobutyronitrile; “Boc-Gly-OH” refers to N-(tert-butoxycarbonyl)glycine; “DCM” refers to dichloromethane; “DIEA” refers to N,N-diisopropyl ethylamine; “(dippf)Rh(cod)BF₄” refers to [1,4-Bis(diphenylphosphino)butane](1,5-cyclooctadiene)rhodium(I) tetrafluoroborate; “DMAP” refers to 4-dimethylaminopyridine; “DMEA” refers to N,N-dimethylethylamine; “DMEM” refers to Dulbecco's modified Eagle's medium; “DMF” refers to N,N-dimethylformamide; “DMSO” refers to dimethylsulfoxide; “DNA” refers to deoxyribonucleic acid; “DPEPhosPdCl₂” refers to dichlorobis(diphenylphophinophenyl)ether palladium (II); “DTT” refers to dithiothreitol; “EDTA” refers to ethylenediaminetetraacetic acid; “EGTA” refers to ethylene glycol-bis(β-aminoethyl ether)-N,N,N′,N′-tetraacetic acid; “ELISA” refers to enzyme-linked immunosorbent assay; “ERK” refers to extracellular signal-regulated kinases; “EtOAc” refers to ethyl acetate; “EtOH” refers to ethanol; “FBS” refers to fetal bovine serum; “GDP” refers to guanosine diphosphate; “GTP” refers to guanosine triphosphate; “HATU” refers to 1-[Bis(dimethylamino)methylene]-1H-1,2,3-triazolo[4,5-b]pyridinium 3-oxide hexafluorophosphate; “HPLC” refers to high-performance liquid chromatography; “HRP” refers to horseradish peroxidase; “IPA” refers to isopropyl alcohol; “IPAm” refers to isopropyl amine; “KOAc” refers to potassium acetate; “LC-ES/MS” refers to liquid chromatograph-electrospray mass spectrometry; “LC-MS” refers to liquid chromatography mass spectrometry; “L-prolinol” refers to [(2S)-pyrrolidin-2yl]methanol; “MAPK” refers to mitogen-activated protein kinases; “mCPBA” refers to 3-chloro-peroxybenzoic acid; “MeOH” refers to methanol; “MTBE” refers to methyl tert-butyl ether; “NaOMe” refers to sodium methoxide; “NBS” refers to N-bromosuccinimide; “NCS” refers to N-chlorosuccinimide; “N-methyl-L-prolinol” refers to [(2S)-1-methylpyrrolidin-2-yl]methanol; “NMP” refers to 1-methylpyrrolidin-2-one; “PCR” refers to polymerase chain reaction; “Pd(dppf)Cl₂” refers to [1,1′-bis(diphenylphosphino)ferrocene]dichloropalladium(II); “RPMI” refers to Roswell Park Memorial Institute; “SCX” refers to strong cation exchange; “SPE” refers to solid phase extraction; SPhos: 2-Dicyclohexylphosphino-2′,6′-dimethoxy-1,1′-biphenyl; “TBDMSCl” refers to tert-butyl dimethyl silyl chloride; “TEA” refers to triethylamine; “TFA” refers to trifluoracetic acid; “THF” refers to tetrahydrofuran; and XPhos: 2-(Dicyclohexylphosphino)-2′,4′,6′-tri-i-propyl-1,1′-biphenyl.

Individual isomers, enantiomers, diastereomers, and atropisomers may be separated or resolved at any convenient point in the synthesis of compounds listed below, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994). The molecules described herein include compounds that are atropisomers and which can exist in different conformations or as different rotomers. Atropisomers are compounds that exist in different conformations arising from restricted rotation about a single bond. Atropisomers can be isolated as separate chemical species if the energy barrier to rotation about the single bond is sufficiently high that the rate of interconversion is slow enough to allow the individual rotomers to be separated from each other. This description is intended to include all of the isomers, enantiomers, diastereomers, and atropisomers possible for the compounds disclosed herein or that could be made using the compounds disclosed herein. In the molecules described herein, only molecules in which the absolute conformation of a chiral center (or atropisomer conformation) is known have used naming conventions or chemical formula that are drawn to indicate the chirality or atropisomerism. Those of skill in the art will readily understand when other chiral centers are present in the molecules described herein and be able to identify the same.

Compounds of any one of Formulae I-IV that are chemically capable of forming salts are readily converted to and may be isolated as a pharmaceutically acceptable salt. Salt formation can occur upon the addition of a pharmaceutically acceptable acid to form the acid addition salt. Salts can also form simultaneously upon deprotection of a nitrogen or oxygen, i.e., removing the protecting group. Examples, reactions and conditions for salt formation can be found in Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

The compounds of the present invention, or salts thereof, may be prepared by a variety of procedures, some of which are illustrated in the Preparations and Examples below. The specific synthetic steps for each of the routes described may be combined in different ways, or in conjunction with steps from different routes, to prepare compounds or salts of the present invention. The products of each step in the Preparations below can be recovered by conventional methods, including extraction, evaporation, precipitation, chromatography, filtration, trituration, and crystallization.

### Preparation 1

### 1-tert-Butyl 2-methyl (2S,3S)-3-[(tert-butyldimethylsilyl)oxy]pyrrolidine-1,2-dicarboxylate

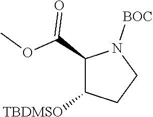 [SMILES: COC(=O)[C@@H]1[C@@H](O[Si](C)(C)C(C)(C)C)CCN1C(=O)OC(C)(C)C]

To a stirred mixture of 1-tert-butyl 2-methyl (2S,3S)-3-hydroxypyrrolidine-1,2-dicarboxylate (500.00 mg, 2.039 mmol, 1.00 eq.) and imidazole (416.33 mg, 6.117 mmol, 3.00 eq.) in DCM (20.00 mL) were added TBDMSCl (768.13 mg, 5.098 mmol, 2.5 eq.) at rt. The resulting mixture was stirred for 2 h. The resulting mixture was concentrated under reduced pressure. The residue was purified by silica gel column chromatography and was eluted with DCM/MeOH/NH₄OH (150:10:1 to 50:10:1) to afford the product (600 mg, 82%) as a white solid. ¹H NMR (300 MHz, DMSO-d6) δ 4.45-4.36 (m, 1H), 3.97-3.86 (m, 1H), 3.66 (d, 3H), 3.48-3.34 (m, 2H), 2.03-1.91 (m, 1H), 1.80-1.69 (m, 1H), 1.36 (d, 9H), 0.86 (s, 9H), 0.07 (s, 6H).

### Preparation 2

### [(2R,3 S)-3-[(tert-Butyldimethylsilyl)oxy]-1-methylpyrrolidin-2-yl]methanol

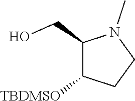 [SMILES: CN1CC[C@H](O[Si](C)(C)C(C)(C)C)[C@H]1CO]

To a stirred solution of 1-tert-butyl 2-methyl (2S,3S)-3-[(tert-butyldimethylsilyl)oxy]pyrrolidine-1,2-dicarboxylate (540 mg, 1.5 mmol, 1.0 eq.) in THE (10 mL) was added LiAlH₄ (4.5 mL, 4.5 mmol, 3.0 eq., 1 M in THF) at 0° C. under N₂ atmosphere. The resulting mixture was stirred overnight at rt. The reaction was quenched with H₂O at rt. The resulting mixture was concentrated under reduced pressure. The residue was purified by silica gel column chromatography and was eluted with DCM/MeOH/NH₄OH (100:10:1˜50:10:1) to afford the product (120 mg, 32.55%) as a white solid. ¹H NMR (300 MHz, DMSO-d6) δ 4.09-3.99 (m, 1H), 3.44-3.25 (m, 2H), 2.86-2.77 (m, 1H), 2.43-2.31 (m, 1H), 2.27 (s, 3H), 2.13-2.06 (m, 1H), 1.90-1.71 (m, 1H), 1.55-1.38 (m, 1H), 0.82 (s, 9H), 0.01 (s, 6H).

### Preparation 3

### [2-(Hydroxymethyl)-1-methylpyrrolidin-2-yl]methanol

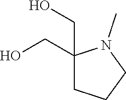

[2-(Hydroxymethyl)pyrrolidin-2-yl]methanol (500.00 mg, 3.812 mmol, 1.00 eq.) and HCHO (343.35 mg, 11.435 mmol, 3.00 eq.) in MeOH (10.00 mL) was stirred for 0.5 h at rt. Then NaBH₃CN (479.07 mg, 7.623 mmol, 2.00 eq.) was slowly added at 0° C. The resulting mixture was stirred for 4 h at rt. The reaction was quenched with H₂O (5 ml) at 0° C. The resulting mixture was diluted with H₂O. The resulting mixture was extracted with EtOAc (200 ml×3). The combined organic layers were washed with sat. aq. NaCl (100 mL×3) and dried over anhydrous Na₂SO₄. After filtration, the filtrate was concentrated under reduced pressure. The residue was purified by silica gel and was eluted with DCM/MeOH/NH₄OH (100:10:1 to 100:20:1) to afford the product (400 mg, 72.3%) as a yellow oil. LC-MS: (ES+H, m/z) [M+H]⁺=146.1. ¹H NMR (400 MHz, DMSO-d6+D2O) δ 3.32-3.19 (m, 4H), 2.66 (t, 2H), 2.25 (s, 3H), 1.62-1.48 (m, 4H).

### Preparations 4 and 5

### tert-Butyl (2S)-2-(1-hydroxyethyl)pyrrolidine-1-carboxylate (isomer 1)

### tert-Butyl (2S)-2-(1-hydroxyethyl)pyrrolidine-1-carboxylate (isomer 2)

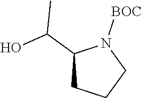

To a solution of tert-butyl (2S)-2-acetylpyrrolidine-1-carboxylate (5.0 g, 23.4 mmol, 1.00 eq.) in THE (100 mL), LiAlH₄ in THE (46.89 mL, 46.89 mmol, 2.00 eq. 1.0 M in THF) was added at 0° C. The resulting mixture was stirred for 3 h at rt. The resulting mixture was quenched with H₂O (3 mL) and concentrated under reduced pressure. The residue was purified with silica gel and was eluted with toluene/acetone (30:1 to 20:1) to afford tert-butyl (S)-2-(1-hydroxyethyl)pyrrolidine-1-carboxylate, Isomer 1 (1.6 g, 31.7%) as a colorless oil and tert-butyl (S)-2-(1-hydroxyethyl)pyrrolidine-1-carboxylate, Isomer 2 (2.0 g, 39.6%) as colorless oil. Isomer 1: ¹H NMR (300 MHz, CDCl₃) δ 3.97-3.87 (m, 2H), 3.56-3.45 (m, 1H), 3.33-3.26 (m, 1H), 2.10-1.94 (m, 1H), 1.88-1.76 (m, 1H), 1.66-1.53 (m, 2H), 1.49 (s, 9H), 1.10 (d, 3H). Isomer 2: ¹H NMR (300 MHz, CDCl₃) δ 3.75-3.72 (m, 2H), 3.51-3.47 (m, 1H), 3.30-3.25 (m, 1H), 2.01-1.90 (m, 1H), 2.04-1.68 (m, 2H), 1.61-1.51 (m, 1H), 1.49 (s, 9H), 1.13 (d, 3H).

### Preparation 6

### 1-[(2S)-1-Methylpyrrolidin-2-yl]ethanol, Isomer 1

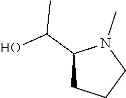

To a stirred mixture of tert-butyl (S)-2-(1-hydroxyethyl)pyrrolidine-1-carboxylate, Isomer 1 (700.00 mg, 3.25 mmol, 1.00 eq.) in THE (200 mL) was added LiAlH₄ (6.5 mL, 6.50 mmol, 2 eq., 1M in THF) dropwise at 0° C. under N₂ atmosphere. Then the mixture was stirred at 70° C. for 2 h. The resulting mixture was quenched with H₂O (3 mL) and was concentrated under reduced pressure. The residue was purified with silica gel and was eluted with DCM/MeOH/NH₄OH (100:5:2 to 90:10:2) to afford the product 1-[(2S)-1-methylpyrrolidin-2-yl]ethanol (300 mg, 71.4%) as colorless oil. ¹H NMR (400 MHz, CDCl₃) δ 3.91-3.87 (m, 1H), 3.19-2.95 (m, 1H), 2.33 (s, 3H), 2.30-2.22 (m, 1H), 2.18-2.06 (m, 1H), 1.83-1.72 (m, 1H), 1.75-1.59 (m, 3H), 1.12 (d, 3H).

### Preparation 7

### 1-[(2S)-1-Methylpyrrolidin-2-yl]ethanol, Isomer 2

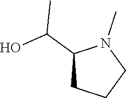

To a stirred solution of tert-butyl (S)-2-(1-hydroxyethyl)pyrrolidine-1-carboxylate, Isomer 2 (600 mg, 2.79 mmol, 1.0 eq.) in THE was added LiAlH₄ (5.57 mL, 5.57 mmol, 2 eq. 1M in THF) dropwise at 0° C. under N₂ atmosphere. Then the mixture was stirred at 70° C. for 2 h. The resulting mixture was quenched with H₂O (3 mL) and concentrated under reduced pressure. The residue was purified with silica gel and was eluted with DCM/MeOH/NH₄OH (100:5:2 to 90:10:2) to afford the product 1-[(2S)-1-methylpyrrolidin-2-yl]ethanol, (220 mg 61.1%) as colorless oil. ¹H NMR (400 MHz, CDCl₃) δ 3.91-3.87 (m, 1H), 3.19-2.95 (m, 1H), 2.33 (s, 3H), 2.30-2.22 (m, 1H), 2.18-2.06 (m, 1H), 1.83-1.72 (m, 1H), 1.75-1.59 (m, 3H), 1.12 (d, 3H).

### Preparation 8

### [(2S)-1,2-Dimethylpyrrolidin-2-yl]methanol

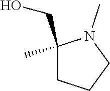

To a stirred solution of tert-butyl (2S)-2-(hydroxymethyl)-2-methylpyrrolidine-1-carboxylate (700.00 mg, 3.251 mmol, 1.00 eq.) in THE (15.00 mL) was added LiAlH₄ (3.90 mL, 3.901 mmol, 1.20 eq., 1 M solution in THF) at 0° C. under N₂ atmosphere. The resulting mixture was stirred for overnight at rt. The reaction was quenched by the addition of H₂O (0.1 mL). The resulting mixture was concentrated under reduced pressure. The residue was purified by silica gel column chromatography and was eluted with DCM/MeOH/NH₃H₂O (150:10:1˜100:10:1) to afford the product (190 mg, 45.23%) as a light-brown oil. ¹H NMR (400 MHz, DMSO-d6) δ 4.21 (s, 1H), 3.18 (s, 2H), 2.84-2.78 (m, 1H), 2.54-2.47 (m, 1H), 2.17 (s, 3H), 1.84-1.76 (m, 1H), 1.67-1.53 (m, 2H), 1.44-1.33 (m, 1H), 0.84 (s, 3H).

### Preparation 9

### tert-Butyl (2S,4S)-2-(hydroxymethyl)-4-methylpyrrolidine-1-carboxylate

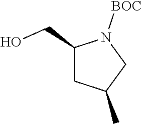

To a stirred mixture of (2S,4S)-1-(tert-butoxycarbonyl)-4-methylpyrrolidine-2-carboxylic acid (2.00 g, 8.72 mmol, 1.0 eq.) in THE (20 mL) was added BH₃-THF (43.62 mL, 43.62 mmol, 5.00 eq., 1.0 M in THF) dropwise at 0° C. under N₂ atmosphere. Then the mixture was stirred overnight. The resulting mixture was quenched with MeOH (8 mL) and concentrated under reduced pressure. The residue was purified with silica gel and was eluted with DCM/MeOH (30:1 to 15:1) to afford the product (1.5 g, 79.8%) as colorless oil. ¹H NMR (400 MHz, CDCl₃) δ 3.90-3.83 (m, 1H), 3.67-3.60 (m, 1H), 3.60-3.56 (m, 1H), 3.51-3.39 (m, 1H), 2.15-1.97 (m, 2H), 1.53-1.44 (m, 1H), 1.40 (s, 9H), 1.35-1.27 (m, 1H), 1.08-0.95 (m, 1H), 0.95 (d, 3H).

### Preparation 10

### [(2S,4S)-1,4-Dimethylpyrrolidin-2-yl]methanol

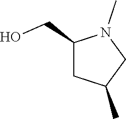

To a stirred mixture of tert-butyl (2S,4S)-2-(hydroxymethyl)-4-methylpyrrolidine-1-carboxylate (1.50 g, 6.97 mmol, 1.00 eq.) in THE (20 mL) was added LiAlH₄ (13.93 mL, 13.93 mmol, 2.00 eq., 1M in THF) dropwise at 0° C. under N₂ atmosphere. The mixture was stirred at 70° C. for 2 h. The resulting mixture was quenched with H₂O (3 mL) and concentrated under reduced pressure. The residue was purified with silica gel and was eluted with DCM/MeOH/NH₄OH (100:5:2 to 90:10:2) to afford the product (600 mg, 66.6%) as colorless oil. ¹H NMR (400 MHz, CDCl₃) δ 3.61-3.57 (m, 1H), 3.37-3.34 (m, 1H), 2.85-2.78 (m, 1H), 2.70-2.60 (m, 1H), 2.46-2.40 (m, 1H), 2.37-2.30 (m, 1H), 2.23 (s, 3H), 2.17-2.07 (m, 1H), 2.05-2.01 (m, 1H), 1.39-1.31 (m, 1H), 0.98 (d, 3H).

### Preparation 11

### [(2S,4R)-1,4-Dimethylpyrrolidin-2-yl]methanol

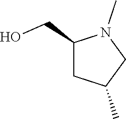

To a stirred mixture of (2S,4R)-1-(tert-butoxycarbonyl)-4-methylpyrrolidine-2-carboxylic acid (300.00 mg, 1.31 mmol, 1.00 eq.) in THE (10.0 mL) was added LiAlH₄ (5.23 mL, 5.23 mmol, 4.0 eq., 1M in THF) dropwise at −20° C. under N₂ atmosphere. The mixture was stirred for 2 h at −20° C., then stirred at 55° C. for another 2 h. The resulting mixture was quenched with MeOH (8 mL) and concentrated under reduced pressure. The residue was purified with silica gel and was eluted with DCM/MeOH/NH₄OH (45:1.5:1 to 45:3:1) to afford the product (90 mg, 53.2%) as a colorless oil. ¹H NMR (400 MHz, CDCl₃) δ 3.58-3.52 (m, 1H), 3.42-3.26 (m, 1H), 3.14-3.05 (m, 1H), 2.48-2.39 (m, 1H), 2.26 (s, 3H), 2.16-2.03 (m, 1H), 1.95-1.80 (m, 2H), 1.46-1.34 (m, 1H), 0.92 (d, 3H).

### Preparation 12

### [(1R,2S,5S)-3-Methyl-3-azabicyclo[3.1.0]hexan-2-yl]methanol

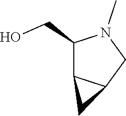

To a stirred mixture of (1R,2S,5S)-3-(tert-butoxycarbonyl)-3-azabicyclo[3.1.0]hexane-2-carboxylic acid (300 mg, 1.32 mmol, 1.0 eq.) in THE (20 mL) were added LiAlH₄ (6.60 mL, 6.60 mmol, 5.0 eq., 1M in THF) dropwise at −40° C. under N₂ atmosphere. The resulting mixture was stirred for 2 h at −40° C. under N₂ atmosphere. Then the resulting mixture was stirred for 4 h at 50° C. under N₂ atmosphere. The reaction was quenched with MeOH (1 mL) at 0° C. The mixture was concentrated under reduced pressure and the resulting residue was purified by silica gel column chromatography and was eluted with DCM/MeOH/NH₄OH (100:10:0.5-100:20:0.5) to afford the product (90 mg, 53.6%) as a colorless oil. LC-MS: (ES+H, m z): [M+H]⁺=128.4. ¹H NMR (400 MHz, CDCl₃) δ 3.78 (dd, 1H), 3.74-3.64 (m, 2H), 3.13 (d, 1H), 2.64-2.57 (m, 1H), 2.53 (dd, 1H), 2.31 (s, 3H), 1.54-1.43 (m, 1H), 1.38-1.29 (m, 1H), 0.85-0.77 (m, 1H), 0.41-0.35 (m, 1H).

### Preparation 13

### [(1S,2S,5R)-3-Methyl-3-azabicyclo[3.1.0]hexan-2-yl]methanol

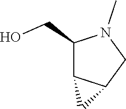

To a stirred mixture of (1S,2S,5R)-3-(tert-butoxycarbonyl)-3-azabicyclo[3.1.0]hexane-2-carboxylic acid (450 mg, 1.98 mmol, 1.0 eq.) in THE (10 mL) was added LiAlH₄ (7.92 mL, 7.92 mmol, 4.0 eq., 1M in THF) dropwise at −20° C. under N₂ atmosphere. The mixture was stirred for 2 h at −20° C., then stirred at 55° C. for another 2 h. The resulting mixture was quenched with MeOH (8 mL) and concentrated under reduced pressure. The residue was purified with silica gel and was eluted with DCM/MeOH/NH₄OH (90:3:0.5 to 90:6:0.5) to afford the product (200 mg, 79.4%) as a colorless oil. ¹H NMR (400 MHz, CDCl₃) δ 3.58-3.52 (m, 1H), 3.45-3.41 (m, 1H), 3.26-3.21m, 1H), 2.64-2.58 (m, 1H), 2.46-2.42 (m, 1H), 2.29 (s, 3H), 1.45-1.39 (m, 1H), 1.35-1.28 (m, 1H), 0.72-0.67 (m, 1H), 0.19-0.16 (m, 1H).

### Preparation 14

### ((2S,4R)-4-Fluoro-1-methylpyrrolidin-2-yl)methanol

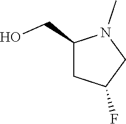

To a stirred solution of 1-(tert-butyl) 2-methyl (2S,4R)-4-fluoropyrrolidine-1,2-dicarboxylate (20.0 g, 80.9 mmol, 1.0 eq.) in THE (200 mL) was added LiAlH₄ (485.3 mL, 485.3 mmol, 6.0 eq., 1M in THF) dropwise at −50° C. under N₂ atmosphere. The resulting mixture was stirred for 1 h at −50° C. under N₂ atmosphere. Then the resulting mixture was stirred for 2 h at 70° C. under N₂ atmosphere. The mixture was allowed to cool to rt. The reaction was quenched by the addition of H₂O at 0° C. The resulting mixture was extracted with EtOAc. The combined organic layers were washed with sat. aq. NaCl and were dried over anhydrous Na₂SO₄. After filtration, the filtrate was concentrated under reduced pressure. The residue was purified by silica gel column chromatography and was eluted with DCM/MeOH (5:1) to afford the product (2.94 g, 27.30%) as a yellow oil. ¹H NMR (400 MHz, CDCl₃) δ 5.22-5.00 (m, 1H), 3.72 (dd, 1H), 3.60-3.41 (m, 2H), 3.10 (s, 1H), 2.84-2.55 (m, 2H), 2.41 (s, 3H), 2.19-2.00 (m, 2H). ¹⁹F NMR (377 MHz, CDCl₃) δ −170.22.

### Preparation 15

### [(2S)-1-[2-(Oxan-2-yloxy)ethyl]pyrrolidin-2-yl]methanol

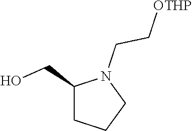

[(2S)-Pyrrolidin-2-yl]methanol (500 mg, 4.94 mmol, 1.0 eq.) and 2-(2-bromoethoxy)oxane (1.09 g, 5.19 mmol, 1.05 eq.) and K₂CO₃ (1.37 g, 9.89 mmol, 2.0 eq.) in ACN (10 mL) were stirred at rt under nitrogen atmosphere. The resulting mixture was stirred for 8 h at 60° C. under N₂ atmosphere. The mixture was diluted with H₂O (100 mL). The resulting mixture was extracted with EtOAc (3×100 ml). The combined organic layers were washed with sat. aq. NaCl (3×50 ml) and were dried over anhydrous Na₂SO₄. After filtration, the filtrate was concentrated under reduced pressure. The residue was purified by silica gel column chromatography and was eluted with (DCM:MeOH=15:1 to 5:1) to afford the product (1.05 g, 93%) as a yellow oil. ¹H NMR (400 MHz, CDCl₃+D₂O) δ 4.63 (dd, 1H), 3.91-3.80 (m, 2H), 3.63-3.59 (m, 1H), 3.54-3.47 (m, 2H), 3.38-3.33 (m, 1H), 3.23 (ddd, 1H), 3.03-2.97 (m, 1H), 2.69 (td, 1H), 2.62-2.55 (m, 1H), 2.39-2.33 (m, 1H), 1.89-1.81 (m, 2H), 1.78-1.69 (m, 4H), 1.61-1.50 (m, 4H).

### Preparation 16

### [(2S)-1-(2-Fluoroethyl)pyrrolidin-2-yl]methanol

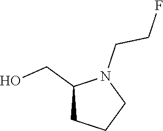

[(2S)-Pyrrolidin-2-yl]methanol (2.015 g, 19.92 mmol) and 1-bromo-2-fluoro-ethane (5.0 g, 39 mmol) were combined in DMEA (6.4 mL, 59 mmol) and were stirred at rt for ˜18 h. The mixture was charged with LiOH (1.00 g, 41.7 mmol), followed by H₂O (0.1 mL) and was stirred at rt for 15 minutes. MeOH (2 mL) was then added followed by DCM (4 mL) and was stirred at rt for 1 h. Then DCM (100 mL) was added, filtered and was concentrated. The residue was purified via silica gel chromatography, eluting with 100% ACN to 90% ACN/10% 2M NH₃ in MeOH to obtain the product (1.78 g, 61%) as a white solid. MS (ES) m/z=148 (M+1).

### Preparation 17

### 6-Bromo-2,3-difluorobenzenemethanol

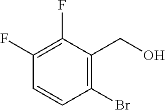 [SMILES: OCc1c(Br)ccc(F)c1F]

6-Bromo-2,3-difluorobenzaldehyde (20.0 g, 88.7 mmol) was dissolved in MeOH (250 mL) and NaBH₄ (6.70 g, 177 mmol) was added in portions. After the exothermic reaction cooled down to ambient temperature (˜1 h), the reaction mixture was poured into sat. aq. NH₄Cl and extracted three times with DCM. The combined organic extracts were washed with H₂O and sat. aq. NaCl, dried over MgSO₄, filtered, and concentrated in vacuo. The residue was dried under high vacuum overnight to give the product (19.5 g, 97%) as a white solid. ¹H NMR (CDCl₃) δ 7.37 (1H, m), 7.07 (1H, m), 4.88 (2H, m), 2.13 (1H, m).

### Preparation 18

### (6-Bromo-2,3-difluorophenyl)methyl methanesulfonate

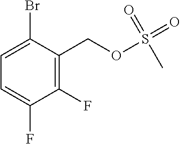

6-Bromo-2,3-difluorobenzenemethanol (19.5 g, 85.7 mmol) was dissolved in THE (200 mL) and DIEA (18.0 mL, 103 mmol) was added. The mixture was cooled to 0° C. and then treated with methanesulfonic anhydride (17.1 g, 94.2 mmol). After stirring at ambient temperature for 18 h, the mixture was diluted with EtOAc:MTBE (1:1) and was washed with cold H₂O. The layers were separated, and the aqueous layer was extracted twice with EtOAc:MTBE (1:1). The combined organics were washed with H₂O and sat. aq. NaCl solution and were dried over MgSO₄ and K₂CO₃, filtered, and concentrated in vacuo to give the product (26.0 g, quantitative) as a yellow oil. ¹H NMR (CDCl₃) δ 7.44 (1H, m), 7.18 (1H, m), 5.43 (2H, d), 3.12 (3H, s).

### Preparation 19

### 2-(6-Bromo-2,3-difluoro-phenyl)acetonitrile

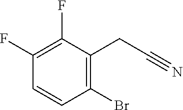 [SMILES: N#CCc1c(Br)ccc(F)c1F]

A mixture of (6-bromo-2,3-difluorophenyl)methyl methanesulfonate (26.0 g, 82.0 mmol) and KCN (6.06 g, 90.3 mmol) in EtOH (200 mL) and H₂O (40.0 mL) was refluxed for 0.5 h and then was cooled to ambient temperature. The solvent was removed in vacuo and the residue was suspended in DCM. The mixture was washed with H₂O, sat. aq. NaHCO₃ and sat. aq. NaCl. The organics were dried over MgSO₄, filtered, and concentrated in vacuo. The crude product was purified by silica gel flash column chromatography and was eluted with 10-100% DCM/hexane to give the product (17.9 g, 94%). ¹H NMR (CDCl₃) δ 7.44 (1H, m), 7.15 (1H, m), 3.91 (2H, d).

### Preparation 20

### Ethyl N-(4-bromo-3-cyano-7-fluoro-benzothiophen-2-yl)carbamate

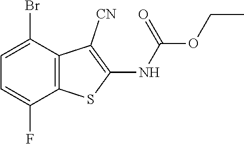 [SMILES: CCOC(=O)Nc1sc2c(F)ccc(Br)c2c1C#N]

A solution of 2-(6-bromo-2,3-difluoro-phenyl)acetonitrile (17.9 g, 77.2 mmol) in DMF (200 mL) was cooled in an ice bath and then was treated with tBuOK (9.30 g, 81.2 mmol) in portions. After addition, the mixture was stirred for 10 minutes and ethoxycarbonyl isothiocyanate (9.80 mL, 81.4 mmol) was added dropwise. The reaction mixture was stirred at ambient temperature for 1 h, and then was heated at 100° C. for 0.5 h. The mixture was then cooled in an ice bath for 10 min. and H₂O (500 mL) was added slowly with stirring. The resultant precipitate was collected by filtration, was rinsed with H₂O and hexanes, and was air dried. The solid was further dried in a vacuum oven at 60° C. overnight to give the product (24.5 g, 84%). ES/MS m/z 340.8 [M−H]⁻.

### Preparation 21

### 2-Amino-4-bromo-7-fluoro-benzothiophene-3-carbonitrile

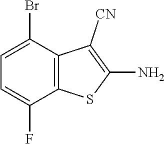 [SMILES: N#Cc1c(N)sc2c(F)ccc(Br)c12]

A mixture of ethyl N-(4-bromo-3-cyano-7-fluoro-benzothiophen-2-yl)carbamate (24.5 g, 71.4 mmol), DMSO (100 mL), and 5N NaOH (80.0 mL, 400 mmol) was refluxed for 4 h. The mixture was cooled to ambient temperature and was treated with cold H₂O while stirring vigorously. The resultant precipitate was collected by filtration, washed with H₂O, and was dried in a vacuum oven at 65° C. overnight to give the product (15.5 g, 80%). ES/MS m/z 268.8 [M−H]⁻.

### Preparation 22

### tert-Butyl N-(4-bromo-3-cyano-7-fluoro-benzothiophen-2-yl)carbamate

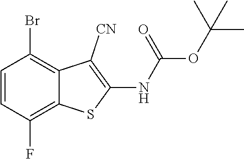 [SMILES: CC(C)(C)OC(=O)Nc1sc2c(F)ccc(Br)c2c1C#N]

A mixture of 2-amino-4-bromo-7-fluoro-benzothiophene-3-carbonitrile (16.0 g, 57.8 mmol) and DIEA (15.0 mL, 86.0 mmol) in DCM (100 mL) and DMF (100 mL) was stirred for 5 minutes at rt. DMAP (700 mg, 5.73 mmol) was added, followed by di-tert-butyldicarbonate (14.5 g, 64.4 mmol), and the resulting mixture was stirred overnight at rt. The solvent was removed in vacuo, and further dried under high vacuum. The residue was treated with 10% aq. citric acid solution (200 mL) and 1:1 EtOAc:MTBE (200 mL). After stirring for 15 min., the resultant precipitate was collected by filtration, washed with H₂O followed by Et₂O, and was dried in a vacuum oven at 60° C. overnight to afford the title compound (17.6 g) as a yellow solid. The layers of the filtrate were separated, and aqueous layer was extracted with 1:1 EtOAc:MTBE (200 mL). The organic layers were combined, washed with sat. aq. NaHCO₃, followed by sat. aq. NaCl solution, dried over MgSO₄, filtered, and concentrated under reduced pressure to afford a crude yellow solid which was recrystallized from EtOAc/hexanes to afford an additional 2 g of the title compound (total product yield 19.6 g, 91%). ES/MS (m z): 370.8 (M+H).

### Preparation 23

### tert-Butyl N-[3-cyano-4-(5,5-dimethyl-1,3,2-dioxaborinan-2-yl)-7-fluoro-benzothiophen-2-yl]carbamate

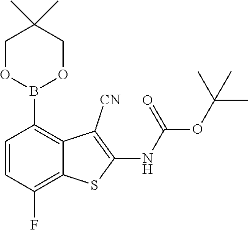 [SMILES: CC1(C)COB(c2ccc(F)c3sc(NC(=O)OC(C)(C)C)c(C#N)c23)OC1]

tert-Butyl N-(4-bromo-3-cyano-7-fluoro-benzothiophen-2-yl)carbamate (16.0 g, 41.4 mmol) and bis(neopentylglycolato)diboron (37.0 g, 157 mmol) were dissolved in 1,4-dioxane (300 mL) under N₂. KOAc (12.2 g, 124 mmol) was added, and the mixture was sparged with N₂ for 1 h at 50° C. DPEPhosPdCl₂ (3.0 g, 4.2 mmol) was added, and the flask was heated at 95° C. for 1 h. The mixture was then cooled to rt, concentrated in vacuo to ˜100 mL, diluted with heptane (200 mL), stirred for 10 min., and filtered through diatomaceous earth rinsing with heptane and heptane:MTBE (1:1). The filtrate was concentrated, dissolved in minimum DCM, and filtered through a pad of silica gel rinsing with EtOAc:heptane (1:1). The filtrate was washed with sat. aq. NH₄Cl and sat. aq. NaCl. The organics were dried over MgSO₄, filtered, and concentrated in vacuo. The residue was purified by silica gel flash column chromatography and was eluted with 5-50% (20% acetone in DCM)/hexane to give the product (13.0 g, 78%). ¹H NMR (DMSO-d6) δ 11.6 (1H, s), 7.61 (1H, m), 7.20 (1H, m), 3.78 (4H, s), 1.54 (9H, s), 1.03 (6H, s).

### Preparation 24

### (6-Bromo-2-fluoro-3-methyl-phenyl)methanol

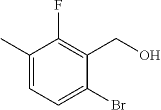

6-Bromo-2-fluoro-3-methyl-benzaldehyde (see US2015/0126449 A1, Example 120A, page 63) was used in a manner analogous to the method of preparation 17 to afford the title compound (13.42 g, 92.2%). ¹H NMR (400.13 MHz, CDCl₃) δ 7.29 (dd, J=8.1, 1.0 Hz, 1H), 7.05 (dd, J=8.1, 8.1 Hz, 1H), 4.87 (d, J=2.2 Hz, 2H), 2.27 (d, J=2.1 Hz, 3H).

### Preparation 25

### (6-Bromo-2-fluoro-3-methyl-phenyl)methyl methanesulfonate

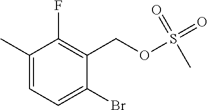

(6-Bromo-2-fluoro-3-methyl-phenyl)methanol was used in a manner essentially analogous to the method of preparation 18 to afford the title compound (19 g, quantitative). ¹H NMR (400.13 MHz, CDCl₃) δ 7.35 (dd, J=8.1, 1.0 Hz, 1H), 7.16 (dd, J=8.1, 8.1, 1H), 5.44 (d, J=2.1 Hz, 2H), 3.10 (s, 3H), 2.29 (d, J=1.9 Hz, 3H).

### Preparation 26

### 2-(6-Bromo-2-fluoro-3-methyl-phenyl)acetonitrile

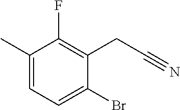

(6-Bromo-2-fluoro-3-methyl-phenyl)methyl methanesulfonate was used in a manner analogous to the method of preparation 19 to afford the title compound (8.45 g, 62.2%). ¹H NMR (400.13 MHz, CDCl₃) δ 7.33 (dd, J=8.1, 1.2 Hz, 1H), 7.11 (dd, J=8.1, 8.1 Hz, 1H), 3.88 (d, J=1.8 Hz, 2H), 2.28 (d, J=2.1 Hz, 3H).

### Preparation 27

### Ethyl N-(4-bromo-3-cyano-7-methyl-benzothiophen-2-yl)carbamate

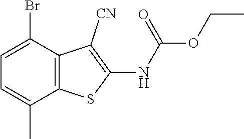 [SMILES: CCOC(=O)Nc1sc2c(C)ccc(Br)c2c1C#N]

A solution of 2-(6-bromo-2-fluoro-3-methyl-phenyl)acetonitrile (6.45 g, 28.3 mmol) in DMF (70 mL) was cooled in an ice-water bath and then was treated with NaH (60% in mineral oil; 1.24 g, 31.1 mmol). The mixture was stirred for 20 min. and ethoxycarbonyl isothiocyanate (3.51 mL, 29.7 mmol) was added. The resulting mixture was heated at 80° C. overnight. The mixture was cooled to rt and was quenched with H₂O. The precipitate was collected by filtration and dried in a vacuum oven at 60° C. overnight. The slightly yellowish-brown solid was diluted with DCM (50 mL), heated to boiling and was sonicated to break up the remaining solid. The resulting white solid was collected by filtration to give the product (2.72 g, 28%). ES/MS (m z): 339.0 (M+H).

### Preparation 28

### 2-Amino-4-bromo-7-methyl-benzothiophene-3-carbonitrile

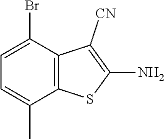

Ethyl N-(4-bromo-3-cyano-7-methyl-benzothiophen-2-yl)carbamate (from preparation 20) was used in a manner analogous to the method of preparation 21, except the reaction was heated at 100° C. for two days, to afford the title compound (1.9 g, 90%). ES/MS (m z): 267.0 (M+H).

### Preparation 29

### tert-Butyl N-(4-bromo-3-cyano-7-methyl-benzothiophen-2-yl)carbamate

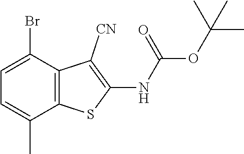 [SMILES: Cc1ccc(Br)c2c(C#N)c(NC(=O)OC(C)(C)C)sc12]

2-Amino-4-bromo-7-methyl-benzothiophene-3-carbonitrile (from preparation 21) was used in a manner similar to the method of preparation 22, using THE as the reaction solvent, to afford the title compound (1.7 g, 64%). ES/MS (m z): 367.0 (M+H).

### Preparation 30

### tert-Butyl N-[3-cyano-4-(5,5-dimethyl-1,3,2-dioxaborinan-2-yl)-7-methyl-benzothiophen-2-yl]carbamate

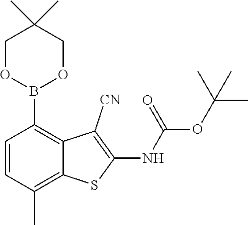 [SMILES: Cc1ccc(B2OCC(C)(C)CO2)c2c(C#N)c(NC(=O)OC(C)(C)C)sc12]

A mixture of tert-Butyl N-(4-bromo-3-cyano-7-methyl-benzothiophen-2-yl)carbamate (1.60 g, 4.36 mmol) and KOAc (1.07 g, 10.9 mmol) in toluene (60 mL) was heated in a 175° C. reaction block to remove any H₂O into a Dean-Starke trap. The solution was cooled to 50° C., treated with bis(neopentylglycolato)diboron (1.25 g, 5.53 mmol) and bis(triphenylphosphine)palladium(II) dichloride (0.153 g, 0.218 mmol), and heated to 80° C. overnight. The reaction mixture was diluted with EtOAc, stirred for 10 min. and was filtered through diatomaceous earth. The filtrate was washed twice with sat. aq. NaHCO₃ followed by sat. aq. NaCl, dried over MgSO₄, filtered, and was concentrated in vacuo. The residue was purified by silica gel flash column chromatography and was eluted with 10-40% (20% acetone in DCM)/hexane to give the product as a white solid (1.02 g, 58.5%). ES/MS (m z): 277.0 (M-tBu-CH₂C(Me)₂CH₂+H). ¹H NMR (400.13 MHz, DMSO-d6): δ 11.27 (s, 1H), 7.49 (d, J=7.2 Hz, 1H), 7.16 (d, J=7.2 Hz, 1H), 3.77 (s, 4H), 3.32 (s, 1H), 2.49 (s, 3H), 1.54 (s, 9H), 1.03 (s, 6H).

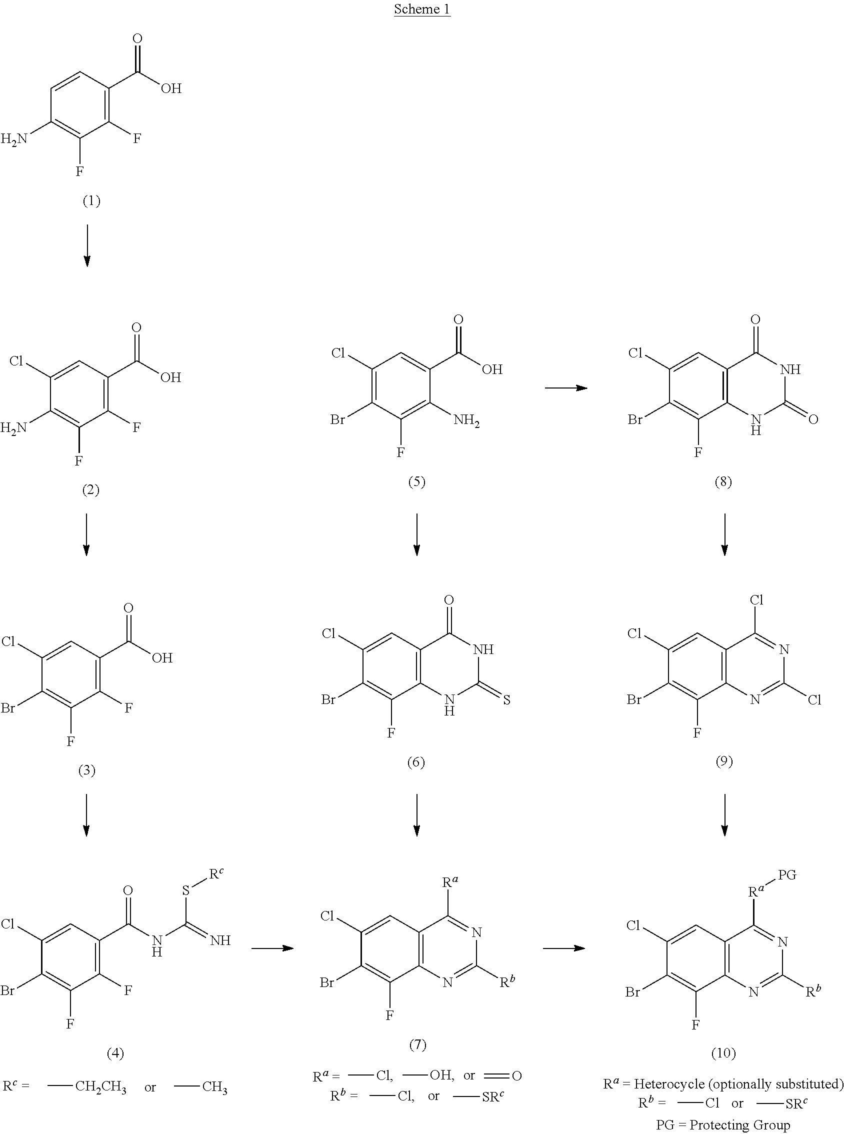

Scheme 1 depicts the preparation of quinazoline compounds (10). Using well-known conditions, commercially available 4-amino-2,3-difluoro-benzoic acid (1) may be chlorinated with a variety of suitable reagents such as, but not limited to, NCS, SO₂Cl₂, Cl₂, and 1,3-dichloro-5,5-dimethylhydantoin, to furnish a chlorinated benzoic acid (2). Subjecting the chlorinated benzoic acid (2) to typical Sandmeyer conditions known to one of skill in the art, provides 4-bromo-5-chloro-2,3-difluoro-benzoic acid (3). 4-bromo-5-chloro-2,3-difluoro-benzoic acid (3) may be treated with an alkylated thiourea, or a suitable salt thereof, to afford an aryl sulfanylcarbonimidoyl (4). Subsequent annulation of the aryl sulfanylcarbonimidoyl (4) may be accomplished with heat in an appropriate polar aprotic solvent to give quinazoline (7) which a person skilled in the art will recognize may alternatively be synthesized starting from commercially available 2-amino-4-bromo-3-fluoro-benzoic acid, chlorinating under the previously described conditions to supply 2-amino-4-bromo-5-chloro-3-fluoro-benzoic acid (5). 2-Amino-4-bromo-5-chloro-3-fluoro-benzoic acid (5) may be cyclized to quinazoline (6) by forming the corresponding acid chloride followed by addition of ammonium thiocyanate. 2-Thioxo quinazoline-4-one (6) may be converted to the corresponding alkylated quinazoline sulfide (7) under basic conditions and addition of a suitable alkyl electrophile. A number of apt protecting groups may be appended to the quinazoline (7) to provide protected quinazoline (10). 2-Amino-4-bromo-5-chloro-3-fluoro-benzoic acid (5) may also be employed to furnish quinazoline-2,4-dione (8) en route to quinazoline (10) by addition of urea and under heat. One of skill in the art will recognize that a dione (8) may be chlorinated by use of phosphoryl chloride or a similar chlorinating reagent. Chlorines adjacent to the nitrogen atoms on the quinazoline may be selectively displaced to provide substituted the quinazoline (10).

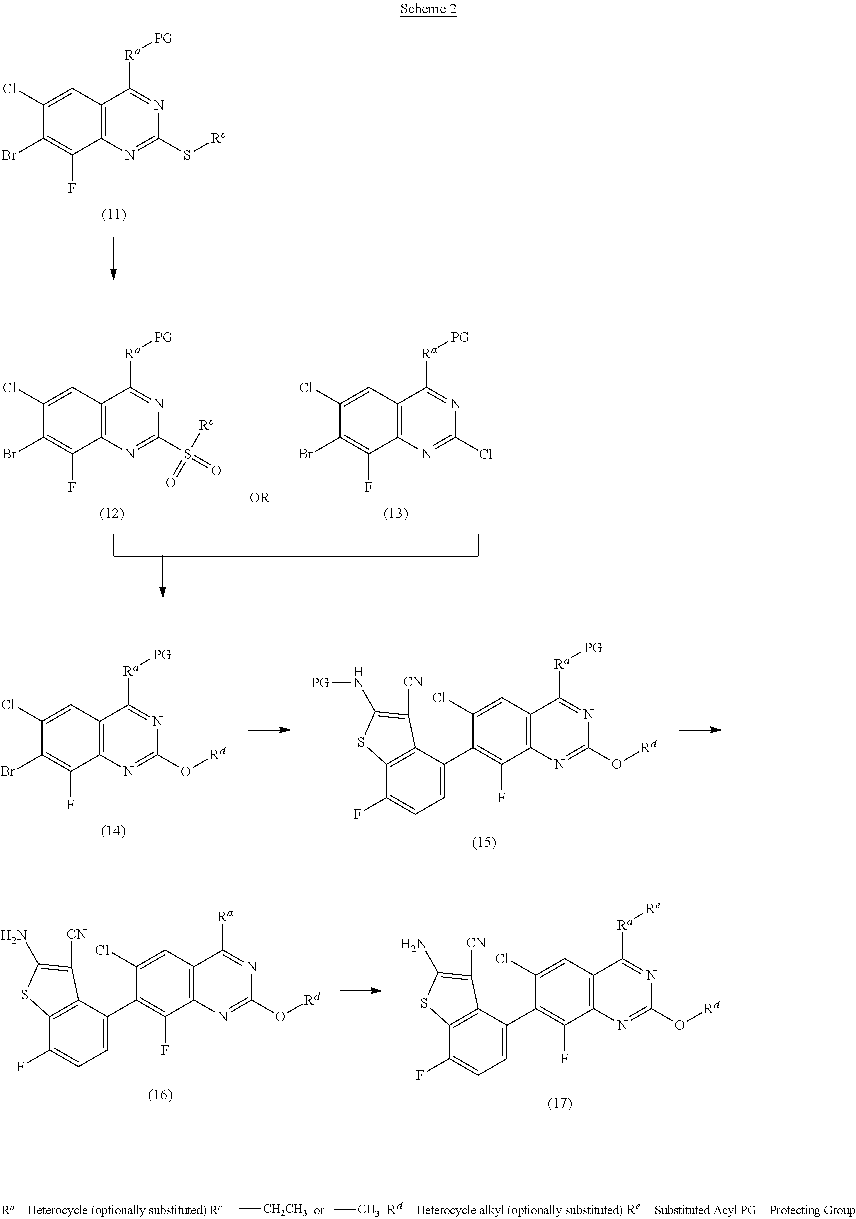

Scheme 2 depicts the preparation of benzothiophene-substituted quinazoline compounds (17). A thioether (11) may be oxidized with mCPBA in DCM or other suitable oxidizing agent to furnish a sulfone (12). Nucleophilic aromatic substitution (commonly known as S_{N}Ar) of the sulfone moiety using a strong non-nucleophilic base in a polar aprotic solvent such as THF and a variety of heterocyclylalkyl alcohols gives a substituted quinazoline (14). Alternatively, the S_{N}Ar may be achieved by heating an aryl chloride (13) with the aforementioned alcohols and stoichiometric amounts of KF in DMSO. Aryl coupling of the bromo-quinazoline (14) with a benzothiophene boronate ester may be achieved to give a bis-aryl compound (15) under Suzuki conditions using a base such as Cs₂CO₃ and a variety of palladium (II) complexes of which the bis(2-(diphenylphosphino)phenyl)ether ligand is well known to those of skill in the art. Subsequent removal of the protecting group(s) may be achieved by methods appropriate to the protecting group used such as BOC removal by TFA in DCM. The heterocyclic group on quinazoline (16) may be acylated under typical amide coupling reagents such a HATU, polar aprotic solvent such as DMF and a non-nucleophilic base to give the amide (17).

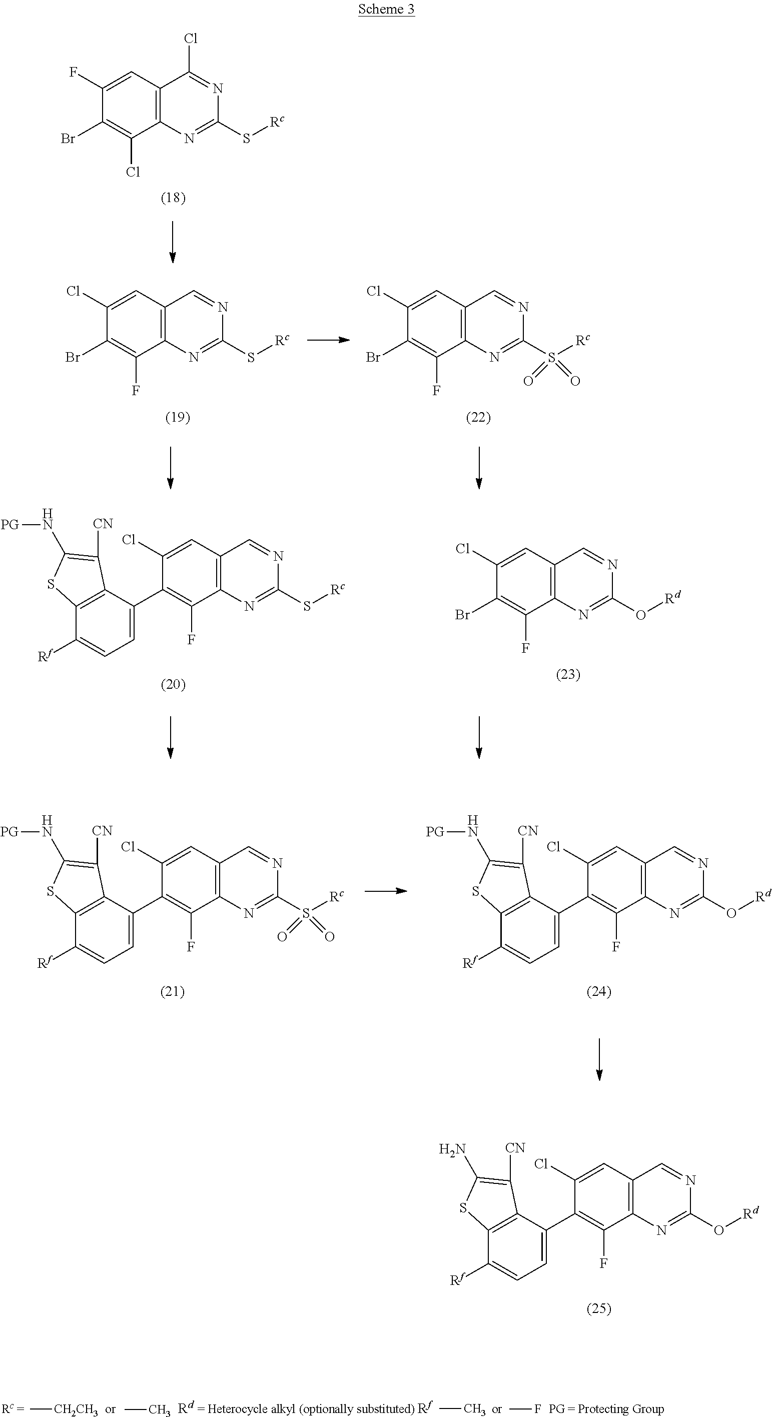

Scheme 3 depicts the preparation of the 2,7-substituted quinazoline compounds (25). A chloro-quinazoline (18) may be de-chlorinated by using a suitable palladium-ligand complex, such the bis(diphenylphosphino)ferrocene ligand, and NaBH₃CN plus a base such as N,N,N′,N′-tetramethylethylenediamine to form the hydrido-substituted quinazoline (19). The quinazoline (19) may be used convergently in two synthetic routes to obtain access to different substitution points. Suzuki coupling of the bromo-quinazoline (19) gives a bis-aryl (20) which may be of oxidized at the thioether moiety to yield a sulfone (21). This then sets up an S_{N}Ar reaction for the introduction of the ether moiety to the quinazoline (24). Alternatively, the oxidation of a thioether (19) may be conducted directly to allow for the S_{N}Ar introduction of the heterocyclylalkyl alcohol piece to give various quinazolines (23). Then, a Suzuki aryl coupling gives bis-aryl compounds (24) which represent the convergence of the two routes which then may be deprotected to yield substituted quinazolines (25).

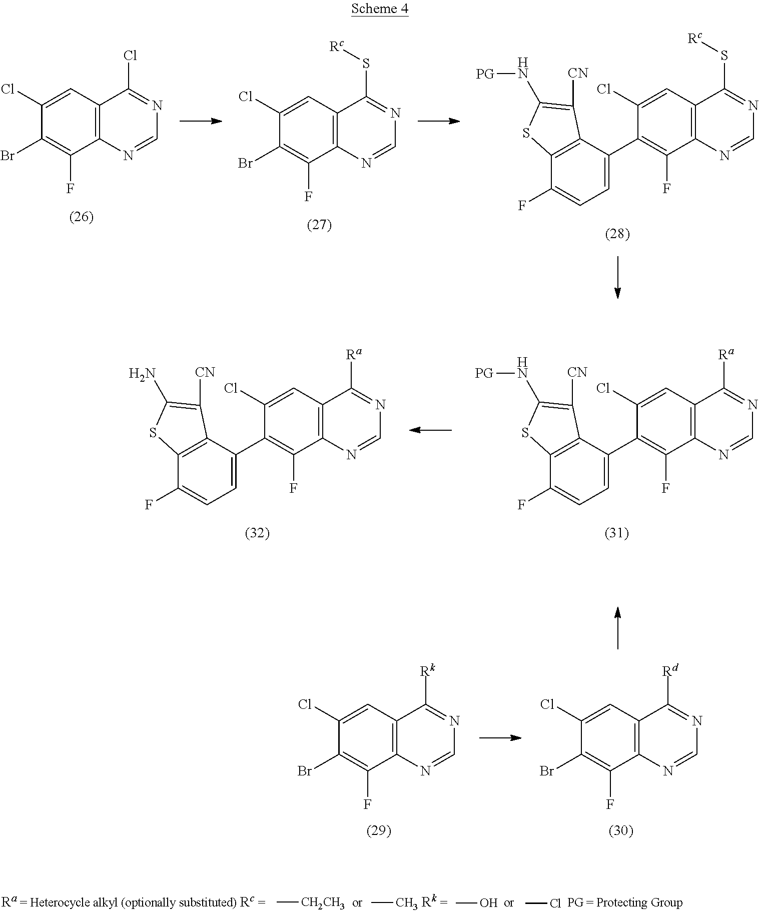

Scheme 4 depicts the preparation of the quinazoline compounds (32). Nucleophilic displacement at the C-4 center of chloro-quinazolines (26) by the appropriate substituted sulfide gives a thioether (27). A Suzuki coupling of the bromo-quinazoline (27) and the substituted benzothiophene boronate ester gives substituted quinazoline compounds (28) which may be substituted by an appropriate heterocyclic nucleophile gives further substituted quinazolines (31). Alternatively, a 4,7 disubstituted quinazoline (31) may be constructed from either a 4-chloro or a 4-hydroxy quinazoline (29) via nucleophilic substitution to install the appropriate heterocycle to the quinazoline (30). Similar palladium-catalyzed Suzuki-Miyaura coupling conditions may be employed to give a protected bis-aryl (31). As previously detailed, a protected aminothiophene (31) may be deprotected under a variety of conditions well known to one of skill in the art.

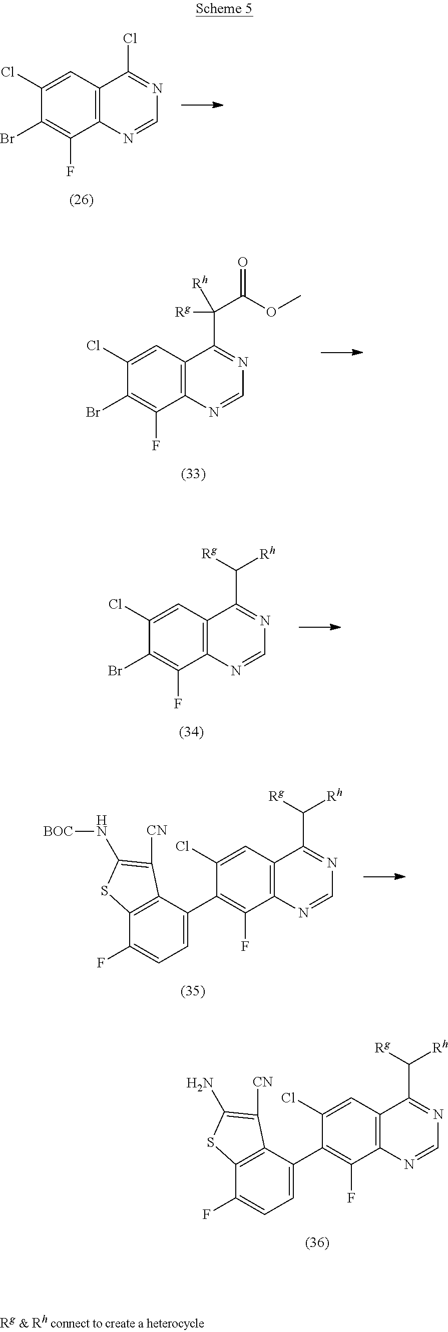

Scheme 5 depicts the synthesis of the compounds of (36). Previously described 4-chloroquinazoline (26) may be selectively coupled with an appropriate dicarboxylate using a suitable non-nucleophilic base such as lithium bis(trimethylsilyl)amide, lithium diisopropylamide, or potassium bis(trimethylsilyl)amide to provide a quaternary methyl acetate (33). One of ordinary skill in the art will recognize that the quinazoline ester (33) may be decarboxylated under a variety of conditions such as metal catalysis, photoredox catalysis, or Krapcho conditions utilizing a suitable polar aprotic solvent, inorganic salt and heat to furnish methine quinazoline (34). Palladium-catalyzed Suzuki-Miyaura coupling conditions may be employed on the 7-bromoquinazoline (34) to affect an aryl-aryl bond formation, generating a substituted quinazoline (35). Additionally, as previously detailed, protected aminothiophene (35) may be deprotected under a variety of conditions well known to one of skill in the art.

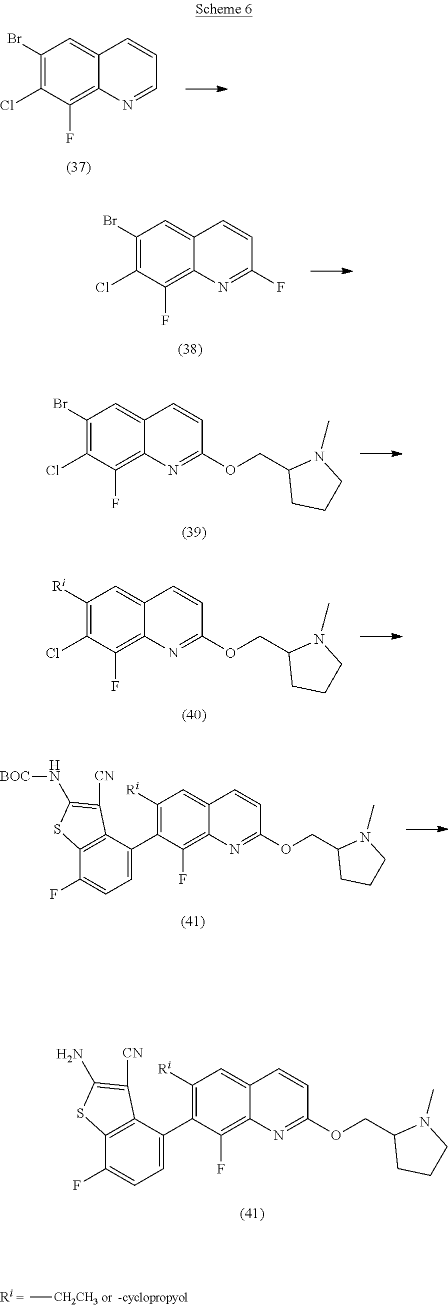

Scheme 6 depicts the synthesis of compound 42. Commercially available 6-bromo-7-chloro-8-fluoro-quinoline (37) may be oxidized with AgF₂ in a Chichibabin-type process to afford 6-bromo-7-chloro-8-fluoro-quinoline (38). One of skill in the art will recognize that addition of α-fluorinated quinoline (38) to a solution of N-methyl-L-prolinol and suitable non-nucleophilic base such as lithium bis(trimethylsilyl)amide, lithium diisopropylamide, or potassium bis(trimethylsilyl)amide provides substituted quinoline (39). Alkyl groups may be selectively substituted on 6-bromo quinoline (39) to provide alkylated quinoline (40) using typical palladium-catalyzed Suzuki-Miyaura coupling conditions. As previously mentioned, similar conditions may be further employed on 7-chloroquinoline (40) to affect an aryl-aryl bond formation, to generate bis-aryl (41). Additionally, as previously detailed, protected aminothiophene (41) may be deprotected under a variety of conditions well known to one of skill in the art.

### Preparation 31

### 4-amino-5-chloro-2,3-difluoro-benzoic acid

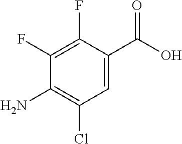 [SMILES: Nc1c(Cl)cc(C(=O)O)c(F)c1F]

A solution of 4-amino-2,3-difluoro-benzoic acid (33.3 g, 192 mmol) in ACN (400 mL) was charged with NCS (34.5 g, 251 mmol, 1.30 eq.) in several portions. The reaction was heated at 80° C. for 1.5 h. The flask was placed in an ice bath and when temperature reached ˜10° C., 1.2 L (3 volumes) of H₂O was added dropwise over ˜1 h. The solids were filtered and rinsed with 500 mL H₂O and left under vacuum to dry (batch 1). The filtrate was further extracted with EtOAc (2×1 L), was dried over anhydrous Na₂SO₄, filtered and was concentrated to a solid. The solids were slurried with H₂O (1 L) for 2 h, then were filtered and dried under vacuum (batch 2). The batches were combined to give the product (25.9 g, 65%) as a tan solid. MS (ES) m/z=162 (M-CO2H). ¹H NMR (399.80 MHz, DMSO-d6): δ 7.88 (dd, J=2.2, 6.2 Hz, 1H).

### Preparation 32

### 4-bromo-5-chloro-2,3-difluoro-benzoic acid

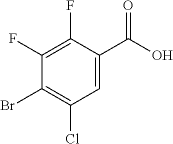 [SMILES: O=C(O)c1cc(Cl)c(Br)c(F)c1F]

A suspension of ACN (200 mL) and CuBr₂ (25.8 g, 116 mmol, 2.00 eq) was placed in a heating mantle and the temperature controller was turned on to 78° C. As the mixture was heated, tert-butyl nitrite (30 mL, 227 mmol, 3.9 eq) was added drop wise over 10 min. The mixture was heated for 15 min. at 78° C. then 4-amino-5-chloro-2,3-difluoro-benzoic acid (12.00 g, 57.81 mmol) was added in several portions. The mixture was heated at 78° C. for ˜7 hrs. The mixture was concentrated, EtOAc (200 mL) was added, and it was washed with 1N HCl (2×100 mL), sat. aq. NaHSO₃ (100 mL) and sat. aq. NaCl (100 mL). The organics were dried over anhydrous Na₂SO₄ which was then filtered, concentrated and dried under house vacuum at rt to afford the product (14 g, 89%) as a tan solid. MS (ES−) m/z=262 (M-CO₂H). ¹H NMR (399.80 MHz, DMSO-d6): δ 7.88 (dd, J=2.2, 6.2 Hz, 1H),

### Preparation 33

### 4-bromo-5-chloro-N-(ethylsulfanylcarbonimidoyl)-2,3-difluoro-benzamide

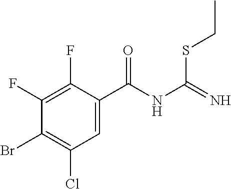 [SMILES: CCSC(=N)NC(=O)c1cc(Cl)c(Br)c(F)c1F]

A suspension of 4-bromo-5-chloro-2,3-difluoro-benzoic acid (2.0 g, 6.1 mmol) in ACN (30 mL) was charged with S-ethylisothiourea hydrobromide (2.11 g, 11.2 mmol, 1.8 eq.), DIEA (2.6 mL, 15 mmol, 2.4 eq.), HATU (4.33 g, 11.2 mmol, 1.8 eq.) and was stirred at rt for 15 min. The precipitate was filtered. The filtrate was treated dropwise with H₂O (120 mL) and was stirred at rt for 15 min. Solids were filtered and were left under house vacuum at 50° C. to obtain the product (1.86 g, 75%) as a pale orange solid. MS (ES+) m/z=359 (M+1).

### Preparation 34

### 7-bromo-6-chloro-2-ethylsulfanyl-8-fluoro-3H-quinazolin-4-one

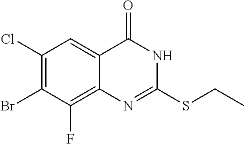 [SMILES: CCSc1nc2c(F)c(Br)c(Cl)cc2c(=O)[nH]1]

A solution of 4-bromo-5-chloro-N-(ethylsulfanylcarbonimidoyl)-2,3-difluoro-benzamide (8.6 g, 24 mmol) in NMP (80 mL) was heated at 100° C. for 6 h. The mixture was cooled to rt and was treated with 240 mL H₂O drop wise and was stirred at rt for 2 h. Solids were filtered and were dried under vacuum at 50° C. The solids were stirred with 40 mL DCM for 0.5 h and were filtered to obtain the product (5.22 g, 64%). MS (ES+) m/z=338 (M+1).

### Preparation 35

### 7-bromo-4,6-dichloro-2-ethylsulfanyl-8-fluoro-quinazoline

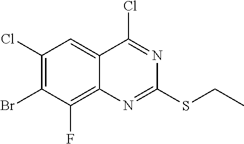 [SMILES: CCSc1nc(Cl)c2cc(Cl)c(Br)c(F)c2n1]

A suspension of 7-bromo-6-chloro-2-ethylsulfanyl-8-fluoro-3H-quinazolin-4-one (5.2 g, 15 mmol) in DCM (75 mL) was charged with (chloromethylene)dimethyliminium chloride (7.96 g, 31.1 mmol, 4.0 eq.) and was stirred at rt for 18 h. The reaction mixture was poured into 100 mL H₂O, partitioned, and was washed with sat. aq. NaCl. The organics were dried over anhydrous Na₂SO₄, filtered and was concentrated. The residue was purified via silica gel chromatography, eluting with a gradient of 10% to 60% DCM in hexanes to obtain the product (5.1 g, 93%) as a white solid. MS (ES+) m/z=338 (M+1).

### Preparation 35a

### tert-butyl 9-(7-bromo-6-chloro-2-ethylsulfanyl-8-fluoro-quinazolin-4-yl)-3-oxa-7,9-diazabicyclo[3.3.1]nonane-7-carboxylate

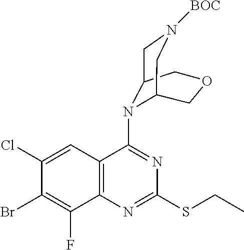 [SMILES: CCSc1nc(N2C3COCC2CN(C(=O)OC(C)(C)C)C3)c2cc(Cl)c(Br)c(F)c2n1]

7-bromo-4,6-dichloro-2-ethylsulfanyl-8-fluoro-quinazoline (0.34 g, 0.96 mmol) was placed in a vial with tert-butyl 3-oxa-7,9-diazabicyclo[3.3.1]nonane-7-carboxylate (0.25 g, 1.0 mmol, 1.1 eq.) and DIPEA (0.33 mL, 1.9 mmol, 2 eq.) in ACN (5 mL) and DMF (2 mL) and was stirred at rt for 2 h. The mixture was diluted with EtOAc and was washed with H₂O and sat. aq. NaCl. The organic layer was dried over anhydrous Na₂SO₄, filtered and was concentrated to an oil. The oil was purified via silica gel chromatography eluting with a gradient from 100% hexane to 30% EtOAc in hexane to obtain the product (0.483 g, 92%) as a tan solid. MS (ES) m/z=547/549 (M+1).

### Preparation 36

### tert-Butyl 9-[7-[2-(tert-butoxycarbonylamino)-3-cyano-7-fluoro-benzothiophen-4-yl]-6-chloro-2-ethylsulfanyl-8-fluoro-quinazolin-4-yl]-3-oxa-7,9-diazabicyclo[3.3.1]nonane-7-carboxylate

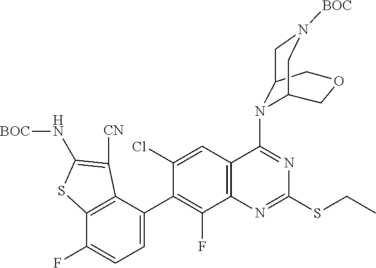 [SMILES: CCSc1nc(N2C3COCC2CN(C(=O)OC(C)(C)C)C3)c2cc(Cl)c(-c3ccc(F)c4sc(NC(=O)OC(C)(C)C)c(C#N)c34)c(F)c2n1]

7,9-Diazabicyclo[3.3.1]nonane-7-carboxylate (0.480 g, 0.8761 mmol) was combined in a 25 mL vial with tert-butyl N-[3-cyano-4-(5,5-dimethyl-1,3,2-dioxaborinan-2-yl)-7-fluoro-benzothiophen-2-yl]carbamate (0.460 g, 1.14 mmol), Cs₂CO₃ (0.874 g, 2.6 mmol) and dichloro[bis(2-(diphenylphosphino)phenyl)ether]palladium(II) (0.128 g, 0.17 mmol) in toluene (11 mL). The vial was capped and purged with alternating N₂/vacuum (2×). The vial was placed in heating block and was heated at 120° C. for 2 h. The reaction was diluted with EtOAc and was filtered over diatomaceous earth and was rinsed with EtOAc. The filtrate was concentrated and purified with a gradient from 100% hexane to 40% EtOAc in hexane to afford the product (0.385 g, 58%) as an orange gummy solid. MS (ES) m/z=760 (M+1).

### Preparation 37

### tert-Butyl 9-[7-[2-(tert-butoxycarbonylamino)-3-cyano-7-fluoro-benzothiophen-4-yl]-6-chloro-2-ethylsulfonyl-8-fluoro-quinazolin-4-yl]-3-oxa-7,9-diazabicyclo[3.3.1]nonane-7-carboxylate

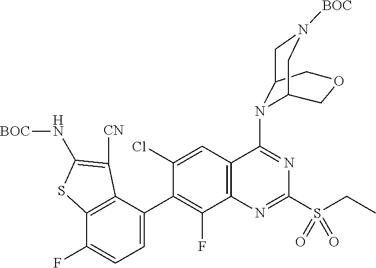 [SMILES: CCS(=O)(=O)c1nc(N2C3COCC2CN(C(=O)OC(C)(C)C)C3)c2cc(Cl)c(-c3ccc(F)c4sc(NC(=O)OC(C)(C)C)c(C#N)c34)c(F)c2n1]

tert-Butyl 9-[7-[2-(tert-butoxycarbonylamino)-3-cyano-7-fluoro-benzothiophen-4-yl]-6-chloro-2-ethylsulfanyl-8-fluoro-quinazolin-4-yl]-3-oxa-7,9-diazabicyclo[3.3.1]nonane-7-carboxylate (0.385 g, 0.51 mmol) and mCPBA (0.220 g, 1.27 mmol) were stirred in DCM (10 mL) at rt for 1 h. The reaction was diluted with DCM and was washed with H₂O and Na₂S₂O₃, dried over anhydrous Na₂SO₄, filtered and concentrated to an oil. The oil was purified by silica gel chromatography, eluting with a gradient from 100% hexane to 60% EtOAc in hexane to obtain the product (0.205 g, 32%). MS (ES) m/z=792 (M+1).

### Preparation 38

### tert-Butyl 9-[7-[2-(tert-butoxycarbonylamino)-3-cyano-7-fluoro-benzothiophen-4-yl]-6-chloro-8-fluoro-2-[[(2S)-1-methylpyrrolidin-2-yl]methoxy]quinazolin-4-yl]-3-oxa-7,9-diazabicyclo[3.3.1]nonane-7-carboxylate

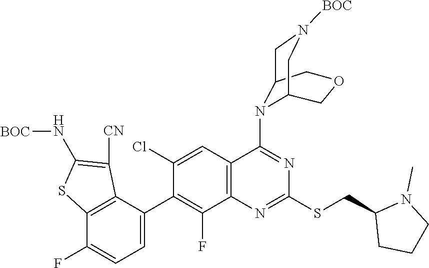 [SMILES: CN1CCC[C@H]1CSc1nc(N2C3COCC2CN(C(=O)OC(C)(C)C)C3)c2cc(Cl)c(-c3ccc(F)c4sc(NC(=O)OC(C)(C)C)c(C#N)c34)c(F)c2n1]

Lithium bis(trimethylsilyl)amide (1 mol/L) in THF was added to a solution of N-methyl-L-prolinol (0.02 mL, 0.2 mmol) and stirred for 5 min. tert-Butyl 9-[7-[2-(tert-butoxycarbonylamino)-3-cyano-7-fluoro-benzothiophen-4-yl]-6-chloro-2-ethylsulfonyl-8-fluoro-quinazolin-4-yl]-3-oxa-7,9-diazabicyclo[3.3.1]nonane-7-carboxylate (0.060 g, 0.076 mmol) in THE (0.5 mL was) added and was stirred at rt for 0.5 h. The mixture was diluted with EtOAc and was concentrated to afford the crude product (61 mg, quantitative) as an oil.

### Example 1

### 2-amino-4-[6-chloro-8-fluoro-2-[[(2S)-1-methylpyrrolidin-2-yl]methoxy]-4-(3-oxa-7,9-diazabicyclo[3.3.1]nonan-9-yl)quinazolin-7-yl]-7-fluoro-benzothiophene-3-carbonitrile

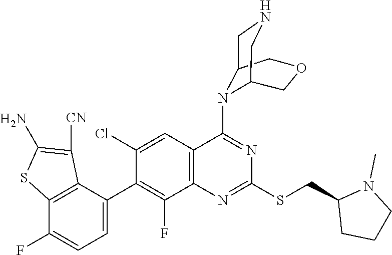 [SMILES: CN1CCC[C@H]1CSc1nc(N2C3CNCC2COC3)c2cc(Cl)c(-c3ccc(F)c4sc(N)c(C#N)c34)c(F)c2n1]

tert-Butyl 9-[7-[2-(tert-butoxycarbonylamino)-3-cyano-7-fluoro-benzothiophen-4-yl]-6-chloro-8-fluoro-2-[[(2S)-1-methylpyrrolidin-2-yl]methoxy]quinazolin-4-yl]-3-oxa-7,9-diazabicyclo[3.3.1]nonane-7-carboxylate (0.060 g, 0.074 mmol) was stirred in DCM (2 mL) and TFA (1 mL) at rt for 1 h. The mixture was concentrated, and the residue was purified via silica gel chromatography, eluting with a gradient of 100% DCM to 10% 2N NH₃ in MeOH in DCM to obtain the product (0.024 g, 53%) as a white solid. MS (ES) m/z=612 (M+1).

The Example compounds in Table 3 were prepared in a similar manner as described for Example, using the appropriate piperazine derivative at C4 and the appropriate alcohol at C2. Various methods were used to purify the compounds, which would be apparent to one skilled in the art.

TABLE 3
Example Compounds 2 to 9.

| | | | MS |
|---|---|---|---|
| | | | (ES) m/z |
| Example | Chemical Name | Structure | (M + H) |
| 2 | 2-amino-4-[6-chloro-8-fluoro- 2-[[(2S)-1-(2- fluoroethyl)pyrrolidin-2- yl]methoxy]-4-(3-oxa-7,9- diazabicyclo[3.3.1]nonan-9- yl)quinazolin-7-yl]-7-fluoro- benzothiophene-3-carbonitrile | 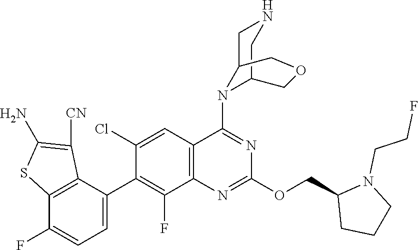 [SMILES: N#Cc1c(N)sc2c(F)ccc(-c3c(Cl)cc4c(N5C6CNCC5COC6)nc(OC[C@@H]5CCCN5CCF)nc4c3F)c12] | 644 |
| 3 | 2-amino-4-[6-chloro-4-(3,8- diazabicyclo[3.2.1]octan-3- yl)-8-fluoro-2-[[(2S)-1- methylpyrrolidin-2- yl]methoxy]quinazolin-7-yl]- 7-fluoro-benzothiophene-3- carbonitrile (Isomer 1) | 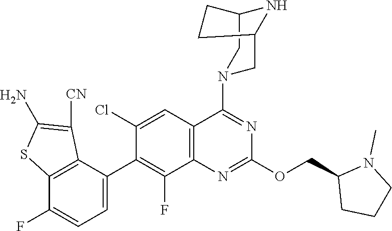 [SMILES: CN1CCC[C@H]1COc1nc(N2CC3CCC(C2)N3)c2cc(Cl)c(-c3ccc(F)c4sc(N)c(C#N)c34)c(F)c2n1] | 596 |
| 4 | 2-amino-4-[6-chloro-4-(3,8- diazabicyclo[3.2.1]octan-3- yl)-8-fluoro-2-[[(2S,4R)-4- fluoro-1-methyl-pyrrolidin-2- yl]methoxy]quinazolin-7-yl]- 7-fluoro-benzothiophene-3- carbonitrile (Isomer 2) | 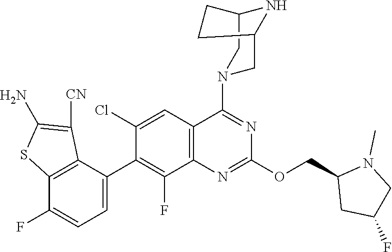 [SMILES: CN1C[C@H](F)C[C@H]1COc1nc(N2CC3CCC(C2)N3)c2cc(Cl)c(-c3ccc(F)c4sc(N)c(C#N)c34)c(F)c2n1] | 614 |
| 5 | 2-amino-4-[6-chloro-4-(3,8- diazabicyclo[3.2.1]octan-3- yl)-8-fluoro-2-[[(2S,4R)-4- fluoro-1-methyl-pyrrolidin-2- yl]methoxy]quinazolin-7-yl]- 7-fluoro-benzothiophene-3- carbonitrile (Isomer 1) | 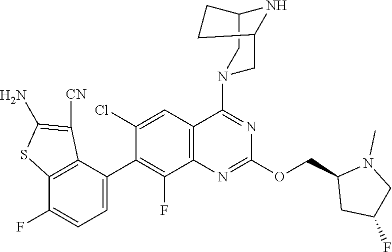 [SMILES: CN1C[C@H](F)C[C@H]1COc1nc(N2CC3CCC(C2)N3)c2cc(Cl)c(-c3ccc(F)c4sc(N)c(C#N)c34)c(F)c2n1] | 614 |
| 6 | 2-amino-4-[6-chloro-4-(3,8- diazabicyclo[3.2.1]octan-3- yl)-8-fluoro-2-[[(2R)- tetrahydrofuran-2- yl]methoxy]quinazolin-7-yl]- 7-fluoro-benzothiophene-3- carbonitrile (Isomer 2) | 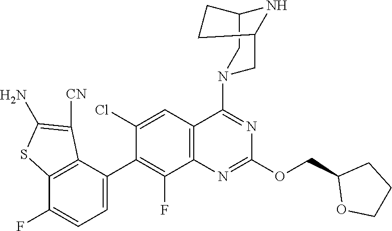 [SMILES: N#Cc1c(N)sc2c(F)ccc(-c3c(Cl)cc4c(N5CC6CCC(C5)N6)nc(OC[C@H]5CCCO5)nc4c3F)c12] | 583 |
| 7 | 2-amino-4-[6-chloro-4-(3,8- diazabicyclo[3.2.1]octan-3- yl)-8-fluoro-2-[[(2R,8S)-2- fluoro-1,2,3,5,6,7- hexahydropyrrolizin-8- yl]methoxy]quinazolin-7-yl]- 7-fluoro-benzothiophene-3- carbonitrile (Isomer 2) | 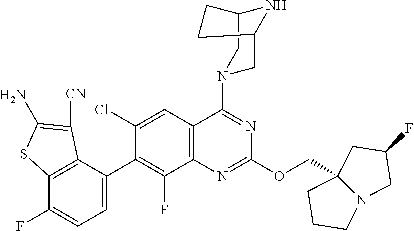 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12] | 640 |
| 8 | 2-amino-4-[6-chloro-4-(3,8- diazabicyclo[3.2.1]octan-3- yl)-8-fluoro-2-[[(2R,8S)-2- fluoro-1,2,3,5,6,7- hexahydropyrrolizin-8- yl]methoxy]quinazolin-7-yl]- 7-fluoro-benzothiophene-3- carbonitrile (Isomer 1) | 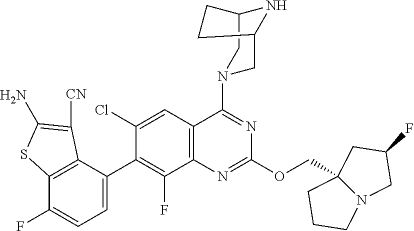 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12] | 640 |
| 9 | 2-amino-4-[6-chloro-8-fluoro- 2-[[(2S)-1-(2- fluoroethyl)pyrrolidin-2- yl]methoxy]-4-(3-oxa-7,9- diazabicyclo[3.3.1]nonan-9- yl)quinazolin-7-yl]-7-fluoro- benzothiophene-3-carbonitrile | 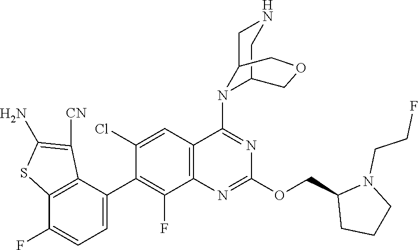 [SMILES: N#Cc1c(N)sc2c(F)ccc(-c3c(Cl)cc4c(N5C6CNCC5COC6)nc(OC[C@@H]5CCCN5CCF)nc4c3F)c12] | 644 |

### Preparation 39

### 2-amino-4-bromo-5-chloro-3-fluoro-benzoic acid

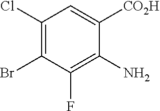 [SMILES: Nc1c(C(=O)O)cc(Cl)c(Br)c1F]

2-Amino-4-bromo-3-fluoro-benzoic acid (200.0 g, 854 mmol) was added to DMF (850 mL) followed by NCS (114.7 g, 854 mmol), which was added in four equal portions every 15 min. The mixture was stirred at rt for ˜18 h. Another portion of NCS (23.8 g, 179 mmol) was added and the reaction was stirred at rt for another 72 h. The mixture was poured into H₂O (4 L) and was stirred, filtered and dried under house vacuum at 50° C. to obtain the product (214 g, 93%) as a beige solid. MS (ES) m/z=267 (M-1).

### Preparation 40

### 7-bromo-6-chloro-8-fluoro-2-thioxo-1H-quinazolin-4-one

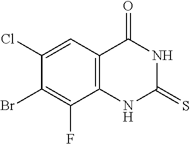 [SMILES: O=c1[nH]c(=S)[nH]c2c(F)c(Br)c(Cl)cc12]

2-Amino-4-bromo-5-chloro-3-fluoro-benzoic acid (213.7 g, 795.9 mmol) was added in portions to SOCl₂ (500 mL) over 5-10 min. The flask was then fitted with an HCl trap and was heated at reflux for 5 h. The homogeneous mixture was allowed to cool to rt and was stirred for 18 h. The mixture was concentrated, and DCM (˜500 mL) was added and removed under reduced pressure 2 times. A separate 5 L flask, fitted with an overhead stirrer and an internal thermometer, was charged with NH₄SCN (68 g, 871 mmol) and acetone (530 mL) and was placed under a blanket of N₂. A solution of the acid chloride in acetone (1060 mL) was added via an addition funnel over 1 h at a rate that maintained the internal temperature at or below 55° C. The reaction was stirred and allowed to cool with stirring for 18 h. The mixture was concentrated to ˜500 mL. The solid was collected by filtration and was slurried and rinsed with acetone 3 times and dried under vacuum for 18 h to afford the product (270 g, 90%) as a light-brown solid. MS (ES) m/z=307 (M-1).

### Preparation 41

### 7-bromo-6-chloro-8-fluoro-2-methylsulfanyl-1H-quinazolin-4-one

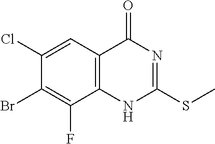 [SMILES: CSc1nc(=O)c2cc(Cl)c(Br)c(F)c2[nH]1]

To a flask containing 7-bromo-6-chloro-8-fluoro-2-thioxo-1H-quinazolin-4-one (106.6 g, 241 mmol) was added EtOH (1.2 L), NaOH (5 mol/L) in H₂O (51 mL, 255 mmol), followed by CH₃I (15.7 mL, 252 mmol) over 5 min. The reaction mixture was stirred at rt for 18 h. DCM (400 mL) and MeOH (200 mL) was introduced to help obtain a homogeneous solution. More NaOH (5 mol/L) in H₂O (14.5 mL, 72.5 mmol) and CH₃I (4.5 mL, 72 mmol) were added and the reaction was stirred at rt for 18 h. The mixture was poured into DCM (3 L), partitioned and the organic solution was concentrated to ˜100-200 mL. The solids were filtered and rinsed with DCM, H₂O, ACN, Et₂O and dried under vacuum at ˜50° C. to obtain the product (18 g, 24%) as a tan solid. MS (ES) m/z=321 (M-1).

### Preparation 42

### tert-Butyl-4-(7-bromo-6-chloro-8-fluoro-2-methylsulfanyl-quinazolin-4-yl)piperazine-1-carboxylate

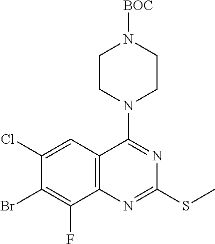 [SMILES: CSc1nc(N2CCN(C(=O)OC(C)(C)C)CC2)c2cc(Cl)c(Br)c(F)c2n1]

A mixture of 7-bromo-6-chloro-8-fluoro-2-methylsulfanyl-1H-quinazolin-4-one (18.2 g, 56.3 mmol) in DCM (110 mL), POCl₃ (13 mL, 138.1 mmol) and DIEA (10 mL, 57.3 mmol) was heated at 100° C. for 4 h. The heat was turned off and the reaction was left to stir at rt for 18 h. The mixture was concentrated in vacuo and azeotroped twice with DCM and dried under house vacuum to give a dark brown solid (38.6 g, 56.4 mmol) which was dissolved in 1,4-dioxane (110 mL) and DIEA (30 mL, 172 mmol) and was treated with tert-butyl piperazine-1-carboxylate (11 g, 57.88 mmol). The resulting mixture was stirred at rt under N₂ for 1.5 h. The reaction mixture was then partitioned between EtOAc and sat. aq. NaHCO₃ and sat. aq. NaCl. The layers were separated, and the aqueous layer was extracted 2× with EtOAc. The organic layers were combined and dried over anhydrous Na₂SO₄ and were concentrated to a brown oil. The oil was purified via silica gel chromatography, eluting with 100% hexanes to 20% EtOAc in hexanes to obtain the product (20.2 g, 73%) as a light-tan solid. MS (ES) m/z=491 (M+1).

### Preparation 43

### tert-Butyl 4-[7-[2-(tert-butoxycarbonylamino)-3-cyano-7-fluoro-benzothiophen-4-yl]-6-chloro-8-fluoro-2-methylsulfanyl-quinazolin-4-yl]piperazine-1-carboxylate

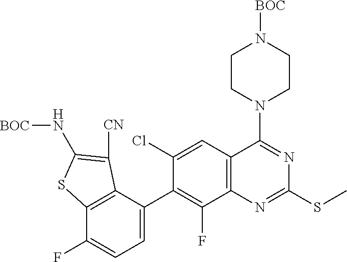 [SMILES: CSc1nc(N2CCN(C(=O)OC(C)(C)C)CC2)c2cc(Cl)c(-c3ccc(F)c4sc(NC(=O)OC(C)(C)C)c(C#N)c34)c(F)c2n1]

tert-Butyl 4-(7-bromo-6-chloro-8-fluoro-2-methylsulfanyl-quinazolin-4-yl)piperazine-1-carboxylate was prepared in the same manner as described in Preparation 36 to afford the product (6.8 g, 52%) as a pale yellow foam. MS (ES) m/z=703 (M+1).

### Preparation 44

### tert-Butyl 4-[7-[2-(tert-butoxycarbonylamino)-3-cyano-7-fluoro-benzothiophen-4-yl]-6-chloro-8-fluoro-2-methylsulfonyl-quinazolin-4-yl]piperazine-1-carboxylate

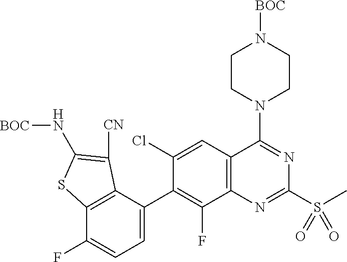 [SMILES: CC(C)(C)OC(=O)Nc1sc2c(F)ccc(-c3c(Cl)cc4c(N5CCN(C(=O)OC(C)(C)C)CC5)nc(S(C)(=O)=O)nc4c3F)c2c1C#N]

tert-Butyl 4-[7-[2-(tert-butoxycarbonylamino)-3-cyano-7-fluoro-benzothiophen-4-yl]-6-chloro-8-fluoro-2-methylsulfanyl-quinazolin-4-yl]piperazine-1-carboxylate was prepared in the same manner as described in Preparation 37 to afford the product (5.6 g, 79%) as a white powder. MS (ES) m/z=735 (M+1).

### Preparation 45

### [(2S)-1-allylpyrrolidin-2-yl] methanol

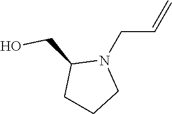

To a solution of L-prolinol (2.0 g, 19.2 mmol) in ACN (95 mL), was added K₂CO₃ (3.97 g, 28.8 mmol) and 3-bromoprop-1-ene (2.44 g, 20.1 mmol) at 0° C. The resulting mixture was stirred and slowly warmed to rt over 18 h under N₂. The reaction mixture was filtered through a diatomaceous earth and rinsed with EtOAc. The filtrate was concentrated, and the residue was purified via silica gel chromatography, eluting with 2% to 10% MeOH in DCM to obtain the product (1.9 g, 70%) as a light-yellow oil. MS (ES) m/z=142 (M+1).

### Preparation 46

### tert-Butyl 4-[2-[[(2S)-1-allylpyrrolidin-2-yl]methoxy]-7-[2-(tert-butoxycarbonylamino)-3-cyano-7-fluoro-benzothiophen-4-yl]-6-chloro-8-fluoro-quinazolin-4-yl]piperazine-1-carboxylate

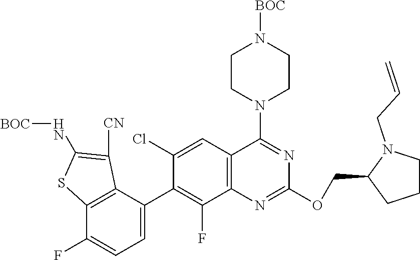 [SMILES: C=CCN1CCC[C@H]1COc1nc(N2CCN(C(=O)OC(C)(C)C)CC2)c2cc(Cl)c(-c3ccc(F)c4sc(NC(=O)OC(C)(C)C)c(C#N)c34)c(F)c2n1]

A 5 mL microwave tube was charged with pre-dried, powdered 4 Å molecular sieves (0.4 g) and Cs₂CO₃ (0.443 g, 1.36 mmol) and was dried at 100° C. for 2 h. To the pre-dried tube was added a solution of tert-butyl 4-[7-[2-(tert-butoxycarbonylamino)-3-cyano-7-fluoro-benzothiophen-4-yl]-6-chloro-8-fluoro-2-methylsulfonyl-quinazolin-4-yl]piperazine-1-carboxylate (0.20 g, 0.27 mmol) and [(2S)-1-allylpyrrolidin-2-yl]methanol (0.154 g, 1.09 mmol) in DMSO (1.0 mL). The resulting mixture was stirred at 100° C. for 1 h. The mixture was diluted with EtOAc and was filtered to remove solids. The organics were washed with H₂O, sat. aq. NaCl, dried over anhydrous Na₂SO₄, filtered and concentrated. The residue was purified via silica gel chromatography, eluting with a gradient of 20% to 30% acetone in hexanes to obtain the product (0.18 g, 83%). MS (ES) m/z=796 (M+1).

### Preparation 47

### 4-[2-[[(2S)-1-Allylpyrrolidin-2-yl]methoxy]-6-chloro-8-fluoro-4-piperazin-1-yl-quinazolin-7-yl]-2-amino-7-fluoro-benzothiophene-3-carbonitrile

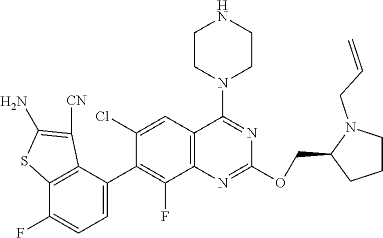 [SMILES: C=CCN1CCC[C@H]1COc1nc(N2CCNCC2)c2cc(Cl)c(-c3ccc(F)c4sc(N)c(C#N)c34)c(F)c2n1]

tert-Butyl 4-[2-[[(2S)-1-allylpyrrolidin-2-yl]methoxy]-7-[2-(tert-butoxycarbonylamino)-3-cyano-7-fluoro-benzothiophen-4-yl]-6-chloro-8-fluoro-quinazolin-4-yl]piperazine-1-carboxylate was prepared in the same manner as described in Example 1 to afford the product (0.078 g, 99%). MS (ES) m/z=596 (M+1).

### Example 10

### 2-amino-4-[6-chloro-8-fluoro-4-piperazin-1-yl-2-[[(2S)-1-propylpyrrolidin-2-yl]methoxy]quinazolin-7-yl]-7-fluoro-benzothiophene-3-carbonitrile

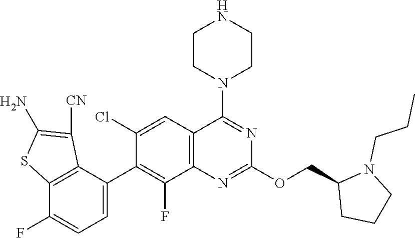 [SMILES: CCCN1CCC[C@H]1COc1nc(N2CCNCC2)c2cc(Cl)c(-c3ccc(F)c4sc(N)c(C#N)c34)c(F)c2n1]

A vial containing 4-[2-[[(2S)-1-allylpyrrolidin-2-yl]methoxy]-6-chloro-8-fluoro-4-piperazin-1-yl-quinazolin-7-yl]-2-amino-7-fluoro-benzothiophene-3-carbonitrile (38.4 mg, 0.0644 mmol) was placed into a glove box and a stir bar was added. (dippf)Rh(cod)BF₄ (11 mg, 0.015 mmol) and MeOH (3 mL) were added. The vial was capped and removed from the glove box. The vial was placed into an autoclave. A needle was inserted through the cap to allow gas flow. The autoclave was sealed and purged 3× with H₂. The reaction mixture was brought to a final pressure of 150 psi of H₂. After 18 h, the reaction was vented, and the mixture concentrated. The residue was dissolved in DCM and purified via silica gel chromatography, eluting with a gradient of 100% EtOAc to 100% (5% Et₃N/ACN), then with 100% MeOH to obtain the product (0.015 g, 31%). MS (ES) m/z=598 (M+1).

### Preparation 48

### 2-Amino-4-bromo-5-chloro-3-fluoro-benzoic acid

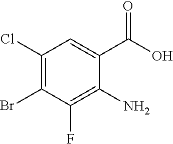 [SMILES: Nc1c(C(=O)O)cc(Cl)c(Br)c1F]

2-Amino-4-bromo-3-fluoro-benzoic acid (200 g, 854 mmol) and DMF (850 mL) were combined in a 2 L round bottom flask and charged with NCS (136.3 g, 1.02 mol, 1.2 eq.) in two roughly equal portions, one hour apart and was stirred at rt for 18 h. The reaction mixture was poured into H₂O (4 L), stirred with a spatula and the product was collected by filtration, rinsed with H₂O and dried in a vacuum oven at 50-60° C. to obtain the product (216.3 g, 94%) as a beige solid. MS (ES) m/z=266/268 (M-1).

### Preparation 49

### 7-Bromo-6-chloro-8-fluoro-1H-quinazoline-2,4-dione

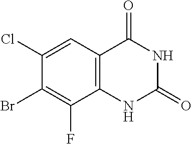 [SMILES: O=c1[nH]c(=O)c2cc(Cl)c(Br)c(F)c2[nH]1]

In a 1 L round bottom flask fitted with a condenser, a mixture of 2-amino-4-bromo-5-chloro-3-fluoro-benzoic acid (50.0 g, 186.2 mmol) and urea (56.0 g, 932 mmol, 5 eq.) was heated at 200° C. for 4 h. The reaction was allowed to cool to rt at which point a brown solid formed in the flask. The material was scraped loose from the flask and the large pieces were ground with a mortar and pestle to a brown solid. EtOAc and H₂O were added, and the mixture was stirred vigorously at 70° C. for 2 h. The mixture was filtered and rinsed with additional EtOAc, to give a light-brown solid. The wet solid was dried under house vacuum overnight to obtain the product (55.3 g, quantitative) as a light-brown solid. MS (ES) m/z=291/293 (M-1).

### Preparation 50

### 7-Bromo-2,4,6-trichloro-8-fluoro-quinazoline

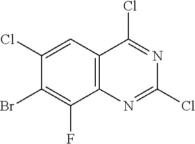 [SMILES: Fc1c(Br)c(Cl)cc2c(Cl)nc(Cl)nc12]

In a 500 mL round bottom flask fitted with a condenser, a mixture of 7-bromo-6-chloro-8-fluoro-1H-quinazoline-2,4-dione (23.9 g, 81.4 mmol), POCl₃ (130 mL, 1381 mmol) and DIPEA (35 mL, 201 mmol, 2.5 eq.) was heated at 105° C. for ˜72 h. The mixture was concentrated in vacuo and was azeotroped 2× with toluene to give a dark brown oil. The oil was purified by silica gel chromatography, eluting with 5% to 20% acetone in hexanes to obtain the product (14.6 g, 54%) as a pale orange solid. MS (ES) m/z=329/331/333 (M+1).

### Preparation 51

### tert-Butyl 4-(7-bromo-2,6-dichloro-8-fluoro-quinazolin-4-yl)piperazine-1-carboxylate

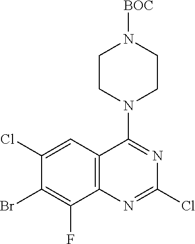 [SMILES: CC(C)(C)OC(=O)N1CCN(c2nc(Cl)nc3c(F)c(Br)c(Cl)cc23)CC1]

tert-Butyl piperazine-1-carboxylate (7.0 g, 37 mmol, 1 eq) and DIEA (19 mL, 109 mmol, 3 eq.) were added to a stirred mixture of 7-bromo-2,4,6-trichloro-8-fluoro-quinazoline (12.0 g, 36.3 mmol) in 1,4-dioxane (145 mL) and was heated to 50° C. for 1 h. The reaction was cooled to rt, diluted with EtOAc and was washed with H₂O and sat. aq. NaCl, dried over anhydrous Na₂SO₄, filtered and concentrated. The residue was purified via silica gel chromatography, eluting with 100% hexanes to 30% EtOAc in hexanes to obtain the product (12.4 g, 71%) as an off-white solid. MS (ES) m/z=471/481/483 (M+1).

### Preparation 52

### tert-Butyl 4-[7-bromo-6-chloro-8-fluoro-2-[[(2S)-1-methylpyrrolidin-2-yl]methoxy]quinazolin-4-yl]piperazine-1-carboxylate

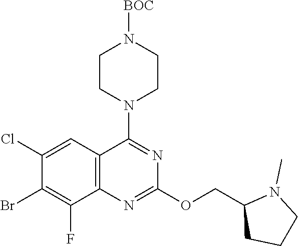 [SMILES: CN1CCC[C@H]1COc1nc(N2CCN(C(=O)OC(C)(C)C)CC2)c2cc(Cl)c(Br)c(F)c2n1]

tert-Butyl 4-(7-bromo-2,6-dichloro-8-fluoro-quinazolin-4-yl)piperazine-1-carboxylate (3.0 g, 6.2 mmol) was combined with N-methyl-L-prolinol (2.9 g, 25 mmol, 4 eq.) and KF (2.2 g, 38 mmol, 6 eq) in DMSO (20 mL, 280 mmol, 100 mass %) in a microwave and was heated in a microwave at 90° C. for 2 h. The reaction solution was diluted with EtOAc and was washed with H₂O and sat. aq. NaCl. The organic layer was dried over anhydrous Na₂SO₄, filtered and was concentrated. The residue was purified via silica gel chromatography, eluting with 100% DCM to 10% MeOH in DCM to give the product (1.4 g, 40%) as a tan foam. MS (ES) m/z=558 (M+1).

### Preparation 53

### tert-Butyl 4-[7-[2-(tert-butoxycarbonylamino)-3-cyano-7-fluoro-benzothiophen-4-yl]-6-chloro-8-fluoro-2-[[(2S)-1-methylpyrrolidin-2-yl]methoxy]quinazolin-4-yl]piperazine-1-carboxylate

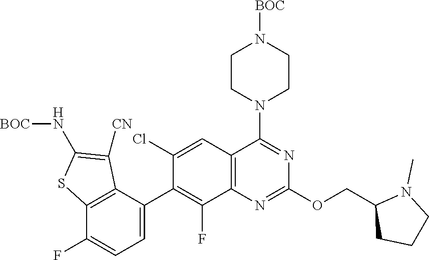 [SMILES: CN1CCC[C@H]1COc1nc(N2CCN(C(=O)OC(C)(C)C)CC2)c2cc(Cl)c(-c3ccc(F)c4sc(NC(=O)OC(C)(C)C)c(C#N)c34)c(F)c2n1]

To a vial containing tert-butyl 4-[7-bromo-6-chloro-8-fluoro-2-[[(2S)-1-methylpyrrolidin-2-yl]methoxy]quinazolin-4-yl]piperazine-1-carboxylate (0.376 g, 0.672 mmol), tert-butyl N-[3-cyano-4-(5,5-dimethyl-1,3,2-dioxaborinan-2-yl)-7-fluoro-benzothiophen-2-yl]carbamate (0.353 g, 0.873 mmol, 2.5 eq), DPEPhosPdCl₂ (0.096 g, 0.134 mmol, 0.2 eq) and Cs₂CO₃ (0.657 g, 2.02 mmol, 3 eq) was added toluene (8.4 mL). The flask was evacuated and refilled with N₂ (3×), then was placed in a heating block set at 125° C. for 1.5 h. The mixture was filtered through diatomaceous earth, rinsing with DCM and ˜20 mL 9:1 DCM/MeOH. The filtrate was concentrated and purified via silica gel chromatography, eluting with 100% DCM to 20% MeOH to obtain the product (0.224 g, 43%). MS (ES) m/z=770 (M+1).

### Preparation 54

### 2-Amino-4-[6-chloro-8-fluoro-2-[[(2S)-1-methylpyrrolidin-2-yl]methoxy]-4-piperazin-1-yl-quinazolin-7-yl]-7-fluoro-benzothiophene-3-carbonitrile

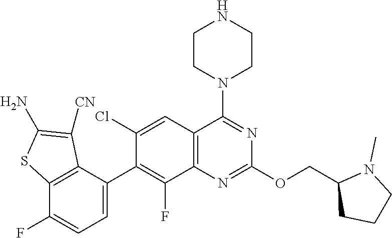 [SMILES: CN1CCC[C@H]1COc1nc(N2CCNCC2)c2cc(Cl)c(-c3ccc(F)c4sc(N)c(C#N)c34)c(F)c2n1]

To a solution of tert-butyl 4-[7-[2-(tert-butoxycarbonylamino)-3-cyano-7-fluoro-benzothiophen-4-yl]-6-chloro-8-fluoro-2-[[(2S)-1-methylpyrrolidin-2-yl]methoxy]quinazolin-4-yl]piperazine-1-carboxylate (0.098 g, 0.13 mmol) in DCM (2 mL) was added TFA (1 mL) and was stirred at rt for 2 h. The reaction was concentrated and was filtered through a 10 g SCX column eluting with MeOH followed by 7N ammoniated MeOH. The filtrate was concentrated, and the residue purified via silica gel chromatography, eluting with 4% to 20% 7N NH₃ in MeOH in DCM to obtain the product (0.045 g, 52%). MS (ES) m/z=570 (M+1).

### Preparation 55

### Chiral Purification

### tert-Butyl 4-[7-[2-(tert-butoxycarbonylamino)-3-cyano-7-fluoro-benzothiophen-4-yl]-6-chloro-8-fluoro-2-[[(2S)-1-methylpyrrolidin-2-yl]methoxy]quinazolin-4-yl]piperazine-1-carboxylate, Isomer 2

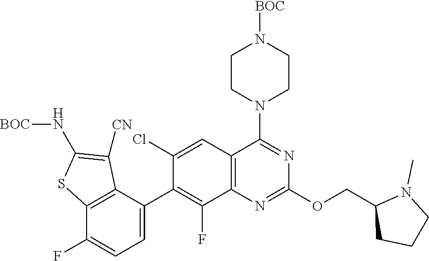 [SMILES: CN1CCC[C@H]1COc1nc(N2CCN(C(=O)OC(C)(C)C)CC2)c2cc(Cl)c(-c3ccc(F)c4sc(NC(=O)OC(C)(C)C)c(C#N)c34)c(F)c2n1]

tert-Butyl 4-[7-[2-(tert-butoxycarbonylamino)-3-cyano-7-fluoro-benzothiophen-4-yl]-6-chloro-8-fluoro-2-[[(2S)-1-methylpyrrolidin-2-yl]methoxy]quinazolin-4-yl]piperazine-1-carboxylate (1.0 g, 175 mmol) was dissolved in MeOH (19 mL) and was purified via SFC using a CHIRALPAK® AD-H (5×15 cm), 60/40 CO₂/IPA w/0.5% DMEA (Flow=300 g/min, Pressure=184 bar, 295 nm) to afford the separate the compound as Isomer 1 (0.321 g, 56 mmol, ee>99%) and Isomer 2 (0.505 g, 88 mmol, ee>99%).

### Example 11

### 2-Amino-4-[6-chloro-8-fluoro-2-[[(2S)-1-methylpyrrolidin-2-yl]methoxy]-4-piperazin-1-yl-quinazolin-7-yl]-7-fluoro-benzothiophene-3-carbonitrile, Isomer 2

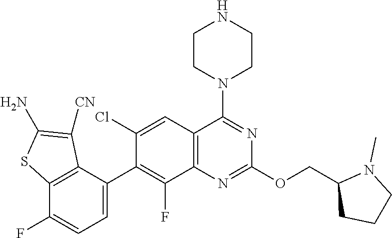 [SMILES: CN1CCC[C@H]1COc1nc(N2CCNCC2)c2cc(Cl)c(-c3ccc(F)c4sc(N)c(C#N)c34)c(F)c2n1]

tert-Butyl 4-[7-[2-(tert-butoxycarbonylamino)-3-cyano-7-fluoro-benzothiophen-4-yl]-6-chloro-8-fluoro-2-[[(2S)-1-methylpyrrolidin-2-yl]methoxy]quinazolin-4-yl]piperazine-1-carboxylate (0.505 g, 0.66 mmol) was stirred in DCM (5 mL) and was cooled to 0° C. TFA (2.5 mL) was added, and the reaction was stirred for 2 h. The reaction solution was filtered through an SCX column (10 g), washed with 3 column volumes of MeOH then was eluted off the column with 3 column volumes of 2N NH₃ in MeOH. The basic filtrate was concentrated to obtain the product (0.251 g, 67%) as a tan solid. MS (ES) m/z=570 (M+1).

### Example 12

### 2-Amino-4-[6-chloro-8-fluoro-2-[[(2S)-1-methylpyrrolidin-2-yl]methoxy]-4-[4-(2,2,2-trifluoroacetyl)piperazin-1-yl]quinazolin-7-yl]-7-fluoro-benzothiophene-3-carbonitrile

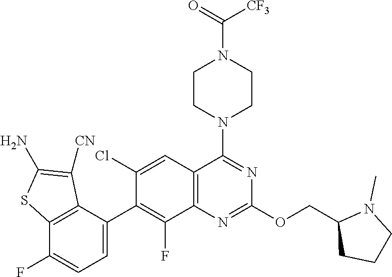 [SMILES: CN1CCC[C@H]1COc1nc(N2CCN(C(=O)C(F)(F)F)CC2)c2cc(Cl)c(-c3ccc(F)c4sc(N)c(C#N)c34)c(F)c2n1]

To a vial containing 2-amino-4-[6-chloro-8-fluoro-2-[[(2S)-1-methylpyrrolidin-2-yl]methoxy]-4-piperazin-1-yl-quinazolin-7-yl]-7-fluoro-benzothiophene-3-carbonitrile (0.100 g, 0.17 mmol), HATU (0.204 g, 0.53 mmol) and DMF (2 mL) was added TFA (0.04 mL, 0.6 mmol) and DIEA (0.12 mL, 0.6 mmol). The reaction was stirred at rt for ˜18 h. The reaction solution was diluted with EtOAc and washed with H₂O and sat. aq. NaCl. The organic layer was dried over anhydrous Na₂SO₄, filtered and concentrated. The residue was purified via reversed phase chromatography (HPH Phenomenex Kinetix EVO 2.6u, 2.1×50 mm, 1.1 mL/min, 1.8 min. gradient 5-100% B, 2 min run time, Solvent A: 10 mM ammonium bicarbonate. Solvent B: ACN) to obtain the product (0.038 g, 33%) as a white solid. MS (ES) m/z=666 (M+1).

### Preparation 56

### 7-Bromo-6-chloro-2-ethylsulfanyl-8-fluoro-quinazoline

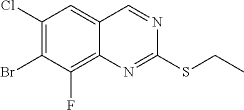 [SMILES: CCSc1ncc2cc(Cl)c(Br)c(F)c2n1]

A solution of 7-bromo-4,6-dichloro-2-ethylsulfanyl-8-fluoro-quinazoline (19 g, 53.37 mmol) in THE (266 mL) was sparged with N₂ for 2 min. 1,1′-bis(diphenylphosphino)ferrocene-palladium(II)dichloride dichloromethane complex (2.2 g, 2.6 mmol), N,N,N′,N′-tetramethylethylenediamine (9.6 mL, 64 mmol) and NaBH₃CN (4.02 g, 64.0 mmol) were added and were stirred at rt for 35 minutes. Sat. aq. NH₄Cl (300 mL) was added, and the mixture extracted with EtOAc (3×200 mL). The organics were washed with sat. aq. NaCl, dried over anhydrous Na₂SO₄ and concentrated. The residue (35 g) was dissolved in DCM (60 mL) and filtered through a plug of silica (200 g), eluting with DCM to obtain the product (16.2 g, 92%) as a light-yellow solid. MS (ES+) m/z=321 (M+1).

### Preparation 57

### tert-Butyl N-[4-(6-chloro-2-ethylsulfanyl-8-fluoro-quinazolin-7-yl)-3-cyano-7-fluoro-benzothiophen-2-yl]carbamate

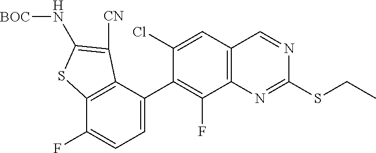 [SMILES: CCSc1ncc2cc(Cl)c(-c3ccc(F)c4sc(NC(=O)OC(C)(C)C)c(C#N)c34)c(F)c2n1]

A 1 L flask was charged with toluene (300 mL) and was sparged with N₂ for 60 min. at 50° C. Then 7-bromo-6-chloro-2-ethylsulfanyl-8-fluoro-quinazoline (7.00 g, 21.8 mmol), tert-butyl N-[3-cyano-4-(5,5-dimethyl-1,3,2-dioxaborinan-2-yl)-7-fluoro-benzothiophen-2-yl]carbamate (11.4 g, 28.2 mmol) and Cs₂CO₃ (21.3 g, 65.4 mmol) were added, followed by DPEPhosPdCl₂ (3.11 g, 4.35 mmol). The mixture was then heated at 120° C. for 1.5 h. The mixture was filtered through diatomaceous earth and was rinsed with 1:1 EtOAc/MTBE (500 mL). The filtrate was washed with NaHCO₃, H₂O, sat. aq. NaCl, dried over anhydrous MgSO₄, filtered, and concentrated. The residue was purified via silica gel chromatography, eluting with a gradient of 0 to 80% EtOAc/hexanes to obtain the product (7.00 g, 60%) as light-yellow solid. MS (ES+) m/z=533 (M+1).

### Preparation 58

### tert-Butyl N-[4-(6-chloro-2-ethylsulfonyl-8-fluoro-quinazolin-7-yl)-3-cyano-7-fluoro-benzothiophen-2-yl]carbamate

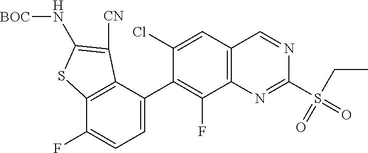 [SMILES: CCS(=O)(=O)c1ncc2cc(Cl)c(-c3ccc(F)c4sc(NC(=O)OC(C)(C)C)c(C#N)c34)c(F)c2n1]

tert-Butyl N-[4-(6-chloro-2-ethylsulfanyl-8-fluoro-quinazolin-7-yl)-3-cyano-7-fluoro-benzothiophen-2-yl]carbamate (6.95 g, 13 mmol) and DCM (200 mL) were combined in a round bottom flask under nitrogen. mCPBA (8.40 g, 34.1 mmol) was added in one portion and was stirred at rt for 1 h. 10% aqueous Na₂S₂O₃ was added, and the organic phase was washed with NaHCO₃ solution. The isolated organic layer was dried over anhydrous Na₂SO₄, filtered, and concentrated. The residue was purified via silica gel chromatography, eluting with 0 to 80% EtOAc/Hexane to obtain the product (5.50 g, 75%) as a white solid. MS (ES+) m/z=565 (M+1).

### Preparation 59

### tert-Butyl N-[4-[6-chloro-8-fluoro-2-[[(2S)-1-methylpyrrolidin-2-yl]methoxy]quinazolin-7-yl]-3-cyano-7-fluoro-benzothiophen-2-yl]carbamate

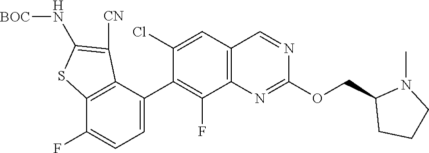 [SMILES: CN1CCC[C@H]1COc1ncc2cc(Cl)c(-c3ccc(F)c4sc(NC(=O)OC(C)(C)C)c(C#N)c34)c(F)c2n1]

A solution of N-methyl-L-prolinol (0.05 g, 0.4 mmol) in THE (1.5 mL) was charged with lithium bis(trimethylsilyl)amide (1M in THE (0.6 mL, 0.6 mmol)) in one portion and was stirred at rt for 5 min. Solid tert-butyl N-[4-(6-chloro-2-ethylsulfonyl-8-fluoro-quinazolin-7-yl)-3-cyano-7-fluoro-benzothiophen-2-yl]carbamate (0.150 g, 0.265 mmol) was added in one portion and was stirred at rt for 0.5 h. The mixture was diluted with EtOAc and was washed with sat. aq. NH₄Cl and sat. aq. NaCl. The organics were dried over anhydrous Na₂SO₄, filtered and were concentrated. The residue was purified via silica gel chromatography, eluting with a gradient of 0-10% MeOH in DCM to obtain the product (0.086 g, 55%) as a yellow solid. MS (ES+) m/z=586 (M+1).

### Example 13

### 2-Amino-4-[6-chloro-8-fluoro-2-[[(2S)-1-methylpyrrolidin-2-yl]methoxy]quinazolin-7-yl]-7-fluoro-benzothiophene-3-carbonitrile

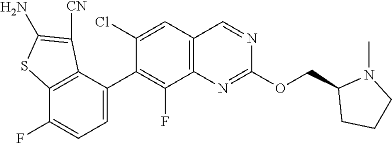 [SMILES: CN1CCC[C@H]1COc1ncc2cc(Cl)c(-c3ccc(F)c4sc(N)c(C#N)c34)c(F)c2n1]

A solution of 2-amino-4-[6-chloro-8-fluoro-2-[[(2S)-1-methylpyrrolidin-2-yl]methoxy]quinazolin-7-yl]-7-fluoro-benzothiophene-3-carbonitrile (0.100 g, 0.171 mmol) in DCM (0.5 mL) was charged with TFA (0.7 mL) and was stirred at rt for ˜18 h. The mixture was concentrated, DCM added, and concentrated once more. The residue was purified via silica gel chromatography, eluting with 10% 7N NH₃ in MeOH in DCM to obtain the product (0.022 g, 27%) as a yellow solid. MS (ES+) m/z=486 (M+1).

### Examples 14 and 15

### 2-Amino-4-[6-chloro-8-fluoro-2-[[(2S)-1-methylpyrrolidin-2-yl]methoxy]quinazolin-7-yl]-7-fluoro-benzothiophene-3-carbonitrile

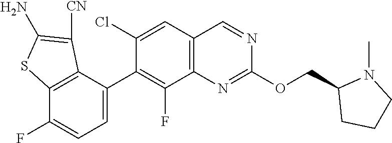 [SMILES: CN1CCC[C@H]1COc1ncc2cc(Cl)c(-c3ccc(F)c4sc(N)c(C#N)c34)c(F)c2n1]

The atropisomers of 2-Amino-4-[6-chloro-8-fluoro-2-[[(2S)-1-methylpyrrolidin-2-yl]methoxy]quinazolin-7-yl]-7-fluoro-benzothiophene-3-carbonitrile (0.057 g, 0.12 mmol) were separated via SFC (CHIRALPAK® AD-H, 4.6×150 mm, 40% MeOH (0.2% IPAm)/CO₂, 5 mL/min, 225 nm) to obtain the products (Isomer 1, 0.012 g, ee>99%; Isomer 2, 0.010 g, ee>99%) as white solids. Isomer 1: MS (ES+) m/z=486 (M+1); Isomer 2: MS (ES+) m/z=485.8 (M+1).

The Example compounds in Table 4 were prepared in a similar manner as described in Preparation 59 and deprotected in a similar manner to Example 13. Various methods were used to purify the compounds, which would be apparent to one skilled in the art.

TABLE 4
Example Compounds 16 to 30.

| | | | MS |
|---|---|---|---|
| | | | (ES) m/z |
| Example | Chemical Name | Structure | (M + H) |
| 16 | 2-amino-4-[6-chloro-8-fluoro-2- [(1-methylazetidin-2- yl)methoxy]quinazolin-7-yl]-7- fluoro-benzothiophene-3- carbonitrile | 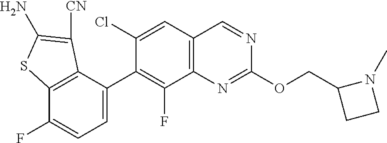 [SMILES: CN1CCC1COc1ncc2cc(Cl)c(-c3ccc(F)c4sc(N)c(C#N)c34)c(F)c2n1] | 472 |
| 17 | 2-amino-4-[6-chloro-8-fluoro-2- (1,2,3,5,6,7-hexahydropyrrolizin- 8-ylmethoxy)quinazolin-7-yl]-7- fluoro-benzothiophene-3- carbonitrile (isomer 2) | 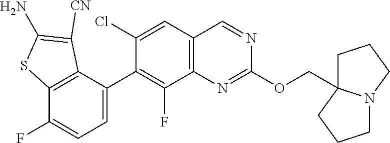 [SMILES: N#Cc1c(N)sc2c(F)ccc(-c3c(Cl)cc4cnc(OCC56CCCN5CCC6)nc4c3F)c12] | 512 |
| 18 | 2-amino-4-[6-chloro-8-fluoro-2- [[(2R)-1-methylpyrrolidin-2- yl]methoxy]quinazolin-7-yl]-7- fluoro-benzothiophene-3- carbonitrile | 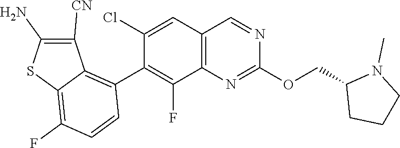 [SMILES: CN1CCC[C@@H]1COc1ncc2cc(Cl)c(-c3ccc(F)c4sc(N)c(C#N)c34)c(F)c2n1] | 486 |
| 19 | 2-amino-4-[6-chloro-8-fluoro-2- [1-[(2S)-1-methylpyrrolidin-2- yl]ethoxy]quinazolin-7-yl]-7- fluoro-benzothiophene-3- carbonitrile (isomer 1) | 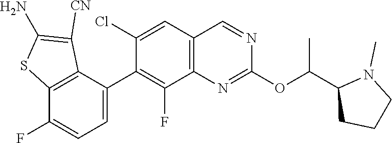 [SMILES: CC(Oc1ncc2cc(Cl)c(-c3ccc(F)c4sc(N)c(C#N)c34)c(F)c2n1)[C@@H]1CCCN1C] | 500 |
| 20 | 2-amino-4-[6-chloro-8-fluoro-2- [[(2S)-1-(2- hydroxyethyl)pyrrolidin-2- yl]methoxy]quinazolin-7-yl]-7- fluoro-benzothiophene-3- carbonitrile | 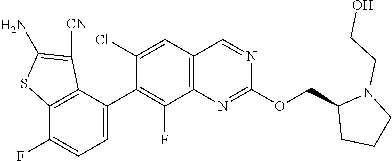 [SMILES: N#Cc1c(N)sc2c(F)ccc(-c3c(Cl)cc4cnc(OC[C@@H]5CCCN5CCO)nc4c3F)c12] | 516 |
| 21 | 2-amino-4-[6-chloro-2-[[(2S,4S)- 1,4-dimethylpyrrolidin-2- yl]methoxy]-8-fluoro-quinazolin- 7-yl]-7-fluoro-benzothiophene-3- carbonitrile (isomer 1) | 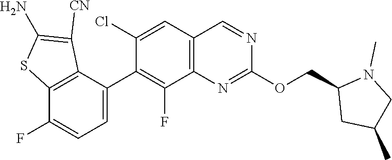 [SMILES: C[C@H]1C[C@@H](COc2ncc3cc(Cl)c(-c4ccc(F)c5sc(N)c(C#N)c45)c(F)c3n2)N(C)C1] | 500 |
| 22 | 2-amino-4-[6-chloro-2-[[(2S,4R)- 1,4-dimethylpyrrolidin-2- yl]methoxy]-8-fluoro-quinazolin- 7-yl]-7-fluoro-benzothiophene-3- carbonitrile (isomer 2) | 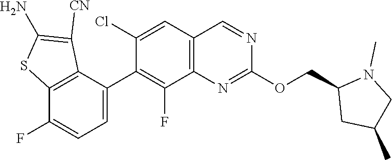 [SMILES: C[C@H]1C[C@@H](COc2ncc3cc(Cl)c(-c4ccc(F)c5sc(N)c(C#N)c45)c(F)c3n2)N(C)C1] | 500 |
| 23 | 2-amino-4-[6-chloro-8-fluoro-2- [[(1R,2S,5S)-3-methyl-3- azabicyclo[3.1.0]hexan-2- yl]methoxy]quinazolin-7-yl]-7- fluoro-benzothiophene-3- carbonitrile (isomer 1) | 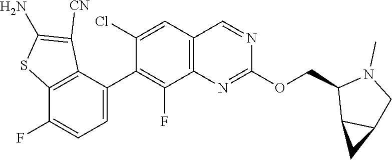 [SMILES: CN1C[C@H]2C[C@H]2[C@H]1COc1ncc2cc(Cl)c(-c3ccc(F)c4sc(N)c(C#N)c34)c(F)c2n1] | 498 |
| 24 | 2-amino-4-[6-chloro-8-fluoro-2- [[(1S,2S,5R)-3-methyl-3- azabicyclo[3.1.0]hexan-2- yl]methoxy]quinazolin-7-yl]-7- fluoro-benzothiophene-3- carbonitrile (isomer 2) | 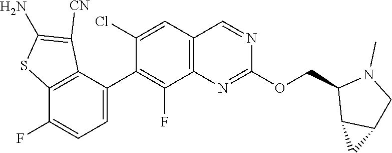 [SMILES: CN1C[C@@H]2C[C@@H]2[C@H]1COc1ncc2cc(Cl)c(-c3ccc(F)c4sc(N)c(C#N)c34)c(F)c2n1] | 498 |
| 25 | 2-amino-4-[6-chloro-8-fluoro-2- [[(2S,4R)-4-fluoro-1-methyl- pyrrolidin-2- yl]methoxy]quinazolin-7-yl]-7- fluoro-benzothiophene-3- carbonitrile (Isomer 1) | 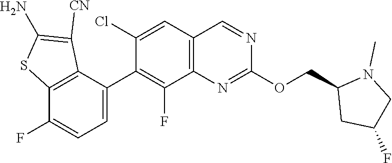 [SMILES: CN1C[C@H](F)C[C@H]1COc1ncc2cc(Cl)c(-c3ccc(F)c4sc(N)c(C#N)c34)c(F)c2n1] | 504 |
| 26 | 2-amino-4-[6-chloro-8-fluoro-2- [[2-(hydroxymethyl)-1-methyl- pyrrolidin-2- yl]methoxy]quinazolin-7-yl]-7- fluoro-benzothiophene-3- carbonitrile (Isomer 1) | 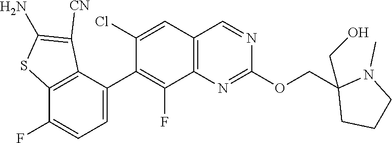 [SMILES: CN1CCCC1(CO)COc1ncc2cc(Cl)c(-c3ccc(F)c4sc(N)c(C#N)c34)c(F)c2n1] | 516 |
| 27 | 2-amino-4-[6-chloro-8-fluoro-2- [[2-(hydroxymethyl)-1-methyl- pyrrolidin-2- yl]methoxy]quinazolin-7-yl]-7- fluoro-benzothiophene-3- carbonitrile (Isomer 2) | 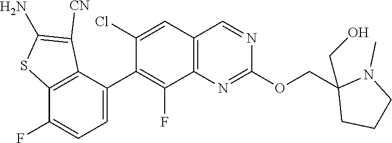 [SMILES: CN1CCCC1(CO)COc1ncc2cc(Cl)c(-c3ccc(F)c4sc(N)c(C#N)c34)c(F)c2n1] | 516 |
| 28 | 2-amino-4-[6-chloro-2-[[(2S)-1,2- dimethylpyrrolidin-2- yl]methoxy]-8-fluoro-quinazolin- 7-yl]-7-fluoro-benzothiophene-3- carbonitrile (Isomer 2) | 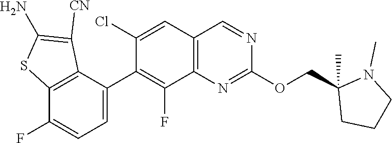 [SMILES: CN1CCC[C@@]1(C)COc1ncc2cc(Cl)c(-c3ccc(F)c4sc(N)c(C#N)c34)c(F)c2n1] | 499 |
| 29 | 2-amino-4-[6-chloro-8-fluoro-2- [[(2R,3S)-3-hydroxy-1-methyl- pyrrolidin-2- yl]methoxy]quinazolin-7-yl]-7- fluoro-benzothiophene-3- carbonitrile | 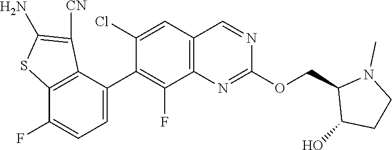 [SMILES: CN1CC[C@H](O)[C@H]1COc1ncc2cc(Cl)c(-c3ccc(F)c4sc(N)c(C#N)c34)c(F)c2n1] | 502 |
| 30 | 2-amino-4-[6-chloro-8-fluoro-2- [(1-[(2S)-1-methylpyrrolidin-2- yl]ethoxy]quinazolin-7-yl]-7- fluoro-benzothiophene-3- carbonitrile | 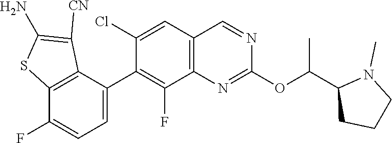 [SMILES: CC(Oc1ncc2cc(Cl)c(-c3ccc(F)c4sc(N)c(C#N)c34)c(F)c2n1)[C@@H]1CCCN1C] | 500 |

Example 19 was prepared from the alcohol in Preparation 6. Example 30 was prepared from the alcohol in Preparation 7.

### Preparation 60

### 7-Bromo-6-chloro-2-ethylsulfonyl-8-fluoro-quinazoline

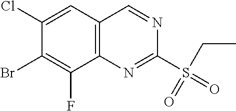 [SMILES: CCS(=O)(=O)c1ncc2cc(Cl)c(Br)c(F)c2n1]

mCPBA (6.55 g, 38.1 mmol) was added to a solution of 7-bromo-6-chloro-2-ethylsulfanyl-8-fluoro-quinazoline (4.10 g, 12.7 mmol) in DCM (65.0 mL) at 0° C. The ice bath was removed after 0.5 h and the reaction was stirred at rt for ˜18 h. The reaction was diluted with DCM and sat. aq. NaHCO₃, partitioned and the aqueous layer was extracted with EtOAc. The combined organic phases were washed with sat. aq. Na₂SO₄ solution, sat. aq. NaCl, dried over anhydrous MgSO₄, filtered and concentrated. The residue was purified via silica gel chromatography, eluting with 100% DCM then a gradient of 0-10% EtOAc in DCM to obtain the product (1.87 g. 42%) as a white solid. MS (ES+) m/z=353 (M+1).

### Preparation 61

### 7-Bromo-6-chloro-8-fluoro-2-[[(2R)-1-methylpyrrolidin-2-yl]methoxy]quinazoline

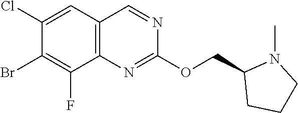 [SMILES: CN1CCC[C@H]1COc1ncc2cc(Cl)c(Br)c(F)c2n1]

Prepared from 7-bromo-6-chloro-2-ethylsulfonyl-8-fluoro-quinazoline in the same manner as described in Preparation 59 to afford the crude product (1.0 g. 46%) as a brown solid. MS (ES+) m/z=374 (M+1).

### Example 31

2-Amino-4-[6-chloro-8-fluoro-2-[[(2R)-1-methylpyrrolidin-2-yl]methoxy]quinazolin-7-yl]-7-methyl-benzothiophene-3-carbonitrile

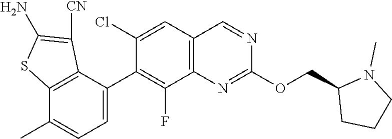 [SMILES: Cc1ccc(-c2c(Cl)cc3cnc(OC[C@@H]4CCCN4C)nc3c2F)c2c(C#N)c(N)sc12]

To a microwave vial was added tert-butyl N-[3-cyano-4-(5,5-dimethyl-1,3,2-dioxaborinan-2-yl)-7-methyl-benzothiophen-2-yl]carbamate (0.310 g, 0.774 mmol), (2R)-2-[(7-bromo-6-chloro-8-fluoro-quinazolin-2-yl)oxymethyl]pyrrolidin-1-amine (0.25 g, 0.67 mmol), Cs₂CO₃ (0.60 g, 1.8 mmol), and toluene (5.00 mL). The mixture was degassed for 3-4 minutes by passing a stream of N₂ through the mixture. Then dichloro[bis(2-(diphenylphosphino)phenyl)ether]palladium (II) (DPEPhosPdCl₂) (0.135 g, 0.185 mmol) was added. The vial was capped and was heated to 120° C. for 3 h. The mixture was diluted with EtOAc and filtered through a pad of diatomaceous earth. The filtrate was washed with H₂O, sat. aq. NaCl, dried over anhydrous Na₂SO₄, filtered and concentrated. The crude residue (0.130 g, 0.223 mmol) was dissolved in DCM (2.20 mL) and was treated with TFA (0.160 mL). The reaction was stirred at rt for ˜18 h. The mixture was concentrated, dissolved in a minimum amount of DMSO and purified by reversed phase chromatography, eluting with a gradient of 20-80% 10 mM ammonium bicarbonate with 5% MeOH in ACN. Appropriate fractions were combined and were concentrated to low volume and were extracted with EtOAc. The organics were dried over anhydrous Na₂SO₄ and concentrated to obtain the product (0.010 g, 9%) as a brown solid. MS (ES+) m/z=482 (M+1).

### Preparation 62

### 7-Bromo-6-chloro-4-ethylsulfanyl-8-fluoro-quinazoline

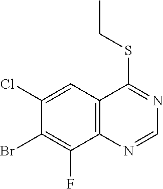 [SMILES: CCSc1ncnc2c(F)c(Br)c(Cl)cc12]

To a solution of 7-bromo-4,6-dichloro-8-fluoro-quinazoline (12.31 g, 41.60 mmol; see U.S. Pat. No. 9,840,516 B2, Column 355) in DCM (125 mL) was added ethanethiol (6.0 mL, 83.0 mmol) and was stirred at rt overnight. The reaction mixture was diluted with DCM, washed with sat. aq. NaHCO₃ (2×), dried over Na₂SO₄, filtered and concentrated. The residue was purified via silica gel chromatography, eluting with DCM and further recrystallized from hexanes and Et₂O to obtain the product (11.41 g, 85%) as an off-white solid. MS (ES+) m/z=323 (M+1).

### Preparation 63

### tert-Butyl N-[4-(6-chloro-4-ethylsulfanyl-8-fluoro-quinazolin-7-yl)-3-cyano-7-fluoro-benzothiophen-2-yl]carbamate

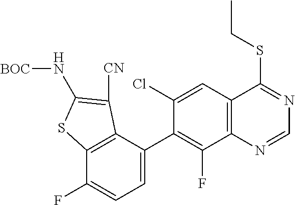 [SMILES: CCSc1ncnc2c(F)c(-c3ccc(F)c4sc(NC(=O)OC(C)(C)C)c(C#N)c34)c(Cl)cc12]

To a flask containing tert-butyl N-[3-cyano-4-(5,5-dimethyl-1,3,2-dioxaborinan-2-yl)-7-fluoro-benzothiophen-2-yl]carbamate (8.491 g, 18.90 mmol) was added 7-bromo-6-chloro-4-ethylsulfanyl-8-fluoro-quinazoline (5.694 g, 17.35 mmol) and toluene (70 mL). N₂ was bubbled through the solution while stirring and Cs₂CO₃ (11.31 g, 34.71 mmol) followed by dichlorobis(diphenylphosphinophenyl)ether palladium (II) (3.74 g, 5.22 mmol) were added. The reaction was heated at 110° C. overnight. The reaction mixture was diluted with EtOAc, filtered through diatomaceous earth and concentrated. The residue was purified via silica gel chromatography, eluting with a gradient of 0 to 75% acetone/hexanes to obtain the product (5.12 g, 53%) as a light-yellow solid. MS (ES+) m/z=533 (M+1).

### Preparation 64

### tert-Butyl 3-[7-[2-(tert-butoxycarbonylamino)-3-cyano-7-fluoro-benzothiophen-4-yl]-6-chloro-8-fluoro-quinazolin-4-yl]-3,8-diazabicyclo[3.2.1]octane-8-carboxylate

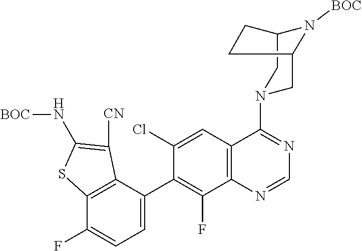 [SMILES: CC(C)(C)OC(=O)Nc1sc2c(F)ccc(-c3c(Cl)cc4c(N5CC6CCC(C5)N6C(=O)OC(C)(C)C)ncnc4c3F)c2c1C#N]

To a solution of tert-butyl N-[4-(6-chloro-4-ethylsulfanyl-8-fluoro-quinazolin-7-yl)-3-cyano-7-fluoro-benzothiophen-2-yl]carbamate (100 mg, 0.188 mmol) in DMF (1.88 mL) was added mCPBA (130 mg, 0.75 mmol) and was stirred at rt for 1 h. DMSO (0.27 mL, 3.75 mmol) was added and was stirred at rt for 5 min. A 0.3 M solution of tert-butyl 3,8-diazabicyclo[3.2.1]octane-8-carboxylate in DMF (2 mL, 0.60 mmol) and a 3M solution of TEA in DMF (0.31 mL, 0.93 mmol) were added and the reaction was stirred at rt for 1 h. A 10 g C18 SPE cartridge was equilibrated with 40 mL MeOH and 40 mL H₂O. The reaction mixture was poured onto the cartridge. The cartridge was eluted with 80 mL H₂O, 60 mL of a 3:1 mix of H₂O/MeOH, and 60 mL of a 1:1 mix of DCM/MeOH to afford the product (168 mg, 38% pure, 50% yield). MS (ES+) m/z=683 (M+1).

### Preparation 65

### tert-Butyl N-[4-(6-chloro-8-fluoro-4-hydroxy-quinazolin-7-yl)-3-cyano-7-fluoro-benzothiophen-2-yl]carbamate

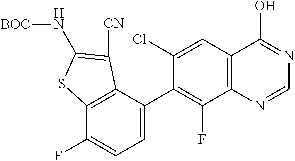 [SMILES: CC(C)(C)OC(=O)Nc1sc2c(F)ccc(-c3c(Cl)cc4c(O)ncnc4c3F)c2c1C#N]

To a solution of tert-butyl N-[4-(6-chloro-4-ethylsulfanyl-8-fluoro-quinazolin-7-yl)-3-cyano-7-fluoro-benzothiophen-2-yl]carbamate (3.50 g, 6.57 mmol) in acetone (75 mL) was added a solution of OXONE® (10.1 g, 16.4 mmol) in H₂O (60 mL) in one portion. THE (75 mL) was added, and the reaction mixture was stirred at rt overnight.

The reaction mixture was then concentrated and the residue was diluted with H₂O and EtOAc. The organic layer was separated, washed with sat. aq. NaCl and was dried over Na₂SO₄, filtered, and concentrated, to give the product (3.28 g, 100% yield) as a white solid. MS (ES+) m/z=489 (M+1).

### Preparation 66

### tert-Butyl 9-[7-[2-(tert-butoxycarbonylamino)-3-cyano-7-fluoro-benzothiophen-4-yl]-6-chloro-8-fluoro-quinazolin-4-yl]-3-oxa-7,9-diazabicyclo[3.3.1]nonane-7-carboxylate

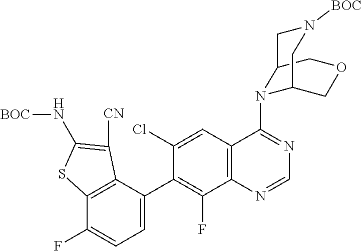 [SMILES: CC(C)(C)OC(=O)Nc1sc2c(F)ccc(-c3c(Cl)cc4c(N5C6COCC5CN(C(=O)OC(C)(C)C)C6)ncnc4c3F)c2c1C#N]

To a solution of tert-butyl N-[4-(6-chloro-8-fluoro-4-hydroxy-quinazolin-7-yl)-3-cyano-7-fluoro-benzothiophen-2-yl]carbamate (180 mg, 0.37 mmol) in ACN (10 mL) was added phosphonitrilic chloride trimer (128 mg, 0.37 mmol) and DIEA (0.32 mL, 1.84 mmol). After stirring at rt for 2 h, a 0.04 M solution of tert-butyl 3-oxa-7,9-diazabicyclo[3.3.1]nonane-7-carboxylate in ACN (9 mL, 0.36 mmol, 0.04M) and DIEA (0.26 mL, 1.47 mmol) were added. After stirring at rt for 6 h, the crude was dissolved with a mixture of DCM and EtOAc and washed with sat. aq. NaHCO₃ and sat. aq. NaCl. The organic layer was dried over Na₂SO₄, filtered and concentrated to afford the crude product (291 mg, 40% pure by LC-MS, 45% yield). MS (ES+) m/z=699 (M+1).

The compounds of Preparation 67 in Table 5 was prepared from 7-bromo-6-chloro-8-fluoroquinazolin-4-ol (see U.S. Pat. No. 9,840,516 B2, see Column 354 for 7-bromo-6-chloro-8-fluoroquinazolin-4(3H)-one) in a similar manner as described in Preparation 66.

TABLE 5
Compounds of Preparation 67.

| | | | MS |
|---|---|---|---|
| | | | (ES) |
| | Chemical | | m/z |
| Preparation | Name | Structure | (M + H) |
| 67 | tert-Butyl 8- (7-bromo-6- chloro-8- fluoro- quinazolin- 4-yl)-3,8- diazabicyclo [3.2.1]octane- 3-carboxylate | 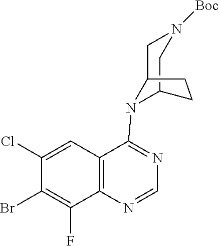 [SMILES: CC(C)(C)OC(=O)N1CC2CCC(C1)N2c1ncnc2c(F)c(Br)c(Cl)cc12] | 471 |

### Preparation 68

### tert-Butyl (1S,4S)-5-(7-bromo-6-chloro-8-fluoro-quinazolin-4-yl)-2,5-diazabicyclo[2.2.1]heptane-2-carboxylate

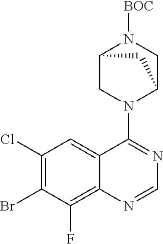 [SMILES: CC(C)(C)OC(=O)N1C[C@@H]2C[C@H]1CN2c1ncnc2c(F)c(Br)c(Cl)cc12]

A mixture of 7-bromo-4,6-dichloro-8-fluoro-quinazoline (250 mg, 0.844 mmol, see U.S. Pat. No. 9,840,516 B2, Column 355), tert-butyl (1S,4S)-2,5-diazabicyclo[2.2.1]heptane-2-carboxylate (184 mg, 0.928 mmol), DIEA (0.44 mL, 2.5 mmol) in ACN (5.6 mL) was heated to 60° C. for 1 h. The reaction mixture was partially concentrated to about half of original volume and the solid was collected via vacuum filtration and was washed with minimal Et₂O. The resulting material was dried in vacuum oven overnight to give the product (284 mg, 74% yield) as a beige solid. MS (ES+) m/z=459 (M+1).

The compound of Preparation 69 in Table 6 was prepared in a similar manner as described in Preparation 68.

TABLE 6
Compounds of Preparation 69.

| | | | MS |
|---|---|---|---|
| | | | (ES) |
| | Chemical | | m/z |
| Preparation | Name | Structure | (M + H) |
| 69 | tert-Butyl 3-(7- bromo-6- chloro-8- fluoro- quinazolin- 4-yl)-3,6- diazabicyclo [3.1.1] heptane-6- carboxylate | 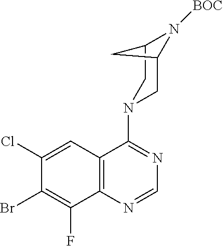 [SMILES: CC(C)(C)OC(=O)N1C2CC1CN(c1ncnc3c(F)c(Br)c(Cl)cc13)C2] | 459 |

### Preparation 70

### O1-tert-Butyl O3-methyl 3-(7-bromo-6-chloro-8-fluoro-quinazolin-4-yl)azetidine-1,3-dicarboxylate

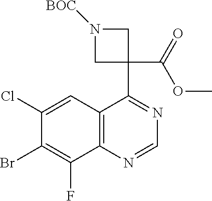 [SMILES: COC(=O)C1(c2ncnc3c(F)c(Br)c(Cl)cc23)CN(C(=O)OC(C)(C)C)C1]

To a cooled solution of 7-bromo-4,6-dichloro-8-fluoro-quinazoline (1.06 g, 3.58 mmol) and O1-tert-butyl O3-methyl azetidine-1,3-dicarboxylate (980 mg, 4.55 mmol) in THE (15 mL) in a −78° C. dry ice/acetone bath was added 1M lithium bis(trimethylsilyl)amide in THE (4 mL, 4 mmol, 1 mol/L) dropwise. After 10 min., the reaction was quenched by the addition of sat. aq. NH₄Cl solution and then was extracted 3× with EtOAc. The combined organic layers were dried over MgSO₄, filtered and concentrated. The residue was purified via silica gel chromatography, eluting with a gradient of 0 to 60% EtOAc/hexanes to obtain the product (810 mg, 43% yield) as an off-white solid. MS (ES+) m/z=474 (M+1).

### Preparation 71

### tert-Butyl 3-(7-bromo-6-chloro-8-fluoro-quinazolin-4-yl)azetidine-1-carboxylate

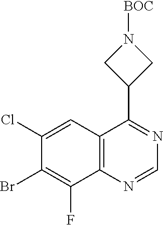 [SMILES: CC(C)(C)OC(=O)N1CC(c2ncnc3c(F)c(Br)c(Cl)cc23)C1]

A microwave vessel was charged with O1-tert-butyl O3-methyl 3-(7-bromo-6-chloro-8-fluoro-quinazolin-4-yl)azetidine-1,3-dicarboxylate (400 mg, 0.7584 mmol), LiCl (340 mg, 8.02 mmol) and DMSO (2 mL). The vessel then was placed in a microwave reactor at 150° C. for 5 min. To the reaction mixture was added H₂O and then the mixture was extracted 2× with EtOAc. The combined organic layers were washed with H₂O, sat. aq. NaCl, and were dried over MgSO₄, filtered, and concentrated. The residue was purified via silica gel chromatography, eluting with a gradient of 0-60% EtOAc/hexanes to obtain the product (294 mg, 83% yield) as a light-yellow solid. MS (ES+) m/z=416 (M+1).

The compounds of Preparations 72 and 73 in Table 7 were prepared in a similar manner as described in Preparations 70 and 71. Different methods were used for purifying the molecules, which would be apparent to one skilled in the art.

TABLE 7
Compounds of Preparations 72 and 73.

| | | | MS (ES) |
|---|---|---|---|
| Preparation | Chemical Name | Structure | m/z (M − H) |
| 72 | tert-Butyl 3-(7- bromo-6- chloro-8-fluoro- quinazolin-4- yl)pyrrolidine-1- carboxylate | 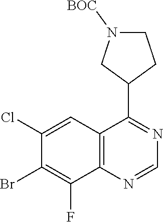 [SMILES: CC(C)(C)OC(=O)N1CCC(c2ncnc3c(F)c(Br)c(Cl)cc23)C1] | 428 |
| 73 | tert-Butyl 3-(7- bromo-6- chloro-8-fluoro- quinazolin-4-yl) piperidine- 1-carboxylate | 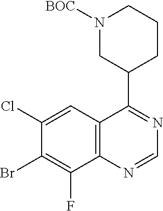 [SMILES: CC(C)(C)OC(=O)N1CCCC(c2ncnc3c(F)c(Br)c(Cl)cc23)C1] | 442 |

The compounds of Preparations 74 to 80 in Table 8 were prepared in a similar manner as described in Preparation 63. Different methods were used for purifying the molecules, which would be apparent to one skilled in the art.

TABLE 8
Compounds of Preparations 74 to 80.

| | | | MS |
|---|---|---|---|
| | | | (ES) m/z |
| Preparation | Chemical Name | Structure | (M + H) |
| 74 | tert-Butyl 4-[7-[2-(tert- butoxycarbonylamino)- 3-cyano-7-fluoro- benzothiophen-4-yl]-6- chloro-8-fluoro- quinazolin-4- yl]piperazine-1- carboxylate | 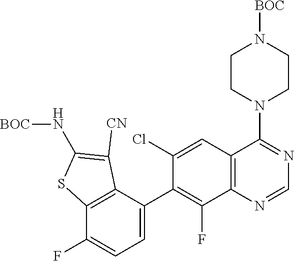 [SMILES: CC(C)(C)OC(=O)Nc1sc2c(F)ccc(-c3c(Cl)cc4c(N5CCN(C(=O)OC(C)(C)C)CC5)ncnc4c3F)c2c1C#N] | 657 |
| 75 | tert-Butyl 8-[7-[2-(tert- butoxycarbonylamino)-3- cyano-7-fluoro- benzothiophen-4-yl]-6- chloro-8-fluoro- quinazolin-4-yl]-3,8- diazabicyclo [3.2.1 ]octane- 3-carboxylate | 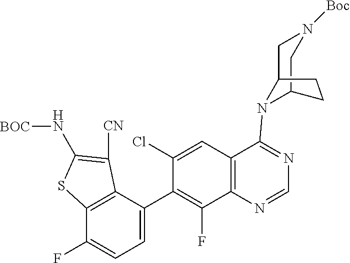 [SMILES: CC(C)(C)OC(=O)Nc1sc2c(F)ccc(-c3c(Cl)cc4c(N5C6CCC5CN(C(=O)OC(C)(C)C)C6)ncnc4c3F)c2c1C#N] | 683 |
| 76 | tert-Butyl (1S, 4S)-5-[7- [2-(tert- butoxycarbonylamino)-3- cyano-7-fluoro- benzothiophen-4-yl]-6- chloro-8-fluoro- quinazolin-4-yl]-2,5- diazabicyclo[2.2.1 ] heptane-2-carboxylate | 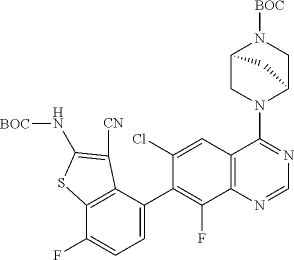 [SMILES: CC(C)(C)OC(=O)Nc1sc2c(F)ccc(-c3c(Cl)cc4c(N5C[C@@H]6C[C@H]5CN6C(=O)OC(C)(C)C)ncnc4c3F)c2c1C#N] | 669 |
| 77 | tert-Butyl 3-[7-[2-(tert- butoxycarbonylamino)-3- cyano-7-fluoro- benzothiophen-4-yl]-6- chloro-8-fluoro- quinazolin-4-yl]-3,6- diazabicyclo[3.1.1] heptane- 6-carboxylate | 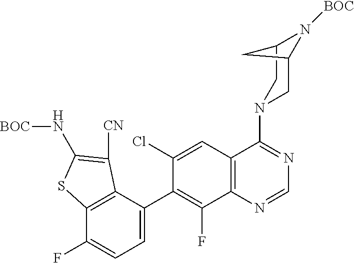 [SMILES: CC(C)(C)OC(=O)Nc1sc2c(F)ccc(-c3c(Cl)cc4c(N5CC6CC(C5)N6C(=O)OC(C)(C)C)ncnc4c3F)c2c1C#N] | 669 |
| 78 | tert-Butyl 3-[7-[2-(tert- butoxycarbonylamino)-3- cyano-7-fluoro- benzothiophen-4-yl]-6- chloro-8-fluoro- quinazolin-4-yl]azetidine- 1-carboxylate | 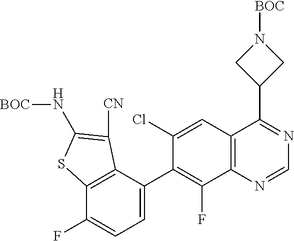 [SMILES: CC(C)(C)OC(=O)Nc1sc2c(F)ccc(-c3c(Cl)cc4c(C5CN(C(=O)OC(C)(C)C)C5)ncnc4c3F)c2c1C#N] | 628 |
| 79 | tert-Butyl 3-[7-[2-(tert- butoxycarbonylamino)-3- cyano-7-fluoro- benzothiophen-4-yl]-6- chloro-8-fluoro- quinazolin-4- yl]pyrrolidine-1- carboxylate | 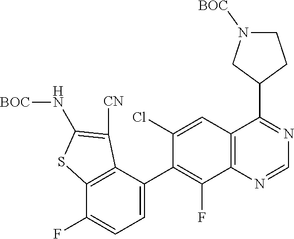 [SMILES: CC(C)(C)OC(=O)Nc1sc2c(F)ccc(-c3c(Cl)cc4c(C5CCN(C(=O)OC(C)(C)C)C5)ncnc4c3F)c2c1C#N] | 642 |
| 80 | tert-Butyl 3-[7-[2-(tert- butoxycarbonylamino)-3- cyano-7-fluoro- benzothiophen-4-yl]-6- chloro-8-fluoro- quinazolin-4- yl]piperidine-1- carboxylate | 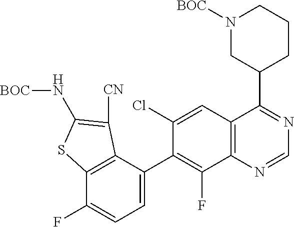 [SMILES: CC(C)(C)OC(=O)Nc1sc2c(F)ccc(-c3c(Cl)cc4c(C5CCCN(C(=O)OC(C)(C)C)C5)ncnc4c3F)c2c1C#N] | 656 |

The Example compounds of Table 9 were prepared in a similar manner as described in Example 13. Various methods were used to purify the compounds, which would be apparent to one skilled in the art.

TABLE 9
Example Compounds 32 to 40b.

| | | | MS |
|---|---|---|---|
| | | | (ES) m/z |
| Example | Chemical Name | Structure | (M + H) |
| 32 | 2-Amino-4-(6-chloro-8- fluoro-4-piperazin-1-yl- quinazolin-7-yl)-7-fluoro- benzothiophene-3- carbonitrile | 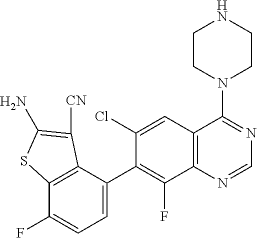 [SMILES: N#Cc1c(N)sc2c(F)ccc(-c3c(Cl)cc4c(N5CCNCC5)ncnc4c3F)c12] | 457 |
| 33 | 2-Amino-4-[6-chloro-4- (3,8- diazabicyclo[3.2.1]octan- 3-yl)-8-fluoro-quinazolin- 7-yl]-7-fluoro- benzothiophene-3- carbonitrile | 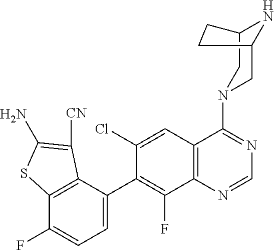 [SMILES: N#Cc1c(N)sc2c(F)ccc(-c3c(Cl)cc4c(N5CC6CCC(C5)N6)ncnc4c3F)c12] | 483 |
| 34 | 2-Amino-4-[6-chloro-8- fluoro-4-(3-oxa-7,9- diazabicyclo[3.3.1]nonan- 9-yl)quinazolin-7-yl]-7- fluoro-benzothiophene-3- carbonitrile | 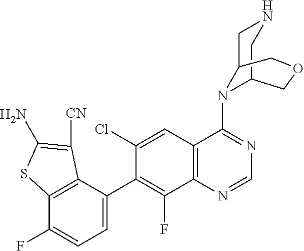 [SMILES: N#Cc1c(N)sc2c(F)ccc(-c3c(Cl)cc4c(N5C6CNCC5COC6)ncnc4c3F)c12] | 499 |
| 35 | 2-Amino-4-[6-chloro-4- (3,8- diazabicyclo[3.2.1]octan- 8-yl)-8-fluoro-quinazolin- 7-yl]-7-fluoro- benzothiophene-3- carbonitrile | 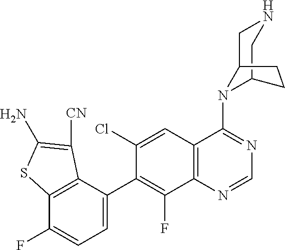 [SMILES: N#Cc1c(N)sc2c(F)ccc(-c3c(Cl)cc4c(N5C6CCC5CNC6)ncnc4c3F)c12] | 483 |
| 36 | 2-Amino-4-[6-chloro-4- [(1S,4S)-2,5- diazabicyclo[2.2.1]heptan- 2-yl]-8-fluoro- quinazolin-7-yl]-7-fluoro- benzothiophene-3- carbonitrile (isomer 1) | 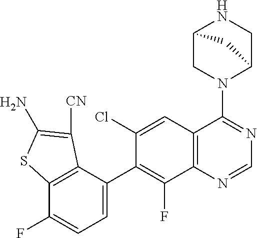 [SMILES: N#Cc1c(N)sc2c(F)ccc(-c3c(Cl)cc4c(N5C[C@@H]6C[C@H]5CN6)ncnc4c3F)c12] | 469 |
| 37 | 2-Amino-4-[6-chloro-4- (3,6- diazabicyclo[3.1.1] heptan- 3-yl)-8-fluoro- quinazolin-7-yl]-7-fluoro- benzothiophene-3- carbonitrile | 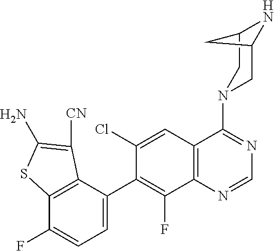 [SMILES: N#Cc1c(N)sc2c(F)ccc(-c3c(Cl)cc4c(N5CC6CC(C5)N6)ncnc4c3F)c12] | 469 |
| 38 | 2-Amino-4-[4-(azetidin- 3-yl)-6-chloro-8-fluoro- quinazolin-7-yl]-7-fluoro- benzothiophene-3- carbonitrile | 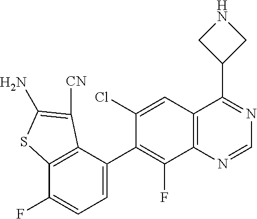 [SMILES: N#Cc1c(N)sc2c(F)ccc(-c3c(Cl)cc4c(C5CNC5)ncnc4c3F)c12] | 428 |
| 39 | 2-Amino-4-[6-chloro-8- fluoro-4-[pyrrolidin-3- yl]quinazolin-7-yl]-7- fluoro-benzothiophene-3- carbonitrile, isomer 1 | 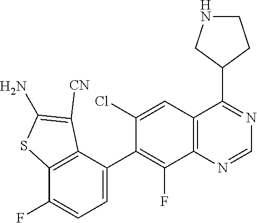 [SMILES: N#Cc1c(N)sc2c(F)ccc(-c3c(Cl)cc4c(C5CCNC5)ncnc4c3F)c12] | 442 |
| 40a | 2-Amino-4-[6-chloro-8- fluoro-4-[(3R)-3- piperidyl]quinazolin-7- yl]-7-fluoro- benzothiophene-3- carbonitrile, diastereomer 1 | 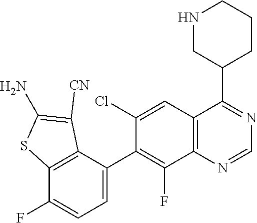 [SMILES: N#Cc1c(N)sc2c(F)ccc(-c3c(Cl)cc4c(C5CCCNC5)ncnc4c3F)c12] | 456 |
| 40b | 2-Amino-4-[6-chloro-8- fluoro-4-[(3R)-3- piperidyl]quinazolin-7- yl]-7-fluoro- benzothiophene-3- carbonitrile, diastereomer 2 | 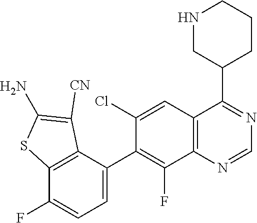 [SMILES: N#Cc1c(N)sc2c(F)ccc(-c3c(Cl)cc4c(C5CCCNC5)ncnc4c3F)c12] | 456 |

### Preparation 81

### 6-Bromo-7-chloro-2,8-difluoro-quinoline

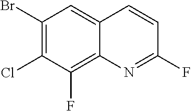 [SMILES: Fc1ccc2cc(Br)c(Cl)c(F)c2n1]

In a glove box under an atmosphere of N₂ in an oven-dried flask, a slurry of 6-bromo-7-chloro-8-fluoro-quinoline (4.31 g, 16.5 mmol) in anhydrous ACN (160 mL) was treated with AgF₂ (7.54 g, 51.7 mmol). The resulting mixture was stirred overnight at ambient temperature in the glove box. The mixture was filtered through a pad of diatomaceous earth and the solids were rinsed with DCM. The filtrate was concentrated in vacuo to give an orange solid which was stirred in 100 mL of DCM for several minutes before filtering to remove the remaining solids. The filtrate was purified directly on silica (eluting with DCM) to give the product (2.64 g, 57%) as a white solid. GC/MS (m z): 277.0 (M+).

### Preparation 82

### 6-Bromo-7-chloro-8-fluoro-2-[[(2S)-1-methylpyrrolidin-2-yl]methoxy]quinoline

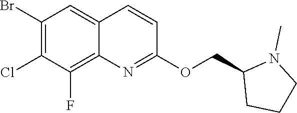 [SMILES: CN1CCC[C@H]1COc1ccc2cc(Br)c(Cl)c(F)c2n1]

Under an atmosphere of N₂, a mixture of N-methyl-L-prolinol (2.4 mL, 20 mmol) and THE (120 mL) was treated with a solution of lithium bis(trimethylsilyl)amide (1.3 mol/L) in THE (16 mL, 21 mmol) dropwise via syringe. The resulting mixture was stirred for 20 min. Solid 6-bromo-7-chloro-2,8-difluoro-quinoline (6.30 g, 17.0 mmol, 75% purity) was added in one portion and the mixture was stirred overnight at ambient temperature. The reaction mixture was quenched with H₂O and diluted with EtOAc. The layers were separated and the organic layer was washed with sat. aq. NaCl, dried over MgSO₄, filtered, and concentrated in vacuo. The residue was purified on silica (eluting with a gradient of 0 to 10% MeOH in EtOAc) to give the product (5.61 g, 88.5%). ES/MS (m z): 373.0 (M+H).

### Preparation 83

### 7-Chloro-8-fluoro-6-methyl-2-[[(2S)-1-methylpyrrolidin-2-yl]methoxy]quinoline

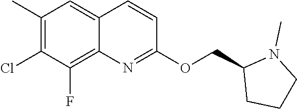 [SMILES: Cc1cc2ccc(OC[C@@H]3CCCN3C)nc2c(F)c1Cl]

A mixture of 6-bromo-7-chloro-8-fluoro-2-[[(2S)-1-methylpyrrolidin-2-yl]methoxy]quinoline (0.45 g, 1.2 mmol), K₂CO₃ (0.47 g, 3.4 mmol), 1,4-dioxane (8 mL), and trimethylboroxine (0.20 mL, 1.4 mmol) in a microwave reaction vessel was degassed with N₂. Tetrakis(triphenylphosphine)palladium(0) (0.072 g, 0.060 mmol) was added. The resulting mixture was heated in a BIOTAGE INITIATOR® microwave reactor at 120° C. for 0.5 h, then it was filtered through a pad of diatomaceous earth and was rinsed with EtOAc. The filtrate was concentrated in vacuo and the residue was purified on silica (eluting with a gradient of 2.5% MeOH/DCM to 5% MeOH/DCM to 10% MeOH/DCM) to give the product (0.239 g, 64%). ES/MS (m z): 309.2 (M+H).

### Preparation 84

### 7-Chloro-6-cyclopropyl-8-fluoro-2-[[(2S)-1-methylpyrrolidin-2-yl]methoxy]quinoline

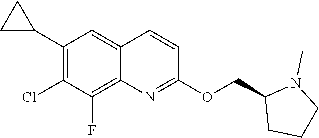 [SMILES: CN1CCC[C@H]1COc1ccc2cc(C3CC3)c(Cl)c(F)c2n1]

A mixture of 6-bromo-7-chloro-8-fluoro-2-[[(2S)-1-methylpyrrolidin-2-yl]methoxy]quinoline (0.200 g, 0.535 mmol), cyclopropylboronic acid (0.092 mg, 1.07 mmol), tetrakis(triphenylphosphine)palladium(0) (0.062 g, 0.054 mmol), K₃PO₄ (0.239 g, 1.07 mmol) and 1,4-dioxane (5.35 mL) was heated at 90° C. overnight. The reaction mixture was cooled and filtered over filter paper. The filtrate was concentrated in vacuo. A second batch was run starting with 50 mg of 6-bromo-7-chloro-8-fluoro-2-[[(2S)-1-methylpyrrolidin-2-yl]methoxy]quinoline in the same manner as described. The residues from each reaction were dissolved in MeOH, combined and purified on SCX, rinsing first with MeOH, followed by ammoniated methanol elution to afford the product (196 mg, 87%). ES/MS (m z): 335.0 (M+H).

### Example 41

### 2-Amino-7-fluoro-4-[8-fluoro-6-methyl-2-[[(2S)-1-methylpyrrolidin-2-yl]methoxy]-7-quinolyl]benzothiophene-3-carbonitrile

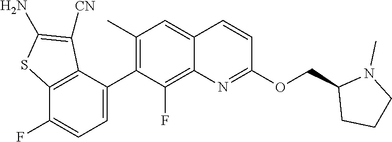 [SMILES: Cc1cc2ccc(OC[C@@H]3CCCN3C)nc2c(F)c1-c1ccc(F)c2sc(N)c(C#N)c12]

A mixture of KOtBu (0.067 g, 0.60 mmol), tert-butyl N-[3-cyano-4-(5,5-dimethyl-1,3,2-dioxaborinan-2-yl)-7-fluoro-benzothiophen-2-yl]carbamate (0.213 g, 0.527 mmol), 7-chloro-8-fluoro-6-methyl-2-[[(2S)-1-methylpyrrolidin-2-yl]methoxy]quinoline (0.116 g, 0.376 mmol) in THE (5 mL) was degassed with N₂. SPhos Pd(crotyl)Cl (Pd-172; CAS #1798781-99-3) (0.057 g, 0.09383 mmol) was added and the resulting mixture was heated at 70° C. overnight. The reaction mixture was cooled to ambient temperature and was diluted with EtOAc and H₂O. The layers were separated. The organic layer was washed with sat. aq. NaCl, dried over MgSO₄, filtered and concentrated in vacuo. The residue was purified on silica, eluting with a gradient of 2.5% to 5% MeOH in DCM. The product containing fractions were combined and concentrated in vacuo. The residue was dissolved in DCM (5 mL), and TFA (0.5 mL) was added. The resulting mixture was stirred at rt for 4 h and then was warmed at 40° C. for 1.5 h before concentrating in vacuo. The residue was dissolved in MeOH and loaded onto a 10 g SCX cartridge, which was pre-washed with MeOH. The column was eluted with methanol, followed by 2M ammonia in methanol. The ammoniated eluent was concentrated in vacuo. The residue was purified on silica (eluting with a gradient of 2.5% MeOH:DCM to 5% MeOH:DCM to 10% MeOH:DCM to 10% 2M ammonia in MeOH:DCM) to give the product as a white solid (27 mg, 15%). ES/MS (m z): 465.2 (M+H).

### Example 42

### 2-Amino-4-[6-cyclopropyl-8-fluoro-2-[[(2S)-1-methylpyrrolidin-2-yl]methoxy]-7-quinolyl]-7-fluoro-benzothiophene-3-carbonitrile

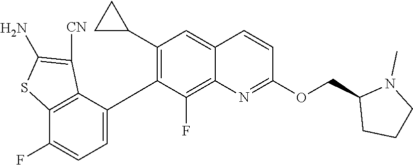 [SMILES: CN1CCC[C@H]1COc1ccc2cc(C3CC3)c(-c3ccc(F)c4sc(N)c(C#N)c34)c(F)c2n1]

Prepared from 7-chloro-6-cyclopropyl-8-fluoro-2-[[(2S)-1-methylpyrrolidin-2-yl]methoxy]quinoline using XPhos Pd(crotyl)Cl (Pd-170; CAS #1798782-02-1) as the catalyst in a manner analogous to the method of Example 41 to afford the product (0.062 g, 22%). ES/MS (m z): 491.2 (M+H).

### Preparation 85

### Methyl 2-amino-3-chloro-5,6-difluoro-benzoate

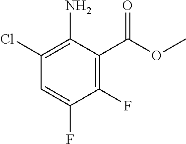

A solution of methyl 6-amino-2,3-difluoro-benzoate (63.3 g, 338 mmol) in acetonitrile (1.2 L) was treated with NCS (50 g, 374 mmol). The resulting mixture was heated at 45° C. overnight. The reaction was concentrated to half volume and sat. aq. NaHCO₃ was added. The mixture was diluted with EtOAc, and the layers were separated. The organic phase was washed with sat. aq. NaCl solution, dried over Na₂SO₄, and concentrated in vacuo. The residue was purified by flash silica gel chromatography (eluting with a gradient of 10 to 25% EtOAc in hexanes) to give the product (49.5 g, 66%). ¹H NMR (399.80 MHz, CDCl₃): δ 7.32 (dd, J=7.6, 9.4 Hz, 1H), 6.00 (br s, 2H), 3.97 (s, 3H).

### Preparation 86

### Methyl 2-bromo-3-chloro-5,6-difluoro-benzoate

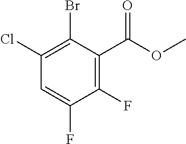

A mixture of CuBr₂ (105 g, 470 mmol) and tert-butyl nitrite (78 mL, 590 mmol) in ACN (400 mL) was cooled in an ice-water bath and was treated with a solution of methyl 2-amino-3-chloro-5,6-difluoro-benzoate (91.2 g, 412 mmol) in ACN (400 mL) dropwise over 15 min. The resulting mixture was allowed to warm slowly to ambient temperature and was stirred overnight. The reaction was concentrated to half volume, diluted with DCM (2 L), and allowed to stand for 5 h. The mixture was filtered through pad of diatomaceous earth and was rinsed with DCM (1 L), followed by a mixture of 10% EtOAc/DCM until the filtrate was nearly colorless. The combined filtrate was washed twice with 10% citric acid (500 mL), twice with H₂O (500 mL) and once with sat. aq. EDTA solution (500 mL) before concentrating in vacuo. The resulting solid was purified by flash silica gel chromatography (eluting with a gradient of 10 to 40% EtOAc in hexanes) to give the product (122 g, quantitative). ¹H NMR (399.80 MHz, CDCl₃): δ 7.44 (dd, J=7.4, 9.4 Hz, 1H), 4.02 (s, 3H).

### Preparation 87

### Methyl 5-chloro-2,3-difluoro-6-(methoxymethyl)benzoate

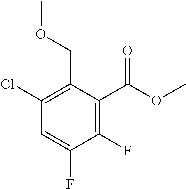 [SMILES: COCc1c(Cl)cc(F)c(F)c1C(=O)OC]

A mixture of methyl 2-bromo-3-chloro-5,6-difluoro-benzoate (25.0 g, 85.8 mmol) and potassium (methoxymethyl)trifluoroborate (19.5 g, 128 mmol) in dioxane (450 mL) was treated with a mixture of Cs₂CO₃ (84 g, 258 mmol) in H₂O (45 mL) was successively evacuated and refilled with N₂ five times. [1,1′-Bis(diphenylphosphino)ferrocene]dichloropalladium(II), Pd(dppf)Cl₂ (6.27 g, 8.57 mmol) was added, and the resulting mixture was vacuum degassed and purged with N₂ five additional times before heating at 100° C. overnight. Additional potassium (methoxymethyl)trifluoroborate (5.0 g, 32.9 mmol), Cs₂CO₃ (21 g, 64.5 mmol) in H₂O (10 mL) and [1,1′-bis(diphenylphosphino)ferrocene]dichloropalladium(II), Pd(dppf)Cl₂ (1.6 g, 2.2 mmol) was added and heating was resumed for 12 h. The mixture was cooled to ambient temperature, diluted with EtOAc (1.5 L), H₂O (1 L) and sat. aq. NaCl (1 L). The layers were separated and the aqueous layer was extracted with EtOAc (1 L). The organic layers were combined and concentrated in vacuo. The residue was purified by silica gel chromatography (eluting with a gradient of 0 to 15% EtOAc in hexanes) to give the product (15 g, 70%) as a slightly colored oil. ES/MS (m/z): 251.2 (M+H).

### Preparation 88

### 5-Chloro-2,3-difluoro-6-(methoxymethyl)benzoic acid

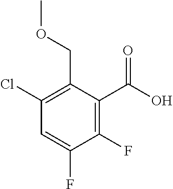

A solution of methyl 5-chloro-2,3-difluoro-6-(methoxymethyl)benzoate (10 g, 39.9 mmol) in THE (100 mL) and MeOH (65 mL) was treated with 5M aq. NaOH (24 mL, 120 mmol). The resulting mixture was stirred overnight at ambient temperature. Ice chips were added, and the cold slush was treated with 5N aq. HCl in a dropwise fashion to adjust to pH ˜1-2. The mixture was extracted with EtOAc (3×500 mL). The organic layers were combined, dried over Na₂SO₄, filtered and concentrated in vacuo. The solid was dried in a vacuum oven at 55° C. for 2 h to give the product (9.49 g, 95.5%). ES/MS (m z): 237.0 (M+H).

### Preparation 89

### 5-Chloro-N-(ethylsulfanylcarbonimidoyl)-2,3-difluoro-6-(methoxymethyl)benzamide

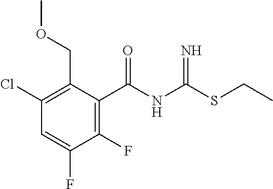 [SMILES: CCSC(=N)NC(=O)c1c(F)c(F)cc(Cl)c1COC]

A suspension of 5-chloro-2,3-difluoro-6-(methoxymethyl)benzoic acid (9.49 g, 40.1 mmol) and DIEA (28 mL, 161 mmol) in THE (350 mL) was cooled in an ice-water bath. S-Ethylisothiourea hydrobromide (12.0 g, 63.5 mmol) was added followed by HATU (24.0 g, 61.9 mmol). The resulting mixture was allowed to slowly warm to ambient temperature as the ice bath melted. The reaction mixture was diluted with EtOAc and washed with sat. aq. NaHCO₃ and sat. aq. NaCl. The organic layer was dried over Na₂SO₄, filtered, and concentrated in vacuo. The oily residue was purified by silica gel chromatography (eluting with DCM) to give the product (12.9 g, 99%) as a clear, colorless oil. ES/MS (m/z): 323.2 (M+H).

### Preparation 90

### 6-Chloro-2-ethylsulfanyl-8-fluoro-5-(methoxymethyl)quinazolin-4-ol

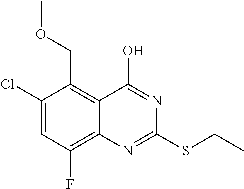 [SMILES: CCSc1nc(O)c2c(COC)c(Cl)cc(F)c2n1]

A solution of 5-chloro-N-(ethylsulfanylcarbonimidoyl)-2,3-difluoro-6-(methoxymethyl)benzamide (3.2 g, 9.4 mmol, 95% purity) in NMP (25 mL) was heated at 100° C. overnight. The reaction mixture was poured into cooled deionized water (600 mL). More water was added and the solids were collected by filtration. The solids were rinsed with additional H₂O (500 mL) and were dried in the vacuum oven at 55° C. to give the product (2.84 g, 99%) as a light-tan solid. ES/MS (m z): 303.2 (M+H).

### Preparation 91

### 6-Chloro-2-ethylsulfanyl-8-fluoro-7-iodo-5-(methoxymethyl)quinazolin-4-ol

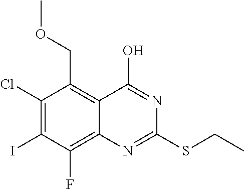 [SMILES: CCSc1nc(O)c2c(COC)c(Cl)c(I)c(F)c2n1]

6-Chloro-2-ethylsulfanyl-8-fluoro-5-(methoxymethyl)-3H-quinazolin-4-one (3.50 g, 11.4 mmol) was placed in a dry 250 mL 3-necked round bottom flask equipped with a thermocouple and a dropping funnel. The flask was sealed and flushed with N₂. Anhydrous THF (60 mL) was added via cannula and the mixture was heated to 60° C. 2,2,6,6-Tetramethylpiperidinylzinc chloride lithium chloride complex (1M in THF, 35 mL, 35 mmol) was added dropwise over 5 min. to the reaction mixture. After 2 h, the reaction mixture was treated with additional 2,2,6,6-tetramethylpiperidinylzinc chloride-lithium chloride complex (1M in THF, 11 mL, 11 mmol) dropwise and heating at 60° C. was continued overnight. Solid I₂ (5.8 g, 23 mmol) was added in several small portions at such a rate to keep the internal temperature under 70° C. The reaction was heated for another 5 h. After cooling to ambient temperature, the reaction mixture was diluted with EtOAc (100 mL) and 1N aq. HCl (100 mL). The layers were separated and the organic layer was dried over Na₂SO₄, filtered and concentrated in vacuo. DCM (100 mL) was added and the mixture was stirred for 10 min. The solids were collected by filtration and were rinsed with additional DCM (20 mL) to give the product (2.96 g, 55.5%, 92% purity). The filtrate was concentrated in vacuo, and the residue was purified by silica gel chromatography (eluting with 25% EtOAc in hexanes) to give additional batch of product (0.910 g, 17%, 85% purity) as a white solid. ES/MS (m/z): 429.4 (M+H).

### Preparation 92

### 4,6-Dichloro-2-ethylsulfanyl-8-fluoro-7-iodo-5-(methoxymethyl)quinazoline

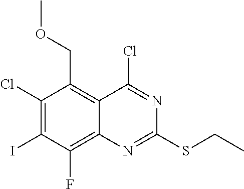 [SMILES: CCSc1nc(Cl)c2c(COC)c(Cl)c(I)c(F)c2n1]

A suspension of 6-chloro-2-ethylsulfanyl-8-fluoro-7-iodo-5-(methoxymethyl)quinazolin-4-ol (5.08 g, 11.9 mmol) in DCM (100 mL) was charged with (chloromethylene)dimethyliminium chloride (3.03 g, 23.7 mmol) in one portion. The resulting mixture was stirred for 2 h at ambient temperature. Additional (chloromethylene)dimethyliminium chloride (0.30 g, 2.3 mmol) was added and was stirred overnight. The reaction mixture was diluted with DCM and washed with H₂O three times. The organic layer was dried over Na₂SO₄ and MgSO₄, filtered, and concentrated in vacuo. The resulting brown solid was dissolved in DCM and treated with activated charcoal (DARCO® 20-40 mesh). The mixture was stirred vigorously for 5 min. The clear solution was filtered through a plug of silica, eluting with DCM. The filtrate was concentrated in vacuo and further dried in the vacuum oven at 45° C. to give the product (4.4 g, 76%) as a slightly yellow solid. ES/MS (m z): 447.2 (M+H).

### Preparation 93

### 6-Chloro-2-ethylsulfanyl-8-fluoro-7-iodo-5-(methoxymethyl)quinazoline

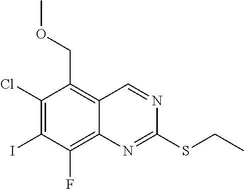 [SMILES: CCSc1ncc2c(COC)c(Cl)c(I)c(F)c2n1]

In a sealed reaction vessel with a Teflon screw cap, a mixture of 4,6-dichloro-2-ethylsulfanyl-8-fluoro-7-iodo-5-(methoxymethyl)quinazoline (1.5 g, 3.4 mmol) and p-toluenesulfonyl hydrazide (1.9 g, 10 mmol) in CHCl₃ (70 mL, 874 mmol) was heated at 55° C. overnight. The white suspension was evaporated to dryness using a stream of N₂ to obtain 1.85 g of N′-[6-chloro-2-ethylsulfanyl-8-fluoro-7-iodo-5-(methoxymethyl)quinazolin-4-yl]-4-methyl-benzenesulfonohydrazide. To this solid was added a solution of Na₂CO₃ (3.28 g, 31.0 mmol) in H₂O (83 mL). The mixture was heated in a sealed reaction vessel at 120° C. for 6.5 h. The reaction was filtered and the solids were rinsed with deionized water until the pH of the filtrate was ˜7-8. The resulting tan solid was purified by silica gel chromatography (eluting with a gradient of 0 to 5% MeOH in DCM) to give the product (1.0 g, 72% overall yield) as a light-yellow solid. ES/MS (m z): 412.6 (M+H).

### Preparation 94

### tert-Butyl N-[4-[6-chloro-2-ethylsulfanyl-8-fluoro-5-(methoxymethyl)quinazolin-7-yl]-3-cyano-7-fluoro-benzothiophen-2-yl]carbamate

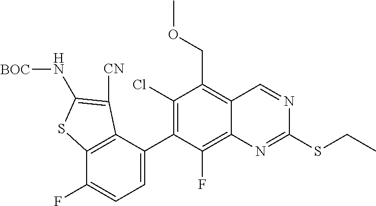 [SMILES: CCSc1ncc2c(COC)c(Cl)c(-c3ccc(F)c4sc(NC(=O)OC(C)(C)C)c(C#N)c34)c(F)c2n1]

Prepared from 6-chloro-2-ethylsulfanyl-8-fluoro-7-iodo-5-(methoxymethyl)quinazoline in a manner analogous to the method of Preparation 36 to give the product (1.03 g, 71.7%) as a tan solid. ES/MS (m z): 491.2 (M+H).

### Preparation 95

### tert-Butyl N-[4-[6-chloro-2-ethylsulfonyl-8-fluoro-5-(methoxymethyl)quinazolin-7-yl]-3-cyano-7-fluoro-benzothiophen-2-yl]carbamate

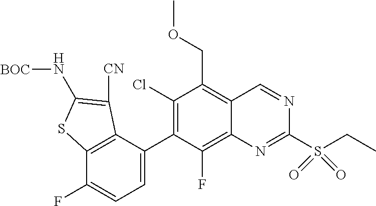 [SMILES: CCS(=O)(=O)c1ncc2c(COC)c(Cl)c(-c3ccc(F)c4sc(NC(=O)OC(C)(C)C)c(C#N)c34)c(F)c2n1]

A mixture of tert-butyl N-[4-[6-chloro-2-ethylsulfanyl-8-fluoro-5-(methoxymethyl)quinazolin-7-yl]-3-cyano-7-fluoro-benzothiophen-2-yl]carbamate (0.500 g, 0.87 mmol) in DCM (20 mL) was treated with mCPBA (0.530 g, 2.36 mmol, 77%). The resulting mixture was stirred at ambient temperature for 1.5 h. The reaction mixture was diluted with DCM and washed with 1M sat. aq. Na₂S₂O₃ and sat. aq. NaHCO₃. The layers were separated, the organic layer dried over Na₂SO₄, filtered and concentrated in vacuo. The residue was purified by silica gel chromatography (eluting with a gradient from 10 to 70% EtOAc in hexanes) to give the product (0.502 g, 95%) as a tan solid. ES/MS (m/z): 609.2 (M+H).

### Preparation 96

### tert-Butyl N-[4-[6-chloro-8-fluoro-5-(methoxymethyl)-2-[[(2S)-1-methylpyrrolidin-2-yl]methoxy]quinazolin-7-yl]-3-cyano-7-fluoro-benzothiophen-2-yl]carbamate

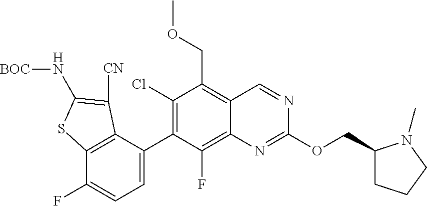 [SMILES: COCc1c(Cl)c(-c2ccc(F)c3sc(NC(=O)OC(C)(C)C)c(C#N)c23)c(F)c2nc(OC[C@@H]3CCCN3C)ncc12]

A solution of lithium bis(trimethylsilyl)amide in THE (1.5 M, 1.2 mL, 1.8 mmol) was added to a solution of N-methyl-L-prolinol (0.22 mL, 1.8 mmol) in THE (8 mL). The resulting mixture was stirred for 5 min. and treated with a solution of tert-butyl N-[4-[6-chloro-2-ethylsulfonyl-8-fluoro-5-(methoxymethyl)quinazolin-7-yl]-3-cyano-7-fluoro-benzothiophen-2-yl]carbamate (0.502 g, 0.82 mmol) in THE (5 mL). Stirring was continued at ambient temperature for 15 min. The reaction mixture was quenched with sat. aq. NH₄Cl and was diluted with EtOAc. The layers were separated and the aqueous layer was extracted with EtOAc. The organic layers were combined, dried over Na₂SO₄, filtered, and concentrated in vacuo. The residue was purified by flash silica gel chromatography (eluting with a gradient of 0 to 10% MeOH in DCM) to give the product (0.53 g, quantitative) as a yellow solid. ES/MS (m/z): 630.4 (M+H).

### Example 43

### 2-Amino-4-[6-chloro-8-fluoro-5-(methoxymethyl)-2-[[(2S)-1-methylpyrrolidin-2-yl]methoxy]quinazolin-7-yl]-7-fluoro-benzothiophene-3-carbonitrile, Isomer 1

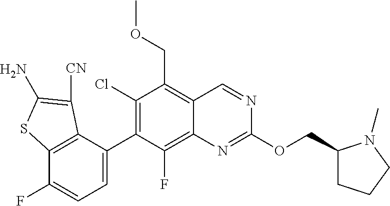 [SMILES: COCc1c(Cl)c(-c2ccc(F)c3sc(N)c(C#N)c23)c(F)c2nc(OC[C@@H]3CCCN3C)ncc12]

A mixture of tert-butyl N-[4-[6-chloro-8-fluoro-5-(methoxymethyl)-2-[[(2S)-1-methylpyrrolidin-2-yl]methoxy]quinazolin-7-yl]-3-cyano-7-fluoro-benzothiophen-2-yl]carbamate (0.200 g, 0.32 mmol) and 1,1,1,3,3,3-hexafluoro-2-propanol (5 mL) was heated at 120° C. for 1.5 h in a sealed vessel. The reaction mixture was cooled and concentrated under high vacuum. EtOAc was added and the mixture was concentrated (the process was repeated). The residue was purified by silica gel chromatography (eluting with a gradient of 0 to 10% MeOH in DCM) to give the mixture of atropisomers (105 mg). The mixture of atropisomers was separated by SFC using a CHIRALPAK® AS-H (21×250 cm), 80/20 CO₂/MeOH w/0.2% isopropylamine (Flow=80 mL/min, UV detection wavelength=225 nM, column temperature: 40° C.) to give the title compound as Isomer 1 (0.032 g, 31%, ee>99%) as a solid. ES/MS (m/z): 530.4 (M+H).

### Preparation 97

### (6-Chloro-2-ethylsulfanyl-8-fluoro-7-iodo-quinazolin-5-yl)methanol

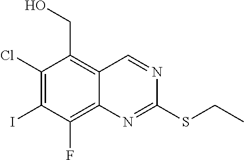 [SMILES: CCSc1ncc2c(CO)c(Cl)c(I)c(F)c2n1]

In a sealed reaction vessel, a solution of 6-chloro-2-ethylsulfanyl-8-fluoro-7-iodo-5-(methoxymethyl)quinazoline (0.293 g, 0.71 mmol) in DCM (20 mL) was cooled in an ice-water bath. A 1M solution of BBr₃ in DCM (1.4 mL, 1.4 mmol) was added. After stirring cold for 1.5 h, the reaction mixture was diluted with DCM and quenched carefully with sat. aq. NaHCO₃ until the aqueous layer was basic by pH. A yellow precipitate resulted and the mixture was stirred vigorously for 5 min. DCM (100 mL) was added, and the layers were separated. The aqueous layer was extracted with EtOAc and DCM. The organic layers were combined, dried over Na₂SO₄, filtered and concentrated in vacuo to obtain 337 mg of a ˜9:1 mixture of 5-(bromomethyl)-6-chloro-2-ethylsulfanyl-8-fluoro-7-iodo-quinazoline and (6-chloro-2-ethylsulfanyl-8-fluoro-7-iodo-quinazolin-5-yl)methanol. The 9:1 mixture was suspended in 1,4-dioxane (30 mL) and H₂O (30 mL). Cs₂CO₃ (2.0 g, 6.1 mmol) was added and the resulting mixture was heated at 50° C. overnight. The reaction was cooled to rt and diluted with EtOAc and sat. aq. NaCl. The layers were separated and the aqueous layer was extracted 2× with EtOAc. The organic layers were combined and were dried over Na₂SO₄. The solution was treated with activated charcoal, filtered, and concentrated in vacuo to afford the product (0.253 g, 86% purity) as a tan solid. ES/MS (m z): 399.2 (M+H).

### Preparation 98

### tert-Butyl-[(6-chloro-2-ethylsulfanyl-8-fluoro-7-iodo-quinazolin-5-yl)methoxy]-dimethyl-silane

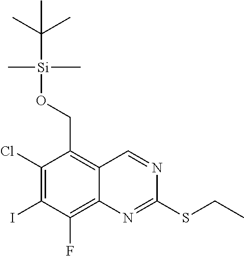 [SMILES: CCSc1ncc2c(CO[Si](C)(C)C(C)(C)C)c(Cl)c(I)c(F)c2n1]

A suspension of (6-chloro-2-ethylsulfanyl-8-fluoro-7-iodo-quinazolin-5-yl)methanol (0.373 g, 0.805 mmol, 86% purity) in DCM (20 mL) was cooled in an ice-water bath and was treated with 2,6-lutidine (0.175 mL, 1.50 mmol) and tert-butyldimethylsilyl trifluoromethanesulfonate (0.250 mL, 1.09 mmol). The cooling bath was removed and the mixture was allowed to stir at ambient temperature. After 1 h, additional 2,6-lutidine (0.175 mL, 1.50 mmol) and tert-butyldimethylsilyl trifluoromethanesulfonate (0.250 mL, 1.09 mmol) were added and the resulting mixture was stirred for 20 min. The reaction mixture was cooled in an ice-water bath and was quenched with sat. aq. NH₄Cl, diluted with DCM and sat. aq. NaCl solution. The layers were separated and the aqueous layer was extracted with DCM. The organic layers were combined, dried over Na₂SO₄, filtered and concentrated in vacuo. The residue was purified by silica gel chromatography (eluting with a gradient from 100% hexanes to 10% EtOAc in hexanes) to give the product (0.305 g, 70%) as a white solid. ES/MS (m/z): 513.4 (M+H).

### Preparation 99

### tert-Butyl N-[4-[5-[[tert-butyl(dimethyl)silyl]oxymethyl]-6-chloro-2-ethylsulfanyl-8-fluoro-quinazolin-7-yl]-3-cyano-7-fluoro-benzothiophen-2-yl]carbamate

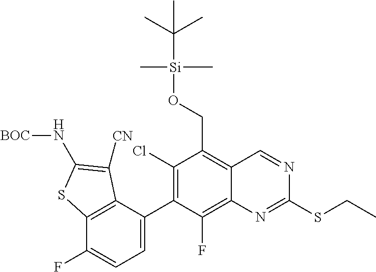 [SMILES: CCSc1ncc2c(CO[Si](C)(C)C(C)(C)C)c(Cl)c(-c3ccc(F)c4sc(NC(=O)OC(C)(C)C)c(C#N)c34)c(F)c2n1]

Prepared from tert-butyl-[(6-chloro-2-ethylsulfanyl-8-fluoro-7-iodo-quinazolin-5-yl)methoxy]-dimethyl-silane in a manner analogous to the method of Preparation 36 to afford the product (0.27 g, 64%). ES/MS (m z): 677.3 (M+H).

### Preparation 100

### tert-Butyl N-[4-[5-[[tert-butyl(dimethyl)silyl]oxymethyl]-6-chloro-2-ethylsulfonyl-8-fluoro-quinazolin-7-yl]-3-cyano-7-fluoro-benzothiophen-2-yl]carbamate

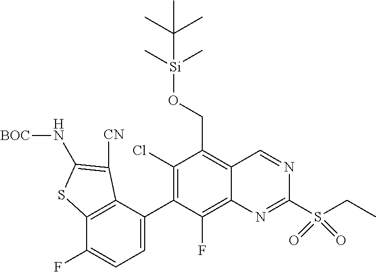 [SMILES: CCS(=O)(=O)c1ncc2c(CO[Si](C)(C)C(C)(C)C)c(Cl)c(-c3ccc(F)c4sc(NC(=O)OC(C)(C)C)c(C#N)c34)c(F)c2n1]

Prepared from tert-butyl N-[4-[5-[[tert-butyl(dimethyl)silyl]oxymethyl]-6-chloro-2-ethylsulfanyl-8-fluoro-quinazolin-7-yl]-3-cyano-7-fluoro-benzothiophen-2-yl]carbamate in a manner analogous to the method of Preparation of 95 to afford the product (0.28 g, quantitative) as a white solid. ES/MS (m z): 709.0 (M+H).

### Preparation 101

### tert-Butyl N-[4-[5-[[tert-butyl(dimethyl)silyl]oxymethyl]-6-chloro-8-fluoro-2-[[(2S)-1-methylpyrrolidin-2-yl]methoxy]quinazolin-7-yl]-3-cyano-7-fluoro-benzothiophen-2-yl]carbamate

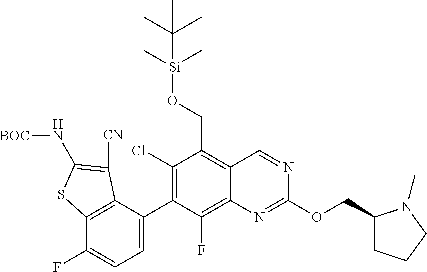 [SMILES: CN1CCC[C@H]1COc1ncc2c(CO[Si](C)(C)C(C)(C)C)c(Cl)c(-c3ccc(F)c4sc(NC(=O)OC(C)(C)C)c(C#N)c34)c(F)c2n1]

Prepared from tert-butyl N-[4-[5-[[tert-butyl(dimethyl)silyl]oxymethyl]-6-chloro-2-ethylsulfonyl-8-fluoro-quinazolin-7-yl]-3-cyano-7-fluoro-benzothiophen-2-yl]carbamate in a manner analogous to the method of Preparation of 96 to afford the product (0.26 g, 89%) as a yellow oil. ES/MS (m z): 730.0 (M+H).

### Example 44

### 2-Amino-4-[6-chloro-8-fluoro-5-(hydroxymethyl)-2-[[(2S)-1-methylpyrrolidin-2-yl]methoxy]quinazolin-7-yl]-7-fluoro-benzothiophene-3-carbonitrile, Isomer 1

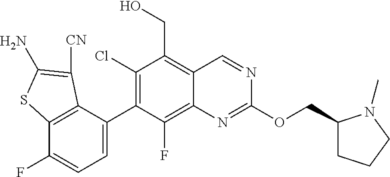 [SMILES: CN1CCC[C@H]1COc1ncc2c(CO)c(Cl)c(-c3ccc(F)c4sc(N)c(C#N)c34)c(F)c2n1]

A mixture of tert-butyl N-[4-[5-[[tert-butyl(dimethyl)silyl]oxymethyl]-6-chloro-8-fluoro-2-[[(2S)-1-methylpyrrolidin-2-yl]methoxy]quinazolin-7-yl]-3-cyano-7-fluoro-benzothiophen-2-yl]carbamate (0.250 g, 0.342 mmol) in TFA (5 mL) was treated with H₂O (0.5 mL). The resulting mixture was stirred at ambient temperature for 20 min. The solvent was removed in vacuo. The residue was treated with DCM and concentrated, repeated. The residue was dissolved in DCM and sat. aq. Na₂CO₃ was added to bring the pH ˜8. The layers were separated and the aqueous layer was extracted three more times with DCM. The organic layers were combined, dried over Na₂SO₄, filtered and concentrated in vacuo. The resulting white solid was purified by flash silica gel chromatography, eluting with a gradient of 2% to 10% (7 N ammoniated MeOH) in DCM, to give the mixture of atropisomers (0.134 g, 76%). The mixture of atropisomers (0.129 g) was separated by SFC (CHIRALPAK® IC, 21×250 mm; eluting with a mobile phase of 40% MeOH (with 0.5% DMEA) in 60% CO₂; column temperature: 40° C.; flow rate: 80 mL/minute; UV detection wavelength: 225 nm) to give the title compound (0.046 g, >97% ee) as the first eluting enantiomer (Isomer 1). ES/MS (m/z): 516.0 (M+H).

### Biological Assays

The following assays demonstrate that the exemplified compounds are inhibitors of KRas G12D and inhibit growth of certain tumors in vitro and/or in vivo.

### PANC-1 Cellular Active RAS GTPase ELISA (KRas G12D Mutation)

The purpose of this assay is to measure the ability of test compounds to inhibit constitutive RAS GTPase activity in human PANC-1 (RRID:CVCL_0480) pancreatic ductal adenocarcinoma cells (Supplier: ATCC #CRL-1469). The RAS GTPase ELISA kit (Active Motif Cat #52097) contains a 96-well glutathione-coated capture plate and kit-supplied Glutathione-S-Transferase (GST)-fused to Raf-Ras Binding Domain (RBD) protein. Activated pan-RAS (GTP-bound) in cell extracts specifically bind to the Raf-RBD. Bound RAS is detected with a primary Ras antibody that recognizes human K-Ras (and H-Ras). An HRP-conjugated anti-rat IgG secondary antibody recognizes the primary antibody, and a development substrate solution facilitates a chemiluminescent readout.

PANC-1 cells are plated at a concentration of 75,000 cells/well in 80 μL complete media (DMEM, high-glucose, L-glutamine, GIBCO; 10% heat-inactivated fetal bovine serum, GIBCO) and incubated overnight at 37° C./5% CO₂. Approximately 24 hours later, 20 μL of (1:3) serially-diluted (in complete media) test compound (1-50 μM top concentration) and 20 μL of serially-diluted (in complete media) controls (Maximum signal wells: 0.5% DMSO and Minimum signal wells: 10 μM reference positive control compound) are added to the cell plate and incubated for 2 hours at 37° C./5% CO₂. Complete Lysis/Binding Buffer is prepared containing Protease Inhibitor cocktail (PIC) and stored on ice. One hour before cell plate incubation is completed, GST-Raf-RBD is diluted in lysis/binding buffer, and 50 μL of mixed buffer per well is added to the supplied opaque white ELISA assay plate and is incubated for a minimum of 1 hour at 4° C., with gently rocking. After 2 hours, the cells are washed with 100 μL ice-cold Ca2+/Mg2+-free PBS and lysed with 100 μL of kit supplied lysis/binding buffer (AM11). After 30-50 minutes of vigorous plate shaking at ambient temperature, cell plate is centrifuged at 410×g (approx. 1500 rpm) for 10 minutes. Wash buffer diluted to 1× with ultrapure H₂O and 0.2 μm filtered is prepared at ambient temperature during the centrifugation step and then used to wash (3×100 μL) the GST-Raf-RBD coated assay plate. Next, 50 μL of cell lysate is added to the GST-Raf-RBD coated assay plate and incubated for 1 hour at ambient temperature with gentle shaking. During this incubation period, 1× Antibody Binding Buffer is prepared from thawed concentrate. The assay plate is washed 3×100 μL with 1× Wash Buffer, and then 50 μL of Primary RAS Antibody (kit supplied #101678), diluted 1:500 in 1× Antibody Binding buffer, is added. After a one hour of ambient incubation with gentle shaking, the assay plate is washed 3×100 μL with 1× Wash Buffer. Subsequently, 50 μL of Anti-rat HRP-conjugated IgG secondary antibody (0.25 μg/μL) (diluted 1:5000 in 1× Antibody Binding buffer) is added to each well of the assay plate, and incubated an additional hour at ambient temperature with gentle shaking. Finally, the assay plate is washed 4×100 μL with 1× Wash buffer, followed by addition of 50 μL of mixed ambient temperature chemiluminescent working solution (combination of Reaction buffer with a chemiluminescence substrate). Data from each well's luminescent emission is recorded with a 2104 EnVision™ Plate Reader (Perkin Elmer) using a luminescence program optimized for the assay plate dimensions.

The signal is converted to percent inhibition using the following equation: % Inhibition=100−[(Test Compound Signal−Median Minimum Signal)/(Median Maximum Signal−Median Minimum Signal)×100]. The Maximum signal is a control well without inhibitor (DMSO). The Minimum signal is a control well containing a reference inhibitor sufficient to fully inhibit activity. The IC₅₀ is determined by fitting the percent inhibition at each inhibitor concentration to the four parameter nonlinear logistic equation using Genedata Screener®, v17: y=(A+((B−A)/(1+((x/C)\hat{ }D)))) where, y=% inhibition, A=minimum asymptote, B=maximum asymptote, C=relative IC₅₀ or the inhibitor concentration producing 50% inhibition within the fitted range of both asymptotes, and D=Hill Slope.

Compounds of Formulae I, II, III, or IV as described herein and shown in Table 1 were evaluated in this assay substantially as described. The compounds exhibited an ability to inhibit constitutive RAS GTPase activity indicating inhibition of KRas G12D mutant enzyme. About three-quarters of the example compounds of Table 1 herein exhibited a relative IC₅₀ of <500 nM in this assay. Of these, the preferred example compounds of Table 2 exhibited a relative IC₅₀ of <100 nM in this assay. This data shows that compounds of Formulae I, II, III, or IV as described herein are capable of inhibiting KRAS-GTP activity in this human pancreatic cancer cell culture demonstrating the ability to inhibit KRas G12D mutants.

### MKN-45 Cellular Active RAS GTPase ELISA (KRas Wild-Type)

The purpose of this assay is to measure the ability of test compounds to inhibit constitutive RAS GTPase activity in human MKN-45 gastric adenocarcinoma cell (Supplier: JCRB, SupplierID: JCRB 0254, Lot:05222009). The RAS GTPase ELISA kit (Active Motif Cat #52097) contains a 96-well glutathione-coated capture plate and kit-supplied Glutathione-S-Transferase (GST)-fused to Raf-Ras Binding Domain (RBD) protein. Activated pan-RAS (GTP-bound) in cell extracts specifically bind to the Raf-RBD. Bound RAS is detected with a primary Ras antibody that recognizes human K-Ras (and H-Ras). An HRP-conjugated anti-rat IgG secondary antibody recognizes the primary antibody, and a development substrate solution facilitates a chemiluminescent readout.

MKN-45 cells are plated at a concentration of 75,000 cells/well in 80 μL complete media (DMEM, high-glucose, L-glutamine, GIBCO; 10% heat-inactivated fetal bovine serum, GIBCO) and incubated overnight at 37° C./5% CO₂. Approximately 24 hours later, 20 μL of (1:3) serially-diluted (in complete media) test compound (1-10 μM top concentration) and 20 μL of serially-diluted (in complete media) controls (Maximum signal wells: 0.1% DMSO and Minimum signal wells: 10 μM reference positive control compound) are added to the cell plate and incubated for 2 hours at 37° C./5% CO₂. Complete Lysis/Binding Buffer is prepared containing Protease Inhibitor cocktail (PIC) and stored on ice. One hour before cell plate incubation is completed, GST-Raf-RBD is diluted in lysis/binding buffer, and 50 μL of mixed buffer per well is added to the supplied opaque white ELISA assay plate and is incubated for a minimum of 1 hour at 4° C., with gently rocking. After 2 hours, the cells are washed with 100 μL ice-cold Ca2+/Mg2+-free PBS and lysed with 100 μL of kit supplied lysis/binding buffer (AM11). After 30-50 minutes of vigorous plate shaking at ambient temperature, cell plate is centrifuged at 410×g (approx. 1500 rpm) for 10 minutes. Wash buffer diluted to 1× with ultrapure H₂O during the centrifugation step and then used to wash (3×100 μL) the GST-Raf-RBD coated assay plate. Next, 50 μL of cell lysate is added to the GST-Raf-RBD coated assay plate and incubated for 1 hour at ambient temperature with gentle shaking. During this incubation period, 1× Antibody Binding Buffer is prepared from thawed concentrate. The assay plate is washed 3×100 μL with 1× Wash Buffer, and then 50 μL of Primary RAS Antibody (kit supplied #101678), diluted 1:500 in 1× Antibody Binding buffer, is added. After a one hour of ambient incubation with gentle shaking, the assay plate is washed 3×100 μL with 1× Wash Buffer. Subsequently, 50 μL of Anti-rat HRP-conjugated IgG secondary antibody (0.25 μg/μL) (diluted 1:5000 in 1× Antibody Binding buffer) is added to each well of the assay plate and incubated an additional hour at ambient temperature with gentle shaking. Finally, the assay plate is washed 4×100 μL with 1× Wash buffer, followed by addition of 50 μL of mixed ambient temperature chemiluminescent working solution (combination of Reaction buffer with a chemiluminescence substrate). Data from each well's luminescent emission is recorded with a 2104 EnVision™ Plate Reader (Perkin Elmer) using a luminescence program optimized for the assay plate dimensions.

The signal is converted to percent inhibition using the following equation: % Inhibition=100−[(Test Compound Signal−Median Minimum Signal)/(Median Maximum Signal−Median Minimum Signal)×100]. The Maximum signal is a control well without inhibitor (DMSO). The Minimum signal is a control well containing a reference inhibitor sufficient to fully inhibit activity. The IC₅₀ is determined by fitting the percent inhibition at each inhibitor concentration to the four parameter nonlinear logistic equation using Genedata Screener®, v17: y=(A+((B−A)/(1+((x/C)\hat{ }D)))) where, y=% inhibition, A=minimum asymptote, B=maximum asymptote, C=relative IC₅₀ or the inhibitor concentration producing 50% inhibition within the fitted range of both asymptotes, and D=Hill Slope.

A subset of compounds of Formulae I, II, III, or IV as described herein (Examples 3, 4, 7, 8, 15, 17, 21, 26, and 33) were evaluated in this assay substantially as described. All the compounds tested in this assay were also tested and showed inhibitory activity in the KRas G12D mutant assay above. Most of the compounds tested in this assay exhibited some ability to inhibit constitutive RAS GTPase activity (i.e., KRas wild-type inhibition). The compounds of examples 3, 4, 7, and 33 showed a significant (i.e., greater than 7-fold) selective inhibition preference for KRas G12D mutant over KRas wild-type demonstrating the potential for compounds of Formulae I, II, III, or IV as described herein to be both potent and selective inhibitors of KRas G12D mutants.

1. A compound of the formula: 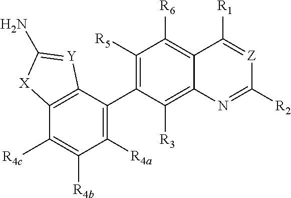
wherein:
X is —O— or —S—;
Y is —C(CN)— or —N—;
Z is —C(H)— or —N—;
R₁ is H, azetidine, pyrrolidine, piperidine, or N-linked piperazine, wherein the azetidine, pyrrolidine, piperidine, or N-linked piperazine are optionally substituted with C₁₋₄ alkyl or C₂₋₄ heteroalkyl, wherein the C₁₋₄ alkyl, C₂₋₄ heteroalkyl are optionally substituted by halogen or oxo, wherein the azetidine, pyrrolidine, piperidine, or N-linked piperazine are optionally bridged by the C₁₋₄ alkyl or C₂₋₄ heteroalkyl, and wherein the azetidine, pyrrolidine, piperidine, or N-linked piperazine are optionally fused with the C₁₋₄ alkyl or C₂₋₄ heteroalkyl to form a bicyclic ring;
R₂ is H, —O—CH₂—R₇, or —O—CH(CH₃)—R₇, wherein R₇ is azetidine, pyrrolidine, or tetrahydrofuran, wherein the azetidine, pyrrolidine, or tetrahydrofuran are optionally substituted with one or more halogen, hydroxyl, C₁₋₄ alkyl, or C₁₋₄ alkenyl, wherein the C₁₋₄ alkyl is optionally substituted with one or more halogen or hydroxyl, wherein the azetidine, pyrrolidine, or tetrahydrofuran are optionally fused with the C₁₋₄ alkyl to form a bicyclic ring, and wherein if R₂ is H then R₁ is not H;
R₃ and R₅ are each independently H, halogen, —C₀₋₃ alkyl-cyclopropyl, —C₁₋₆ alkyl optionally substituted 1-3 times with R₈, or —O—C₁₋₆ alkyl optionally substituted 1-3 times with R₈;
R₄ₐ, R_{4b}, and R_{4c} are each independently H, halogen, or —C₁₋₆ alkyl optionally substituted 1-3 times with R₈;
R₆ is H, —CH₂OH, —CH₂—O—CH₃;
R₈ is independently at each occurrence halogen, oxo, hydroxy, —C₁₋₄ alkyl, or —O—C₁₋₄ alkyl;
or a pharmaceutically acceptable salt thereof.

2. The compound according to claim 1, wherein X is —S—, or a pharmaceutically acceptable salt thereof.

3. The compound according to claim 1, wherein Y is —C(CN)—, or a pharmaceutically acceptable salt thereof.

4. The compound according to claim 1, wherein Z is —N—, or a pharmaceutically acceptable salt thereof.

5. The compound according to claim 1, wherein R₁ is H, or a pharmaceutically acceptable salt thereof.

6. The compound according to claim 1, wherein R₁ is azetidine, pyrrolidine, piperidine, or N-linked piperazine, or a pharmaceutically acceptable salt thereof.

7. The compound according to claim 6, wherein R₁ is N-linked piperazine, or a pharmaceutically acceptable salt thereof.

8. The compound according to claim 6, wherein R₁ is 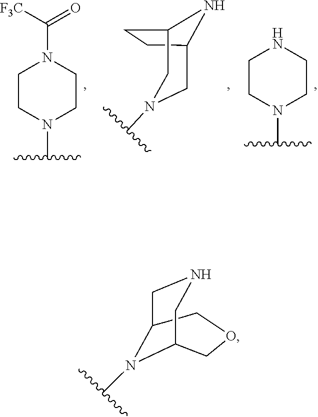 [SMILES: *N1CCN(C(=O)C(F)(F)F)CC1 ; *N1CC2CCC(C1)N2 ; *N1CCNCC1 ; *N1C2CCOCC1CNC2]
or a pharmaceutically acceptable salt thereof.

9. The compound according to claim 6, wherein R₁ is 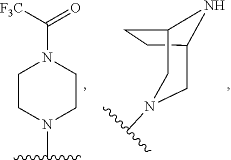 [SMILES: *N1CCN(C(=O)C(F)(F)F)CC1 ; *N1CC2CCC(C1)N2]
or a pharmaceutically acceptable salt thereof.

10. The compound according to claim 1, wherein R₂ is —O—CH₂—R₇ or —O—CH(CH₃)—R₇, or a pharmaceutically acceptable salt thereof.

11. The compound according to claim 8, wherein R₂ is —O—CH₂—R₇, or a pharmaceutically acceptable salt thereof.

12. The compound according to claim 10, wherein R₇ is pyrrolidine, or a pharmaceutically acceptable salt thereof.

13. The compound according to claim 1, wherein R₂ is 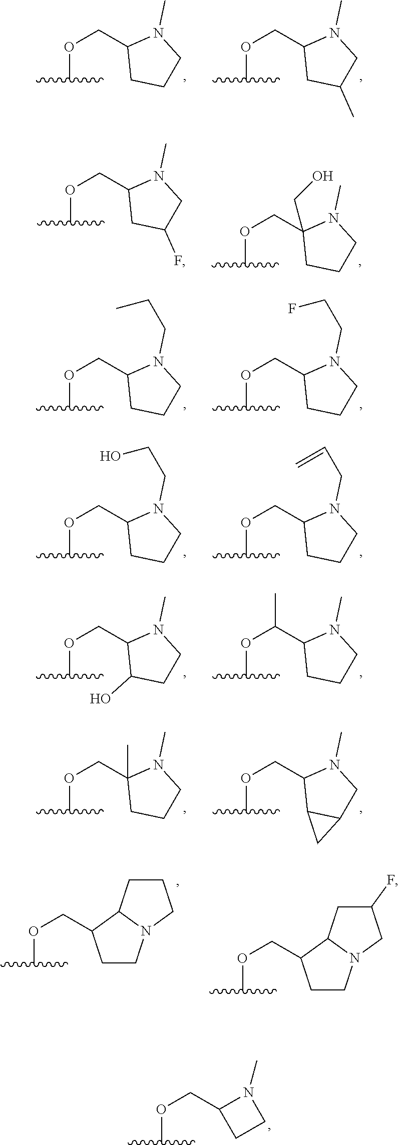
or a pharmaceutically acceptable salt thereof.

14. The compound according to claim 1, wherein R₂ is 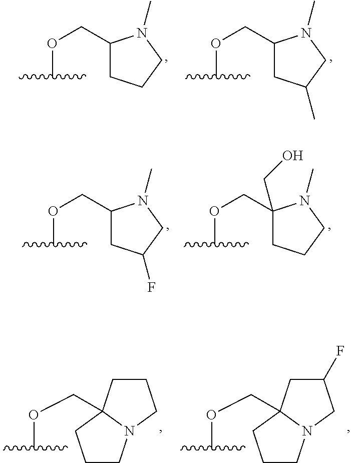
or a pharmaceutically acceptable salt thereof.

15. The compound according to claim 1, wherein R₃ and R₅ are each independently halogen, —C₀₋₃ alkyl-cyclopropyl, —C₁₋₆ alkyl optionally substituted 1-3 times with R₈, or —O—C₁₋₆ alkyl optionally substituted 1-3 times with R₈.

16. The compound according to claim 1, wherein R₃ is F, or a pharmaceutically acceptable salt thereof.

17. The compound according to claim 1, wherein R_{4c} is F or —CH₃, or a pharmaceutically acceptable salt thereof.

18. The compound according to claim 1, wherein R₅ is Cl, or a pharmaceutically acceptable salt thereof.

19. The compound according to claim 1, wherein X is S, Y is —C(CN)—, R₃ is F, R₄ₐ is H, R_{4b} is H, R_{4c} is F, and R₅ is Cl, or a pharmaceutically acceptable salt thereof.

20. The compound according to claim 1 selected from: 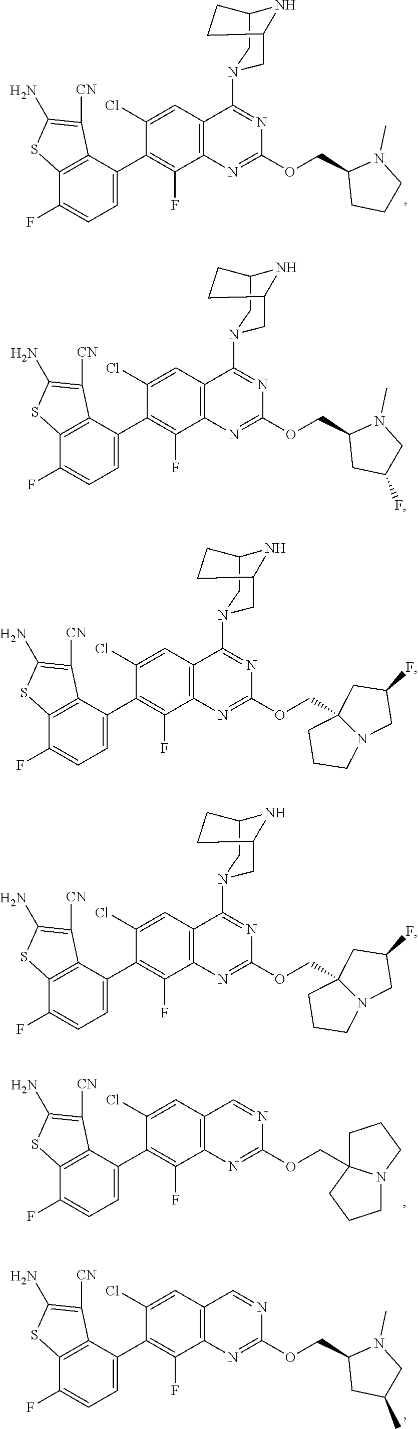 [SMILES: CN1CCC[C@H]1COc1nc(N2CC3CCC(C2)N3)c2cc(Cl)c(-c3ccc(F)c4sc(N)c(C#N)c34)c(F)c2n1 ; CN1C[C@H](F)C[C@H]1COc1nc(N2CC3CCC(C2)N3)c2cc(Cl)c(-c3ccc(F)c4sc(N)c(C#N)c34)c(F)c2n1 ; N#Cc1c(N)sc2c(F)ccc(-c3c(Cl)cc4c(N5CC6CCC(C5)N6)nc(OC[C@@]56CCCN5C[C@H](F)C6)nc4c3F)c12 ; N#Cc1c(N)sc2c(F)ccc(-c3c(Cl)cc4c(N5CC6CCC(C5)N6)nc(OC[C@@]56CCCN5C[C@H](F)C6)nc4c3F)c12 ; N#Cc1c(N)sc2c(F)ccc(-c3c(Cl)cc4cnc(OCC56CCCN5CCC6)nc4c3F)c12 ; C[C@H]1C[C@@H](COc2ncc3cc(Cl)c(-c4ccc(F)c5sc(N)c(C#N)c45)c(F)c3n2)N(C)C1] 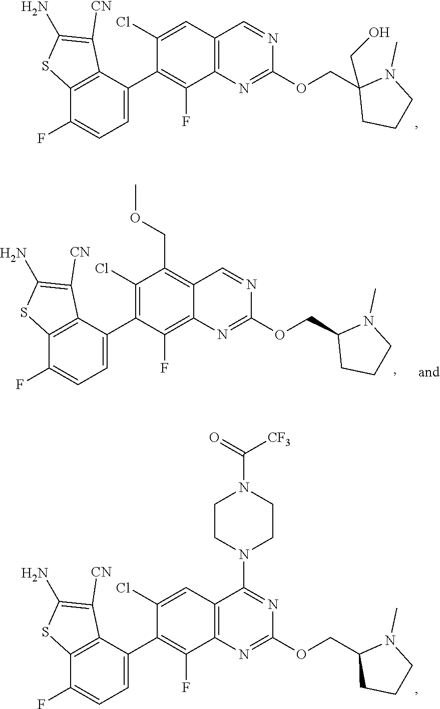 [SMILES: CN1CCCC1(CO)COc1ncc2cc(Cl)c(-c3ccc(F)c4sc(N)c(C#N)c34)c(F)c2n1 ; COCc1c(Cl)c(-c2ccc(F)c3sc(N)c(C#N)c23)c(F)c2nc(OC[C@@H]3CCCN3C)ncc12 ; CN1CCC[C@H]1COc1nc(N2CCN(C(=O)C(F)(F)F)CC2)c2cc(Cl)c(-c3ccc(F)c4sc(N)c(C#N)c34)c(F)c2n1]
or a pharmaceutically acceptable salt thereof.

21. The compound according to claim 20, which is:

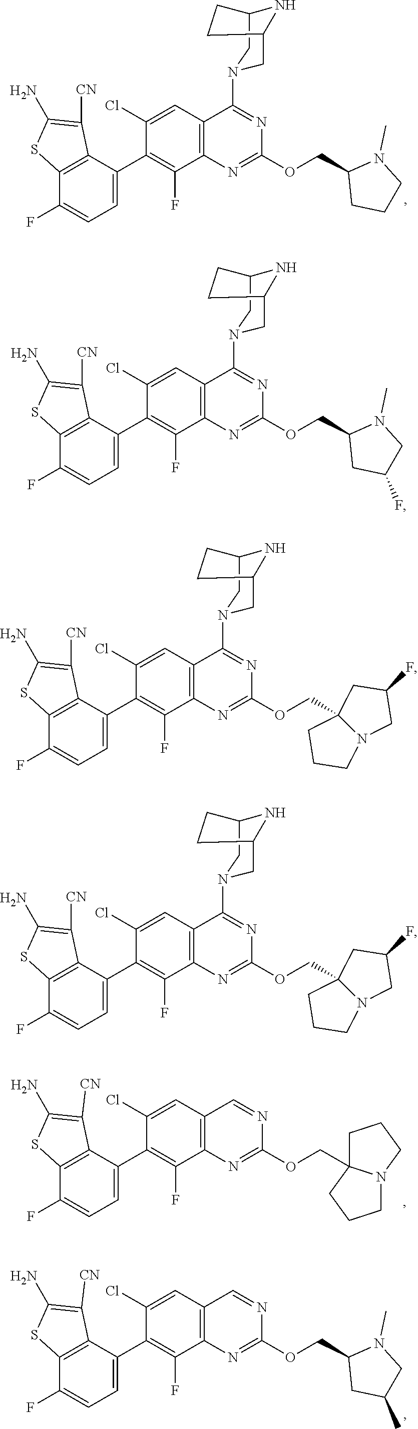 [SMILES: CN1CCC[C@H]1COc1nc(N2CC3CCC(C2)N3)c2cc(Cl)c(-c3ccc(F)c4sc(N)c(C#N)c34)c(F)c2n1 ; CN1C[C@H](F)C[C@H]1COc1nc(N2CC3CCC(C2)N3)c2cc(Cl)c(-c3ccc(F)c4sc(N)c(C#N)c34)c(F)c2n1 ; N#Cc1c(N)sc2c(F)ccc(-c3c(Cl)cc4c(N5CC6CCC(C5)N6)nc(OC[C@@]56CCCN5C[C@H](F)C6)nc4c3F)c12 ; N#Cc1c(N)sc2c(F)ccc(-c3c(Cl)cc4c(N5CC6CCC(C5)N6)nc(OC[C@@]56CCCN5C[C@H](F)C6)nc4c3F)c12 ; N#Cc1c(N)sc2c(F)ccc(-c3c(Cl)cc4cnc(OCC56CCCN5CCC6)nc4c3F)c12 ; C[C@H]1C[C@@H](COc2ncc3cc(Cl)c(-c4ccc(F)c5sc(N)c(C#N)c45)c(F)c3n2)N(C)C1]

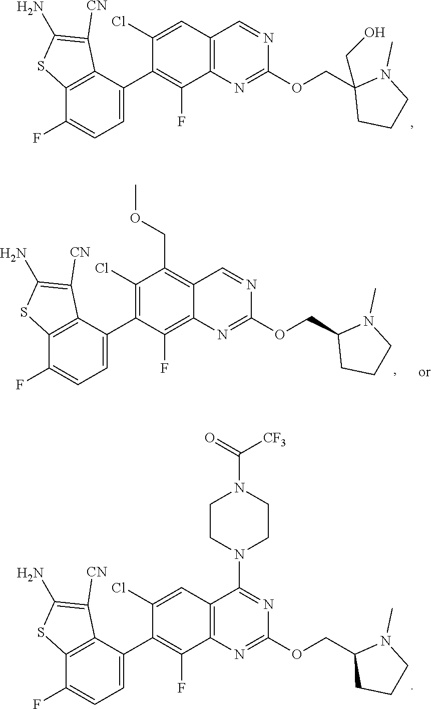 [SMILES: CN1CCCC1(CO)COc1ncc2cc(Cl)c(-c3ccc(F)c4sc(N)c(C#N)c34)c(F)c2n1 ; COCc1c(Cl)c(-c2ccc(F)c3sc(N)c(C#N)c23)c(F)c2nc(OC[C@@H]3CCCN3C)ncc12 ; CN1CCC[C@H]1COc1nc(N2CCN(C(=O)C(F)(F)F)CC2)c2cc(Cl)c(-c3ccc(F)c4sc(N)c(C#N)c34)c(F)c2n1]

22. A pharmaceutical composition comprising a compound according to claim 1, or a pharmaceutically acceptable salt thereof, and a pharmaceutically acceptable carrier, diluent or excipient.

23. A method of treating a patient for cancer, comprising administering to a patient in need thereof, an effective amount of a pharmaceutical composition according to claim 22, wherein the cancer is selected from lung cancer, pancreatic cancer, cervical cancer, esophageal cancer, endometrial cancer, ovarian cancer, cholangiocarcinoma, and colorectal cancer.

24. A method of treating a patient for cancer, comprising administering to a patient in need thereof, an effective amount of a compound according to claim 1, or a pharmaceutically acceptable salt thereof, wherein the cancer is selected from lung cancer, pancreatic cancer, cervical cancer, esophageal cancer, endometrial cancer, ovarian cancer, cholangiocarcinoma, and colorectal cancer.

25. The method according to claim 24, wherein the cancer is non-small cell lung cancer, and wherein one or more cells express KRas G12D mutant protein.

26. The method according to claim 24, wherein the cancer is colorectal cancer, and wherein one or more cells express KRas G12D mutant protein.

27. The method according to claim 24, wherein the cancer is pancreatic cancer, and wherein one or more cells express KRas G12D mutant protein.

28. The method according to claim 24, wherein the patient has a cancer that was determined to have one or more cells expressing the KRas G12D mutant protein prior to administration of the compound or a pharmaceutically acceptable salt thereof.

29. A method of treating a patient with a cancer that has a KRas G12D mutation comprising administering to a patient in need thereof an effective amount of a compound according to claim 1, or a pharmaceutically acceptable salt thereof.

30. The method according to claim 24, wherein the patient is also administered an effective amount of one or more of a PD-1 inhibitor, a PD-L1 inhibitor, a CD4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof.

31. (canceled)

32. (canceled)

33. (canceled)

34. (canceled)
